(19) United States
(12) Patent Application Publication
(10) Pub. No.: US 2026/0167678 A1
(43) Pub. Date: Jun. 18, 2026

(54) TRUNCATED CHIMERIC INSECTICIDAL PROTEINS

(71) Applicant: Empresa Brasileira de Pesquisa Agropecuária - Embrapa, Brasília (BR)

(72) Inventors: Newton PORTILHO CARNEIRO, Brasília (BR); Andréa ALMEIDA CARNEIRO, Brasília (BR); Fernando HERCOS VALICENTE, Brasília (BR); Simone MARTINS MENDES, Brasília (BR); Meire DE CASSIA ALVES, Brasília (BR); Beatriz DE ALMEIDA BARROS, Brasília (BR); Sidney NETTO PARENTONI, Brasília (BR); Ubiraci GOMES DE PAULA LANA, Brasília (BR)

(73) Assignee: Empresa Brasileira de Pesquisa Agropecuária - Embrapa, Brasília (BR)

(21) Appl. No.: 19/126,690

(22) PCT Filed: Oct. 19, 2023
(86) PCT No.: PCT/BR2023/050357

§ 371 Date: May 2, 2025

(30) Foreign Application Priority Data
Nov. 4, 2022 (BR) 1020220223963

(51) Int. Cl. C07K 14/325; A01N 63/50; A01P 7/04; C12N 15/82

(52) U.S. Cl. CPC C07K 14/325; A01N 63/50; A01P 7/04; C12N 15/8286

(57) ABSTRACT

The present invention refers to nucleotide sequences encoding new truncated chimeric insecticidal proteins that exhibit lepidopteran inhibitory activity. Particular embodiments provide compositions and plants, plant parts, transformed seeds containing the recombinant nucleic acid molecules, which encode one or more of the chimeric insecticidal proteins.

### FIELD OF THE INVENTION

The invention refers, in general, to the field of insect-inhibiting proteins useful in agriculture. The present patent application teaches a new class of chimeric insecticidal proteins that exhibit inhibitory activity against insect pests relevant to the agriculture of cultivated plants and seeds. In particular, the present patent application teaches a class of proteins with insecticidal activity against the Lepidoptera order of insect pests. Plants, plant parts, and seeds that contain a recombinant nucleic acid molecule encoding one or more insecticidal proteins are also taught.

### BACKGROUND OF THE INVENTION

There is a growing need for improving the productivity of crops of agriculturally significant plants, including, among others, cotton, soybeans, corn, sugarcane, rice, and wheat. In addition to the increasing demand for agricultural products for food, clothing, and energy supply for a growing human population, climate-related effects and increasing population pressure to use land for purposes other than agricultural practices tend to reduce the amount of land available for agriculture. These effects generate greater demand for productivity and efficiency of agricultural crops. In view of the growing demand, agricultural pest management and control techniques are vital tools to increase the production per unit of land available for agriculture.

Insects, particularly insects within the order Lepidoptera, are a major cause of damage to agricultural crops, reducing the yield of infested crops. Lepidopteran pest species that negatively impact agriculture include, for example, Agrotis ipsilon, Agrotis subterranea, Agrotis orthogonia, Alabama argillacea, Anticarsia gemmatalis, Amyelois transitella, Archips argyrospila, Archips rosana, Chilo suppressalis, Chrysodeixis includens, Cnaphalocrocis medinalis, Crambus caliginosellus, Crambus teterrellus, Cydia pomonella, Diatraea saccharalis, Diatraea grandiosella, Earias vitela, Earias insulana, Earias vittella, Elasmopalpus lignosellus, Endopiza viteana, Grapholita molesta, Helicoverpa armigera, Helicoverpa gelotopeon, Helicoverpa zea, Heliothis virescens, Herpetogramma licarsisalis, Homoeosoma electellum, Hypena scabra, Lobesia botrana, Lymantria dispar, Mamestra configurata, Ostrinia nubilalis, Pectinophora gossypiella, Pieris brassicae, Pieris rapae, Phyllocnistis citrella, Plutella xylostella, Pseudaletia unipuncta, Rachiplusia nu, Sesamia inferens, Spodoptera cosmioides, Spodoptera exigua, Spodoptera eridania, Spodoptera frugiperda, Spodoptera litura, Suleima helianthana, Trichoplusia ni, and Tuta absoluta.

Synthetic chemical insecticides have historically been used in the control of insect pests in agriculture. However, concerns about the environment and human health, as well as emerging resistance problems, have stimulated research and development of biological pesticides. This research effort has resulted in the progressive observation and use of several entomopathogenic microbial species, especially bacteria of the genus Bacillus.

The biological control paradigm was altered when the potential of entomopathogenic bacteria, especially bacteria belonging to the genus Bacillus, was disclosed. The identification of the entomopathogenic potential of various strains of Bacillus bacteria, especially Bacillus thuringiensis (Bt), represented a significant advancement in insect pest control techniques. A large family of proteins with high toxicity against specific insects has been identified in several strains of the bacterium Bt. Among others, delta-endotoxins that are located within parasporal crystalline inclusion bodies at the establishment of sporulation and during the stationary growth phase (e.g., Cry proteins) as well as secreted insecticidal proteins have been identified. The insecticidal proteins of Bt exert their effects on the surface of the epithelium of the midgut of insects after ingestion by disrupting the cell membrane, leading to cell rupture and death.

The secreted and crystalline insecticidal proteins of Bt are highly specific to their hosts and have been globally recognized and accepted as important alternatives to chemical insecticides. For example, insecticidal proteins are employed in various agricultural crops to protect crop plants against insect infestations, decrease the need for chemical pesticide application, and increase productivity. Bt insecticidal proteins are used to control agriculturally relevant pests of crop plants by applying microbial agricultural compositions to the plants and using genetic transformation techniques to obtain transgenic plants and seeds that express the insecticidal protein.

The global use of insect-resistant transgenic plant crops and the limited number of insecticidal proteins used in these crops have created a selection pressure for the insect resistance alleles to the insecticidal proteins currently used.

The development of resistance to insecticidal proteins in target pests generates a continuous demand for the identification and development of new proteins that control these resistant insect populations. New insecticidal proteins with improved efficacy and an alternative spectrum of activity against insect species are important in the control of resistant insects. In addition, the use of two or more insecticidal proteins in the same transgenic plant with different modes of action for the same insect pest reduces the probability of resistance in any target insect species.

Consequently, there is a constant demand for the identification of new proteins with improved insecticidal properties, such as efficacy, activity against more than one species of insect pests, and different modes of action compared to the toxins currently used in agricultural practices.

To satisfy this demand, the present invention describes new chimeric insecticidal Cry proteins that exhibit activity against important species of insect pests.

A Cry protein nomenclature system by amino acid similarity was originally proposed by Crickmore, N., et al., 1998. Revision of the nomenclature for the Bacillus thuringiensis pesticidal crystal proteins. Microbiol. Mol. Biol. Rev. 62, 807-813) and updated by Crickmore, N., et al., 2021. A structure-based nomenclature for Bacillus thuringiensis and other bacteria-derived pesticidal proteins. Journal of Invertebrate Pathology Vol. 186, (107438).

In the most recent classification, the Cry class of proteins represents proteins originally isolated from B. thuringiensis crystals whose precursor form consists of two similarly sized halves: an amino-terminal active portion subdivided into three domains based on the alignment of conserved or substantially conserved sequences, and a carboxy-terminal portion known to stabilize crystal formation and exhibit no insecticidal activity. Domain I of the active amino-terminal portion comprises about the first third of the active toxin segment and has been shown to be essential for channel formation. Domains II and III of the active amino-terminal portion have both been implicated in receptor binding and insect species specificity, depending on the insect and the insecticidal protein to be examined.

The probability of arbitrarily creating a chimeric protein with improved properties from the variety of domain structures of the numerous native insecticidal proteins known in the art is remote. This is a result of the complex and unpredictable nature of the protein structure, oligomerization, and activation (including correct proteolytic processing of the chimeric precursor, if expressed in such a way) necessary to release a segment of insecticidal protein. The creation of functional chimeric insecticidal toxins with enhanced insecticidal activity compared to the parental proteins from which the chimeras are derived involves careful selection of the subunits, domains, and specific targets within each parental protein.

It is known in the art that the reassembly of domains I, II, and III of any two or more toxins that are different from each other often results in the construction of proteins that exhibit crystal formation with a complete absence of any detectable insecticidal activity directed at a preferred target insect pest species. Only by trial and error are effective insecticidal chimeras designed, and even then, the subject matter technician cannot guarantee that he will obtain a chimera that exhibits insecticidal activity that is equivalent or enhanced compared to a single parent toxin protein from which the constituent protoxin or chimera toxin domains may have been derived. For example, literature reports numerous examples of constructing or assembling chimeric proteins from two or more crystal protein precursors. See, e.g., Jacqueline S. Knight, et al. “A Strategy for Shuffling Numerous Bacillus thuringiensis Crystal Protein Domains”. J. Economic Entomology, 97 (6) (2004): pages 1805 to 1813; U.S. Pat. Nos. 6,204,246; 6,017,534; and 10,233,217. In each of these examples, many of the resulting chimeras did not exhibit insecticidal or crystal-forming properties that were equivalent to or improved upon the precursor proteins from which the chimera components were derived.

### SUMMARY OF THE INVENTION

The present invention provides novel truncated chimeric Cry proteins, formed from the recombination of domains I, II, and III of the active amino-terminal portion of Bt parental Cry proteins Cry1Ab, Cry1B, Cry1C, Cry1Da, Cry1E, Cry1Fb, and Cry2Aa in the absence of a carboxy-terminal protoxin domain. The truncated chimeric proteins of the present invention have inhibitory and toxic activity against insect pests of crops of the order Lepidoptera. Also objects of the present invention are nucleic acid molecules encoding the aforementioned truncated chimeric proteins, expression cassettes, plant cells, plants, plant parts, and seeds containing a recombinant nucleic acid molecule encoding one or more of the truncated chimeric proteins.

Each of the truncated chimeric insecticidal proteins can be used alone or in combination with other insecticidal proteins and insect inhibitors in formulations and for expression in plants. In this way, alternatives to insecticidal proteins and chemical insecticides used in agricultural systems are provided.

In certain embodiments described in this document, a truncated chimeric insecticidal protein comprises (i) a Domain I of a Cry protein from any of SEQ ID NO: 98, SEQ ID NO: 102, SEQ ID NO: 106, SEQ ID NO: 111, SEQ ID NO: 115, SEQ ID NO: 119 or SEQ ID NO: 123; (ii) a Domain II of a Cry protein from any of SEQ ID NO: 99, SEQ ID NO: 103, SEQ ID NO: 107, SEQ ID NO: 112, SEQ ID NO: 116, SEQ ID NO: 120 or SEQ ID NO: 124; and (iii) a Domain III of Cry protein from any of SEQ ID NO: 100, SEQ ID NO: 104, SEQ ID NO: 108, SEQ ID NO: 109, SEQ ID NO: 113, SEQ ID NO: 117, SEQ ID NO: 121 or SEQ ID NO: 125 and lacks a carboxy-terminal protoxin domain.

In preferential embodiment, a truncated chimeric insecticidal protein comprises a sequence of amino acids as established in any of the SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48, and lacks a carboxy-terminal protoxin domain.

Chimeric insecticidal proteins have inhibitory activity against an insect species of the order Lepidoptera such as, but not limited to, Agrotis ipsilon, Agrotis subterranea, Agrotis orthogonia, Alabama argillacea, Anticarsia gemmatalis, Amyelois transitella, Archips argyrospila, Archips rosana, Chilo suppressalis, Chrysodeixis includens, Cnaphalocrocis medinalis, Crambus caliginosellus, Crambus teterrellus, Cydia pomonella, Diatraea saccharalis, Diatraea grandiosella, Earias vitella, Earias insulana, Earias vittella, Elasmopalpus lignosellus, Endopiza viteana, Grapholita molesta, Helicoverpa armigera, Helicoverpa gelotopeon, Helicoverpa zea, Heliothis virescens, Herpetogramma licarsisalis, Homoeosoma electellum, Hypena scabra, Lobesia botrana, Lymantria dispar, Mamestra configurata, Ostrinia nubilalis, Pectinophora gossypiella, Pieris brassicae, Pieris rapae, Phyllocnistis citrella, Plutella xylostella, Pseudaletia unipuncta, Rachiplusia nu, Sesamia inferens, Spodoptera cosmioides, Spodoptera exigua, Spodoptera eridania, Spodoptera frugiperda, Spodoptera litura, Suleima helianthana, Trichoplusia ni, and Tuta absoluta.

In another embodiment, a polynucleotide is provided that encodes a truncated chimeric insecticidal protein, where the truncated chimeric insecticidal protein comprises (i) a Cry protein Domain I of any of the SEQ ID NO: 98, SEQ ID NO: 102, SEQ ID NO: 106, SEQ ID NO: 111, SEQ ID NO: 115, SEQ ID NO: 119 or SEQ ID NO: 123; (ii) a Domain II of a Cry protein from any of SEQ ID NO: 99, SEQ ID NO: 103, SEQ ID NO: 107, SEQ ID NO: 112, SEQ ID NO: 116, SEQ ID NO: 120 or SEQ ID NO: 124; and (iii) a Domain III of Cry protein from any of SEQ ID NO: 100, SEQ ID NO: 104, SEQ ID NO: 108, SEQ ID NO: 109, SEQ ID NO: 113, SEQ ID NO: 117, SEQ ID NO: 121 or SEQ ID NO: 125, in which the protein lacks a carboxy-terminal protoxin domain. In a preferential embodiment, the chimeric protein insecticide comprises the amino acid sequence of any of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48, where the amino acid sequence lacks a carboxy-terminal protoxin domain. A polynucleotide is also provided that encodes a truncated chimeric insecticidal protein, in which the polynucleotide comprises a sequence of nucleotides that optionally: hybridizes under stringent conditions with the reverse complement of the polynucleotide sequence of any of SEQ ID NOs: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96, where the polynucleotide lacks a region coding for a carboxy-terminal protoxin domain; or encodes the truncated chimeric insecticidal protein comprising (i) a Cry Protein Domain I of any of SEQ ID NO: 98, SEQ ID NO: 102, SEQ ID NO: 106, SEQ ID NO: 111, SEQ ID NO: 115, SEQ ID NO: 119 or SEQ ID NO: 123; (ii) a Domain II of a Cry protein from any of SEQ ID NO: 99, SEQ ID NO: 103, SEQ ID NO: 107, SEQ ID NO: 112, SEQ ID NO: 116, SEQ ID NO: 120 or SEQ ID NO: 124; and (iii) a Domain III of Cry protein from any of SEQ ID NO: 100, SEQ ID NO: 104, SEQ ID NO: 108, SEQ ID NO: 109, SEQ ID NO: 113, SEQ ID NO: 117, SEQ ID NO: 121 or SEQ ID NO: 125, in which the protein lacks a carboxy-terminal protoxin domain. Preferably, the truncated chimeric insecticidal protein comprises an amino acid sequence of any of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48, where the amino acid sequence lacks a carboxy-terminal protoxin domain. In another complementary embodiment, an expression cassette comprising a polynucleotide as defined above is provided.

In another embodiment, a host cell comprising the polynucleotide described above is provided in this document, in which the host cell is selected from the group consisting of a bacterial host cell or a plant host cell. Bacterial host cells included Agrobacterium, Rhizobium, Bacillus, Brevibacillus, Escherichia, Pseudomonas, Klebsiella, Erwinia, where the species Bacillus is a Bacillus cereus or a Bacillus thuringiensis, Brevibacillus is preferably Brevibacillus laterosperous, and Escherichia is preferably Escherichia coli. The plant cells considered include monocots and dicots.

Other embodiments provided in this document include insect inhibitor compositions comprising a truncated chimeric insecticidal protein comprising (i) a Cry protein Domain I of any of SEQ ID NO: 98, SEQ ID NO: 102, SEQ ID NO: 106, SEQ ID NO: 111, SEQ ID NO: 115, SEQ ID NO: 119 or SEQ ID NO: 123; (ii) a Domain II of a Cry protein from any of SEQ ID NO: 99, SEQ ID NO: 103, SEQ ID NO: 107, SEQ ID NO: 112, SEQ ID NO: 116, SEQ ID NO: 120 or SEQ ID NO: 124; and (iii) a Domain III of Cry protein from any of SEQ ID NO: 100, SEQ ID NO: 104, SEQ ID NO: 108, SEQ ID NO: 109, SEQ ID NO: 113, SEQ ID NO: 117, SEQ ID NO: 121 or SEQ ID NO: 125, in which the protein lacks a carboxy-terminal protoxin domain; preferably a truncated chimeric protein comprising the amino acid sequence of any of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48, where the amino acid sequence lacks a carboxy-terminal protoxin domain. In certain embodiments, the insect inhibitor composition further comprises at least one insect inhibitor agent different from the chimeric insecticidal protein. Insect inhibitors considered other than chimeric insecticidal protein include an insect inhibitor protein, an insect inhibitor dsRNA molecule, and an insect inhibitor chemistry. These insect inhibitors other than chimeric insecticidal protein may exhibit activity against one or more pest species of the orders Lepidoptera, Coleoptera, Hemiptera, Homoptera, or Thysanoptera.

In yet another embodiment disclosed in this document, a seed comprising an effective insect-inhibiting amount of: a chimeric insecticidal protein comprising (i) a Cry Protein Domain I of any of SEQ ID NO: 98, SEQ ID NO: 102, SEQ ID NO: 106, SEQ ID NO: 111, SEQ ID NO: 115, SEQ ID NO: 119 or SEQ ID NO: 123; (ii) a Domain II of a Cry protein from any of SEQ ID NO: 99, SEQ ID NO: 103, SEQ ID NO: 107, SEQ ID NO: 112, SEQ ID NO: 116, SEQ ID NO: 120 or SEQ ID NO: 124; and (iii) a Domain III of Cry protein from any of SEQ ID NO: 100, SEQ ID NO: 104, SEQ ID NO: 108, SEQ ID NO: 109, SEQ ID NO: 113, SEQ ID NO: 117, SEQ ID NO: 121 or SEQ ID NO: 125, in which the protein lacks a carboxy-terminal protoxin domain; preferably a truncated chimeric protein comprising the amino acid sequence of any of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48, where the amino acid sequence lacks a carboxy-terminal protoxin domain; or a polynucleotide comprising a sequence of nucleotides of any of SEQ ID NOs: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96, where the polynucleotide lacks a region coding for a carboxy-terminal protoxin domain.

Methods to control a Lepidopteran pest also include putting the Lepidopteran pest in contact with an inhibiting amount of a chimeric insecticidal protein of the invention.

In another embodiment, a plant genome comprising a nucleic acid molecule that encodes a chimeric insecticidal protein truncated according to the present invention or an expression cassette containing the said nucleic acid molecule is provided.

The present invention further provides a plant cell, plant, or part of the transgenic plant comprising a chimeric insecticidal protein or a nucleic acid molecule encoding the same, where: the truncated chimeric insecticidal protein comprises (i) a Cry protein Domain I of any of the SEQ ID NO: 98, SEQ ID NO: 102, SEQ ID NO: 106, SEQ ID NO: 111, SEQ ID NO: 115, SEQ ID NO: 119 or SEQ ID NO: 123; (ii) a Domain II of a Cry protein from any of SEQ ID NO: 99, SEQ ID NO: 103, SEQ ID NO: 107, SEQ ID NO: 112, SEQ ID NO: 116, SEQ ID NO: 120 or SEQ ID NO: 124; and one (iii) Domain III of Cry protein from any of SEQ ID NO: 100, SEQ ID NO: 104, SEQ ID NO: 108, SEQ ID NO: 109, SEQ ID NO: 113, SEQ ID NO: 117, SEQ ID NO: 121 or SEQ ID NO: 125, in which the protein lacks a carboxy-terminal protoxin domain; preferably a truncated chimeric protein comprising the amino acid sequence of any of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48, where the amino acid sequence lacks a carboxy-terminal protoxin domain.

In another embodiment, chimeric insecticidal protein is provided that is: at least 85% identical to SEQ ID NO: 1; at least 80% identical to SEQ ID NO: 2, at least 79% identical to SEQ ID NO: 3; at least 82% identical to SEQ ID NO: 4; at least 79% identical to SEQ ID NO: 5; At least 87% identical to SEQ ID NO: 6; at least 91% identical to SEQ ID NO: 7; At least 77% identical to SEQ ID NO: 8; at least 81% identical to SEQ ID NO: 9; at least 82% identical to SEQ ID NO: 10; at least 76% identical to SEQ ID NO: 11; at least 91% identical to SEQ ID NO: 12; at least 92% identical to SEQ ID NO: 13; At least 86% identical to SEQ ID NO: 14; at least 82% identical to SEQ ID NO: 15; At least 88% identical to SEQ ID NO: 16; at least 97% identical to SEQ ID NO: 17; at least 81% identical to SEQ ID NO:18; at least 86% identical to SEQ ID NO: 19; at least 76% identical to SEQ ID NO: 20; at least 78% identical to SEQ ID NO: 21; at least 78% identical to SEQ ID NO: 22; at least 78% identical to SEQ ID NO:23; at least 86% identical to SEQ ID NO:24; at least 85% identical to SEQ ID NO: 25; at least 83% identical to SEQ ID NO: 26; at least 83% identical to SEQ ID NO: 27, at least 85% identical to SEQ ID NO: 28, at least 79% identical to SEQ ID NO: 29, at least 85% identical to SEQ ID NO: 30, at least 80% identical to SEQ ID NO: 31, at least 84% identical to SEQ ID NO: 33, at least 78% identical to SEQ ID NO: 33, at least 85% identical to SEQ ID NO: 34, at least 80% identical to SEQ ID NO: 35, at least 87% identical to SEQ ID NO: 36, at least 93% identical to SEQ ID NO: 37, at least 84% identical to SEQ ID NO: 38, at least 81% identical to SEQ ID NO: 39, at least 80% identical to SEQ ID NO: 40, at least 84% identical to SEQ ID NO: 41, at least 93% identical to SEQ ID NO: 42, at least 86% identical to SEQ ID NO: 43, at least 70% identical to SEQ ID NO: 44, at least 75% identical to SEQ ID NO: 45, at least 73% identical to SEQ ID NO: 46, at least 50% identical to SEQ ID NO: 47, or at least 50% identical to SEQ ID NO: 48, where the amino acid sequence lacks a carboxy-terminal protoxin domain. Nucleic acid molecules encoding the aforementioned proteins, expression cassettes, plant cells, plants, plant parts, and seeds containing a recombinant nucleic acid molecule encoding one or more of the chimeric proteins provided in this document are also supplied.

Methods of controlling a Lepidopteran pest are also provided that involve exposing the pest to a truncated chimeric protein described in this document, plant cell, plant, or part of the transgenic plant, in which the said plant cell, plant, or part of the plant expresses a truncated chimeric protein described in this document.

In other embodiments described in this document, plant products derived from plant cell, plant, or part of a plant are provided, in which the product comprises a detectable amount of the chimeric insecticidal protein. The plant products included plant biomass, oil, bran, animal feed, flour, flakes, husks, and processed seeds.

Another method described here is a method of producing a seed comprising the chimeric insecticidal protein, the method comprising: planting at least one seed that includes the chimeric insecticidal protein; growing plants from the said seed; and harvesting seeds from the plants, wherein the harvested seed comprises the chimeric insecticidal protein.

The recombinant polynucleotide molecules encoding a truncated chimeric insecticidal protein comprise a nucleotide sequence of any of SEQ ID NOs: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96, where the polynucleotide lacks a region coding for a carboxy-terminal protoxin domain; and, optionally, a polynucleotide sequence encoding an insect inhibitor agent different from the chimeric insecticidal protein are also contemplated in this document.

Another recombinant nucleic acid molecule contemplated in this document comprises a heterologous promoter operationally linked to a polynucleotide segment encoding chimeric insecticidal proteins, where: the truncated chimeric insecticidal protein comprises (i) a Cry protein Domain I of any of the SEQ ID NO: 98, SEQ ID NO: 102, SEQ ID NO: 106, SEQ ID NO: 111, SEQ ID NO: 115, SEQ ID NO: 119 or SEQ ID NO: 123; (ii) a Domain II of a Cry protein from any of SEQ ID NO: 99, SEQ ID NO: 103, SEQ ID NO: 107, SEQ ID NO: 112, SEQ ID NO: 116, SEQ ID NO: 120 or SEQ ID NO: 124; and one (iii) Domain III of Cry protein from any of SEQ ID NO: 100, SEQ ID NO: 104, SEQ ID NO: 108, SEQ ID NO: 109, SEQ ID NO: 113, SEQ ID NO: 117, SEQ ID NO: 121 or SEQ ID NO: 125, in which the protein lacks a carboxy-terminal protoxin domain; or the truncated chimeric insecticidal protein comprises a protein that possesses: at least 85% identical to SEQ ID NO: 1; at least 80% identical to SEQ ID NO: 2, at least 79% identical to SEQ ID NO: 3; at least 82% identical to SEQ ID NO: 4; at least 79% identical to SEQ ID NO: 5; at least 87% identical to SEQ ID NO: 6; at least 91% identical to SEQ ID NO: 7; At least 77% identical to SEQ ID NO: 8; at least 81% identical to SEQ ID NO: 9; at least 82% identical to SEQ ID NO: 10; at least 76% identical to SEQ ID NO: 11; at least 91% identical to SEQ ID NO: 12; at least 92% identical to SEQ ID NO: 13; at least 86% identical to SEQ ID NO: 14; at least 82% identical to SEQ ID NO: 15; At least 88% identical to SEQ ID NO: 16; at least 97% identical to SEQ ID NO: 17; at least 81% identical to SEQ ID NO:18; at least 86% identical to SEQ ID NO: 19; at least 76% identical to SEQ ID NO: 20; at least 78% identical to SEQ ID NO: 21; at least 78% identical to SEQ ID NO: 22; at least 78% identical to SEQ ID NO:23; at least 86% identical to SEQ ID NO:24; at least 85% identical to SEQ ID NO: 25; at least 83% identical to SEQ ID NO: 26; at least 83% identical to SEQ ID NO: 27, at least 85% identical to SEQ ID NO: 28, at least 79% identical to SEQ ID NO: 29, at least 85% identical to SEQ ID NO: 30, at least 80% identical to SEQ ID NO: 31, at least 84% identical to SEQ ID NO: 33, at least 78% identical to SEQ ID NO: 33, at least 85% identical to SEQ ID NO: 34, at least 80% identical to SEQ ID NO: 35, at least 87% identical to SEQ ID NO: 36, at least 93% identical to SEQ ID NO: 37, at least 84% identical to SEQ ID NO: 38, at least 81% identical to SEQ ID NO: 39, at least 80% identical to SEQ ID NO: 40, at least 84% identical to SEQ ID NO: 41, at least 93% identical to SEQ ID NO: 42, at least 86% identical to SEQ ID NO: 43, at least 70% identical to SEQ ID NO: 44, at least 75% identical to SEQ ID NO: 45, at least 73% identical to SEQ ID NO: 46, at least 50% identical to SEQ ID NO: 47, or at least 50% identical to SEQ ID NO: 48; or the polynucleotide segment hybridizes with a polynucleotide that has a nucleotide sequence of any of the SEQ ID NOs: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96, where the polynucleotide lacks a region encoding a carboxy-terminal protoxin domain.

Other embodiments, features and advantages of the invention will become evident from the following detailed description, examples and claims.

### BRIEF DESCRIPTION OF THE SEQUENCES

SEQ ID NO: 1 is the amino acid sequence of the chimeric protein truncated EMS_Q1.

SEQ ID NO: 2 is the amino acid sequence of the chimeric protein truncated EMS_Q2.

SEQ ID NO: 3 is the amino acid sequence of the truncated chimeric protein EMS_Q3.

SEQ ID NO: 4 is the amino acid sequence of the chimeric protein truncated EMS_Q4.

SEQ ID NO: 5 is the amino acid sequence of the chimeric protein truncated EMS_Q5.

SEQ ID NO: 6 is the amino acid sequence of the chimeric protein truncated EMS_Q6.

SEQ ID NO: 7 is the amino acid sequence of the chimeric protein truncated EMS_Q7.

SEQ ID NO: 8 is the amino acid sequence of the chimeric protein truncated EMS_Q8.

SEQ ID NO: 9 is the amino acid sequence of the chimeric protein truncated EMS_Q9.

SEQ ID NO: 10 is the amino acid sequence of the chimeric protein truncated EMS_Q10.

SEQ ID NO: 11 is the amino acid sequence of the truncated chimeric protein EMS_Q11.

SEQ ID NO: 12 is the amino acid sequence of the chimeric protein truncated EMS_Q12.

SEQ ID NO: 13 is the amino acid sequence of the truncated chimeric protein EMS_Q13.

SEQ ID NO: 14 is the amino acid sequence of the chimeric protein truncated EMS_Q14.

SEQ ID NO: 15 is the amino acid sequence of the truncated chimeric protein EMS_Q15.

SEQ ID NO: 16 is the amino acid sequence of the chimeric protein truncated EMS_Q16.

SEQ ID NO: 17 is the amino acid sequence of the truncated chimeric protein EMS_Q17.

SEQ ID NO: 18 is the amino acid sequence of the truncated chimeric protein EMS_Q18.

SEQ ID NO: 19 is the amino acid sequence of the truncated chimeric protein EMS_Q19.

SEQ ID NO: 20 is the amino acid sequence of the chimeric protein truncated EMS_Q20.

SEQ ID NO: 21 is the amino acid sequence of the truncated chimeric protein EMS_Q21.

SEQ ID NO: 22 is the amino acid sequence of the truncated chimeric protein EMS_Q22.

SEQ ID NO: 23 is the amino acid sequence of the truncated chimeric protein EMS_Q23.

SEQ ID NO: 24 is the amino acid sequence of the chimeric protein truncated EMS_Q24.

SEQ ID NO: 25 is the amino acid sequence of the truncated chimeric protein EMS_Q25.

SEQ ID NO: 26 is the amino acid sequence of the chimeric protein truncated EMS_Q26.

SEQ ID NO: 27 is the amino acid sequence of the truncated chimeric protein EMS_Q27.

SEQ ID NO: 28 is the amino acid sequence of the truncated chimeric protein EMS_Q28.

SEQ ID NO: 29 is the amino acid sequence of the truncated chimeric protein EMS_Q29.

SEQ ID NO: 30 is the amino acid sequence of the chimeric protein truncated EMS_Q30.

SEQ ID NO: 31 is the amino acid sequence of the truncated chimeric protein EMS_Q31.

SEQ ID NO: 32 is the amino acid sequence of the chimeric protein truncated EMS_Q32.

SEQ ID NO: 33 is the amino acid sequence of the truncated chimeric protein EMS_Q33.

SEQ ID NO: 34 is the amino acid sequence of the truncated chimeric protein EMS_Q34.

SEQ ID NO: 35 is the amino acid sequence of the truncated chimeric protein EMS_Q35.

SEQ ID NO: 36 is the amino acid sequence of the truncated chimeric protein EMS_Q36.

SEQ ID NO: 37 is the amino acid sequence of the truncated chimeric protein EMS_Q37.

SEQ ID NO: 38 is the amino acid sequence of the truncated chimeric protein EMS_Q38.

SEQ ID NO: 39 is the amino acid sequence of the truncated chimeric protein EMS_Q39.

SEQ ID NO: 40 is the amino acid sequence of the truncated chimeric protein EMS_Q40.

SEQ ID NO: 41 is the amino acid sequence of the truncated chimeric protein EMS_Q41.

SEQ ID NO: 42 is the amino acid sequence of the chimeric protein truncated EMS_Q42.

SEQ ID NO: 43 is the amino acid sequence of the truncated chimeric protein EMS_Q43.

SEQ ID NO: 44 is the amino acid sequence of the truncated chimeric protein EMS_Q44.

SEQ ID NO: 45 is the amino acid sequence of the chimeric protein truncated EMS_Q45.

SEQ ID NO: 46 is the amino acid sequence of the truncated chimeric protein EMS_Q46.

SEQ ID NO: 47 is the amino acid sequence of the truncated chimeric protein EMS_Q47.

SEQ ID NO: 48 is the amino acid sequence of the truncated chimeric protein EMS_Q48.

SEQ ID NO: 49 is the sequence of nucleotides that encodes the chimeric truncated protein EMS_1.

SEQ ID NO: 50 is the sequence of nucleotides that encodes the truncated chimeric protein EMS_2.

SEQ ID NO: 51 is the sequence of nucleotides that encodes the truncated chimeric protein EMS_3.

SEQ ID NO: 52 is the sequence of nucleotides that encodes the truncated chimeric protein EMS_4.

SEQ ID NO: 53 is the nucleotide sequence that encodes the truncated chimeric protein EMS_5.

SEQ ID NO: 54 is the sequence of nucleotides that encodes the chimeric truncated protein EMS_6.

SEQ ID NO: 55 is the sequence of nucleotides that encodes the truncated chimeric protein EMS_7.

SEQ ID NO: 56 is the nucleotide sequence that encodes the truncated chimeric protein EMS_8.

SEQ ID NO: 57 is the sequence of nucleotides that encodes the truncated chimeric protein EMS_9.

SEQ ID NO: 58 is the sequence of nucleotides that encodes the truncated chimeric protein EMS_10.

SEQ ID NO: 59 is the nucleotide sequence that encodes the truncated chimeric protein EMS_11.

SEQ ID NO: 60 is the sequence of nucleotides that encodes the truncated chimeric protein EMS_12.

SEQ ID NO: 61 is the sequence of nucleotides that encodes the truncated chimeric protein EMS_13.

SEQ ID NO: 62 is the sequence of nucleotides that encodes the truncated chimeric protein EMS_14.

SEQ ID NO: 63 is the sequence of nucleotides that encodes the truncated chimeric protein EMS_15.

SEQ ID NO: 64 is the sequence of nucleotides that encodes the chimeric truncated protein EMS_16.

SEQ ID NO: 65 is the nucleotide sequence that encodes the truncated chimeric protein EMS_17.

SEQ ID NO: 66 is the sequence of nucleotides that encodes the chimeric truncated protein EMS_18.

SEQ ID NO: 67 is the sequence of nucleotides that encodes the truncated chimeric protein EMS_19.

SEQ ID NO: 68 is the sequence of nucleotides that encodes the truncated chimeric protein EMS_20.

SEQ ID NO: 69 is the sequence of nucleotides that encodes the truncated chimeric protein EMS_21.

SEQ ID NO: 70 is the nucleotide sequence that encodes the truncated chimeric protein EMS_22.

SEQ ID NO: 71 is the sequence of nucleotides that encodes the truncated chimeric protein EMS_23.

SEQ ID NO: 72 is the sequence of nucleotides that encodes the truncated chimeric protein EMS_24.

SEQ ID NO: 73 is the nucleotide sequence that encodes the truncated chimeric protein EMS_25.

SEQ ID NO: 74 is the nucleotide sequence that encodes the chimeric truncated protein EMS_26.

SEQ ID NO: 75 is the sequence of nucleotides that encodes the chimeric protein truncated EMS_27.

SEQ ID NO: 76 is the sequence of nucleotides that encodes the truncated chimeric protein EMS_28.

SEQ ID NO: 77 is the sequence of nucleotides that encodes the chimeric protein truncated EMS_29.

SEQ ID NO: 78 is the sequence of nucleotides that encodes the truncated chimeric protein EMS_30.

SEQ ID NO: 79 is the nucleotide sequence that encodes the truncated chimeric protein EMS_31.

SEQ ID NO: 80 is the sequence of nucleotides that encodes the truncated chimeric protein EMS_32.

SEQ ID NO: 81 is the nucleotide sequence that encodes the truncated chimeric protein EMS_33.

SEQ ID NO: 82 is the nucleotide sequence that encodes the EMS_34 truncated chimeric protein.

SEQ ID NO: 83 is the sequence of nucleotides that encodes the chimeric truncated protein EMS_35.

SEQ ID NO: 84 is the sequence of nucleotides that encodes the chimeric truncated protein EMS_36.

SEQ ID NO: 85 is the sequence of nucleotides that encodes the truncated chimeric protein EMS_37.

SEQ ID NO: 86 is the sequence of nucleotides that encodes the truncated chimeric protein EMS_38.

SEQ ID NO: 87 is the sequence of nucleotides that encodes the truncated chimeric protein EMS_39.

SEQ ID NO: 88 is the sequence of nucleotides that encodes the truncated chimeric protein EMS_40.

SEQ ID NO: 89 is the sequence of nucleotides that encodes the truncated chimeric protein EMS_41.

SEQ ID NO: 90 is the sequence of nucleotides that encodes the chimeric truncated protein EMS_42.

SEQ ID NO: 91 is the nucleotide sequence that encodes the chimeric truncated protein EMS_43.

SEQ ID NO: 92 is the nucleotide sequence that encodes the truncated chimeric protein EMS_44.

SEQ ID NO: 93 is the nucleotide sequence that encodes the truncated chimeric protein EMS_45.

SEQ ID NO: 94 is the nucleotide sequence that encodes the truncated chimeric protein EMS_46.

SEQ ID NO: 95 is the nucleotide sequence that encodes the truncated chimeric protein EMS_47.

SEQ ID NO: 96 is the sequence of nucleotides that encodes the truncated chimeric protein EMS_48.

SEQ ID NO: 97 is the amino acid sequence of the parental protein Cry1C.

SEQ ID NO: 98 is the amino acid sequence of Domain I of Cry1C.

SEQ ID NO: 99 is the amino acid sequence of Domain II of Cry1C.

SEQ ID NO: 100 is the amino acid sequence of Domain III of Cry1C.

SEQ ID NO: 101 is the amino acid sequence of the parent protein Cry1B.

SEQ ID NO: 102 is the amino acid sequence of Domain I of Cry1B.

SEQ ID NO: 103 is the amino acid sequence of Domain II of Cry1B.

SEQ ID NO: 104 is the amino acid sequence of Domain III of Cry1B.

SEQ ID NO: 105 is the amino acid sequence of the parental protein Cry1Ab.

SEQ ID NO: 106 is the amino acid sequence of Domain I of Cry1Ab.

SEQ ID NO: 107 is the amino acid sequence of Domain II of Cry1Ab.

SEQ ID NO: 108 is the amino acid sequence of the III_a Domain of Cry1Ab.

SEQ ID NO: 109 is the amino acid sequence of the III_b Domain of Cry1Ab.

SEQ ID NO: 110 is the amino acid sequence of the parent protein Cry1E.

SEQ ID NO: 111 is the amino acid sequence of Domain I of Cry1E.

SEQ ID NO: 112 is the amino acid sequence of Domain II of Cry1E.

SEQ ID NO: 113 is the amino acid sequence of Domain III of Cry1E.

SEQ ID NO: 114 is the amino acid sequence of the parental protein Cry1 Da.

SEQ ID NO: 115 is the amino acid sequence of Domain I of Cry1 Da.

SEQ ID NO: 116 is the amino acid sequence of Domain II of Cry1 Da.

SEQ ID NO: 117 is the amino acid sequence of Domain III of Cry1 Da.

SEQ ID NO: 118 is the amino acid sequence of the parental protein Cry1Fb.

SEQ ID NO: 119 is the amino acid sequence of Domain I of Cry1Fb.

SEQ ID NO: 120 is the amino acid sequence of Domain II of Cry1Fb.

SEQ ID NO: 121 is the amino acid sequence of Domain III of Cry1Fb.

SEQ ID NO: 122 is the amino acid sequence of the parent protein Cry2Aa.

SEQ ID NO: 123 is the amino acid sequence of Domain I of Cry2Aa.

SEQ ID NO: 124 is the amino acid sequence of Domain II of Cry2Aa.

SEQ ID NO: 125 is the amino acid sequence of Domain III of Cry2Aa.

### BRIEF DESCRIPTION OF THE FIGURES

FIGS. 1-4 show the results of the trials reported in Example 5.

FIG. 1: Graphical result of the mean injury score (Davis scale) between 3 evaluations (7, 14, and 21 days after infestation) of the greenhouse assay with artificial infestation of Spodoptera frugiperda in 44 pots with corn events containing the chimeric protein coding sequence EMS_Q6 (SEQ ID NO: 6). Non-transgenic corn L3 was added as a negative control (C−), and a commercial transgenic corn for resistance to S. frugiperda was added as a positive control (C+).

FIG. 2. Graphical result of the mean injury score (Davis scale) between 3 evaluations (7, 14, and 21 days after infestation) of the field trial with artificial infestation of S. frugiperda in 22 events (Ev1 to Ev22) of corn containing the chimeric protein coding sequence EMS_Q6 (SEQ ID NO: 6). As negative and positive controls, two non-transgenic corns (C−) and two commercial transgenic corns (C+) were evaluated, respectively.

FIG. 3: Representative result of the field trial with artificial and natural infestation of S. frugiperda in (A) transgenic corn, expressing chimeric protein EMS_Q6 (SEQ ID NO: 6) and (B) non-transgenic corn control. It is noted that the transgenic corn plant does not present damage, while the conventional corn plant shows leaf damage caused by the attack of S. frugiperda.

FIG. 4: Result of the bioassay conducted in the laboratory with corn leaves after five days of infestation with neonatal larvae of S. frugiperda. (A) transgenic corn, expressing chimeric protein EMS_Q6 (SEQ ID NO: 6) and (B) non-transgenic control corn (C−). It is noted that in the corn leaf expressing the EMS_Q6 protein, there was total mortality of the caterpillars and absence of leaf damage. In the control corn, there was evident damage from feeding and the presence of live and well-developed caterpillars.

### DETAILED DESCRIPTION OF THE INVENTION

The problem in the agricultural insect pest control technique can be defined as a need for new insecticidal proteins that are effective against target pest insects, exhibit broad-spectrum toxicity against target pest insect species, have the ability to be expressed in plants without causing undesirable agronomic problems, and provide an alternative mode of action compared to proteins currently used in agriculture. The truncated chimeric insecticidal proteins disclosed in this document address each of these needs, particularly against a broad spectrum of Lepidopteran insect pests.

In order to prevent the development of, or circumvent insect resistance against currently used insecticidal proteins, new insecticidal proteins with different modes of action, as well as a broad spectrum and efficacy, are necessary for the control of Lepidoptera. One way to address this need is to exchange segments between several Bt proteins that exhibit structural similarities to create new Bt proteins with insect-inhibiting properties. However, the probability of creating a chimeric protein with improved properties, from the variety of domain structures of the numerous native insecticidal proteins known in the technique, is remote. See, e.g., Jacqueline S. Knight, et al. “A Strategy for Shuffling Numerous Bacillus thuringiensis Crystal Protein Domains”. J. Economic Entomology, 97 (6) (2004): pages 1805 to 1813.

Sequences of recombinant nucleic acid molecules encoding new chimeric insecticidal proteins are disclosed in this document. These insecticidal proteins address the ongoing need in the technique for the provision of additional toxic insecticidal proteins with improved insecticidal properties such as increased efficacy, activity against a broader spectrum of target insect pest species, and different modes of action. Members of this group of proteins, which include the proteins exemplified and provided in this document, exhibit insecticidal activity against Lepidopteran insect pest species.

The term “segment” or “fragment” is used in this application to describe consecutive amino acid or nucleic acid sequences that are shorter than the full amino acid or nucleic acid sequence describing a disclosed chimeric insecticidal protein. A segment or fragment exhibiting insect-inhibiting activity is also disclosed in the present application if the alignment of such segment or fragment with the corresponding section of the chimeric insecticidal protein results in amino acid sequence identity of any fraction percentage from about 50% to about 100% between the segment or fragment and the corresponding section of the chimeric insecticidal protein.

The reference in this application to the terms “active” or “activity”; “pesticidal activity” or “pesticide”; or “insecticidal activity”, “insect inhibitor”, “insecticide”, refers to the efficacy of a toxic agent, such as an insecticidal protein, in inhibiting (inhibiting growth, feeding, fecundity, or viability), suppressing (suppressing pest infestation, suppressing pest feeding activities in a particular crop containing an effective amount of an insecticidal protein), or killing (causing morbidity, mortality, or reduced fecundity) of a pest. These terms are intended to include the result of providing an effective amount of an insecticidal protein to a pest in which exposure of the pest to the insecticidal protein results in morbidity, mortality, reduced fecundity, or growth retardation. These terms also include the repulsion of the plant pest, from a plant tissue, a part of the plant, the seed, plant cells, or the particular geographic location where the plant may be being cultivated, as a result of providing an effective amount of the insecticidal protein as a pesticide in or on the plant. Generally speaking, pesticidal activity refers to the ability of an insecticidal protein to be effective in inhibiting the growth, development, viability, feeding behavior, mating behavior, fecundity, or any measurable decrease in the adverse effects caused by insect feeding on this protein, protein fragment, protein segment, or polynucleotide of a particular target pest, including, without limitation, insects of the order Lepidoptera. The insecticidal protein can be produced by the plant or it can be applied to the plant or the environment within the location where the plant is located. The term “bioactivity,” “efficacy,” or variations thereof are also terms used interchangeably in this application to describe the effects of the truncated chimeric insecticidal proteins of the present invention on target insect pests.

The terms “truncated” or “truncated” refer to amino acid sequences of proteins that, compared to the corresponding natural and/or parental sequences, lack the carboxy-terminal portion of protoxin present in the natural and/or parental Cry1 and Cry2 proteins. The terms “truncated” or “truncated” further refer to nucleotide sequences that encode truncated proteins and, therefore, lack a region coding for a carboxy-terminal protoxin domain.

A pesticide-effective amount of a toxic agent, when provided in the diet of a target pest, exhibits pesticidal activity when the toxic agent comes into contact with the pest. A toxic agent can be an insecticidal protein or one or more chemical agents known in the art. Insecticidal chemical agents and insecticidal protein agents can be used alone or in combination with each other. Chemical agents include, but are not limited to, dsRNA molecules targeting specific genes for the suppression of a target pest, organochlorides, organophosphates, carbamates, pyrethroids, neonicotinoids, and ryanoids. Insecticidal protein agents include the chimeric insecticidal proteins presented in this application, as well as other toxic protein agents, including those that target lepidopteran pest species, as well as protein toxins that are used to control other plant pests, such as Cry proteins available in the technique for use in the control of species of Coleoptera, Thysanoptera, Hemiptera, and Homoptera.

The reference to a pest, particularly an agricultural pest, means crop insect pests, particularly Lepidopteran insect pests, which are controlled by the truncated chimeric insecticidal proteins disclosed in this document. However, the reference to a pest may also include pests of plant Coleopteran, Hemipteran, and Homopteran insects, as well as nematodes, when toxic agents against these pests are combined or are present together with the truncated chimeric insecticidal protein or with a protein that is 50% to about 100% identical to the truncated chimeric insecticidal protein.

The truncated chimeric insecticidal proteins described in this document exhibit insecticidal activity against insect pests from Lepidoptera species, including adults, pupae, larvae, and newborns, as well as Hemiptera species, including adults and nymphs. Insects of the order Lepidoptera include, but are not limited to, Agrotis ipsilon, Agrotis subterranea, Agrotis orthogonia, Alabama argillacea, Anticarsia gemmatalis, Amyelois transitella, Archips argyrospila, Archips rosana, Chilo suppressalis, Chrysodeixis includens, Cnaphalocrocis medinalis, Crambus caliginosellus, Crambus teterrellus, Cydia pomonella, Diatraea saccharalis, Diatraea grandiosella, Earias vitela, Earias insulana, Earias vittella, Elasmopalpus lignosellus, Endopiza viteana, Grapholita molesta, Helicoverpa armigera, Helicoverpa gelotopeon, Helicoperva zea, Heliothis virescens, Herpetogramma licarsisalis, Homoeosoma electellum, Hypena scabra, Lobesia botrana, Lymantria dispar, Mamestra configurata, Ostrinia nubilalis, Pectinophora gossypiella, Pieris brassicae, Pieris rapae, Phyllocnistis citrella, Plutella xylostella, Pseudaletia unipuncta, Rachiplusia nu, Sesamia inferens, Spodoptera cosmioides, Spodoptera exigua, Spodoptera eridania, Spodoptera frugiperda, Spodoptera litura, Suleima helianthana, Trichoplusia ni, and Tuta absoluta.

Reference in this application to an “isolated DNA molecule,” or an equivalent term or phrase, means that the DNA molecule is present alone or in combination with other compositions, but not within its natural environment. For example, nucleic acid elements, such as a coding sequence, intron sequence, untranslated main sequence, promoter sequence, transcription termination sequence, and the like, that are found naturally in the DNA of an organism's genome are not considered to be “isolated” since the element is within the organism's genome and at the location within the genome in which it is naturally found. However, each of these elements, and subparts of these elements, would be “isolated” within the scope of this document, as long as the element is not within the genome of the organism and in the place within the genome in which it is naturally found. Similarly, a nucleotide sequence that encodes an insecticidal protein or any naturally occurring insecticidal variant of that protein would be a solitary nucleotide sequence, provided that the nucleotide sequence is not within the DNA of the bacterium from which the sequence encoding the protein is naturally found. A sequence of synthetic nucleotides encoding the amino acid sequence of the naturally occurring insecticidal protein would be considered isolated for the purposes of this document. For the purposes of this document, any recombinant nucleotide sequence, for example, the DNA nucleotide sequence inserted into the genome of the cells of a plant or bacterium, or present in an extrachromosomal vector, would be considered an isolated nucleotide sequence if it is present within the plasmid or similar structure used to transform the cells, within the genome of the plant or bacterium, or present in detectable amounts in tissues, offspring, biological samples, or consumer goods products derived from the plant or bacterium.

As further described in the Examples, through a chimeric sequence development effort, about forty-eight (48) nucleotide sequences encoding truncated chimeric insecticidal proteins were constructed from the toxin domains of known insecticidal proteins (referred to in this document as “parental proteins”), and expressed and tested in the bioassay for activity against Lepidoptera. Truncated chimeric proteins constructed according to the teachings of this document showed superior activity in the control of Lepidoptera compared to the progenitor proteins from which their toxin components were derived.

These chimeric insecticidal proteins with enhanced Lepidoptera activity or an improved Lepidoptera spectrum were constructed from the following amino-terminal toxin domains of Bt parental proteins Cry1Ab, Cry1B, Cry1C, Cry1 Da, Cry1E, Cry1Fb, and Cry2Aa. Specifically, the novel chimeric insecticidal proteins of the present invention with enhanced Lepidoptera activity or an improved Lepidoptera spectrum comprise the following domain combinations: Cry1C Domain I (SEQ ID NO: 98), Cry1B (SEQ ID NO: 102), Cry1Ab (SEQ ID NO: 106), Cry1E (SEQ ID NO: 111), Cry1 Da (SEQ ID NO: 115), Cry1 Fb (SEQ ID NO: 119), Cry2Aa (SEQ ID NO: 123); Cry1C Domain II (SEQ ID NO: 99), Cry1B (SEQ ID NO: 103), Cry1Ab (SEQ ID NO: 107), Cry1E (SEQ ID NO: 112), Cry1 Da (SEQ ID NO: 116), Cry1Fb (SEQ ID NO: 120), Cry2Aa (SEQ ID NO: 124); and Domain III of Cry1C (SEQ ID NO: 100), Cry1B (SEQ ID NO: 104), Cry1Ab_a (SEQ ID NO: 108), Cry1Ab_b (SEQ ID NO: 109), Cry1E (SEQ ID NO: 113), Cry1 Da (SEQ ID NO: 117), Cry1Fb (SEQ ID NO: 121), Cry2Aa (SEQ ID NO: 125).

Exemplary truncated chimeric proteins according to the present invention have been named EMS_Q1 (SEQ ID NO: 1), EMS_Q2 (SEQ ID NO: 2), EMS_Q3 (SEQ ID NO: 3), EMS_Q4 (SEQ ID NO: 4), EMS_Q5 (SEQ ID NO: 5), EMS_Q6 (SEQ ID NO: 6), EMS_Q7 (SEQ ID NO: 7), EMS_Q8 (SEQ ID NO: 8), EMS_Q9 (SEQ ID NO: 9), EMS_Q10 (SEQ ID NO: 10), EMS_Q11 (SEQ ID NO: 11), EMS_Q12 (SEQ ID NO: 12), EMS_Q13 (SEQ ID NO: 13), EMS_Q14 (SEQ ID NO: 14), EMS_Q15 (SEQ ID NO: 15), EMS_Q16 (SEQ ID NO: 16), EMS_Q17 (SEQ ID NO: 17), EMS_Q18 (SEQ ID NO: 18), EMS_Q19 (SEQ ID NO: 19), EMS_Q20 (SEQ ID NO: 20), EMS_Q21 (SEQ ID NO: 21), EMS_Q22 (SEQ ID NO: 22), EMS_Q23 (SEQ ID NO: 23), EMS_Q24 (SEQ ID NO: 24), EMS_Q25 (SEQ ID NO: 25), EMS_Q26 (SEQ ID NO: 26), EMS_Q27 (SEQ ID NO: 27), EMS_Q28 (SEQ ID NO: 28), EMS_Q29 (SEQ ID NO: 29), EMS_Q30 (SEQ ID NO: 30), EMS_Q31 (SEQ ID NO: 31), EMS_Q32 (SEQ ID NO: 32), EMS_Q33 (SEQ ID NO: 33), EMS_Q34 (SEQ ID NO: 34), EMS_Q35 (SEQ ID NO: 35), EMS_Q36 (SEQ ID NO: 36), EMS_Q37 (SEQ ID NO: 37), EMS_Q38 (SEQ ID NO: 38), EMS_Q39 (SEQ ID NO: 39), EMS_Q40 (SEQ ID NO: 40), EMS_Q41 (SEQ ID NO: 41), EMS_Q42 (SEQ ID NO: 42), EMS_Q43 (SEQ ID NO: 43), EMS_Q44 (SEQ ID NO: 44), EMS_Q45 (SEQ ID NO: 45), EMS_Q46 (SEQ ID NO: 46), EMS_Q47 (SEQ ID NO: 47), and EMS_Q48 (SEQ ID NO: 48).

Many of the truncated chimeric insecticidal proteins demonstrate insecticidal activity against multiple species of Lepidopteran insect pests. Specifically, the truncated chimeric insecticidal proteins described in this application exhibited activity against S. frugiperda. Thus, the exemplified proteins described in this application are related by common function and exhibit insecticidal activity against insect pests from Lepidopteran insect species, including adults, larvae, and pupae.

The proteins that chimeric insecticides can be identified by comparison with each other using various computer-based algorithms known in the technique. For example, the amino acid sequence identity of proteins related to truncated chimeric insecticidal proteins can be analyzed using a Clustal W alignment with these standard parameters: weight matrix: blosum, gap opening penalty: 10.0, gap extension penalty: 0.05, hydrophilic gaps: Bonded, hydrophilic residues: GPSNDQERK, Specific Residue Gap Penalties: Linked (Thompson, et al (1994) Nucleic Acids Research, 22: 4,673 to 4,680). The percentage of amino acid identity is further calculated by the product of 100% multiplied by (amino acid identities/length of the protein in question). Other alignment algorithms are also available in the technique, provide results similar to those obtained using a Clustal W alignment, and are contemplated in this application.

A query protein that exhibits insect inhibitory activity is described in this application if the alignment of such query protein with the truncated chimeric insecticidal proteins established in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 and results in at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100% amino acid sequence identity (or any fraction percentage in this range) between the query protein and matter.

As further described in the examples of this application, synthetic or artificial sequences encoding chimeric insecticidal proteins have been designed for use in plants. Exemplary synthetic nucleotide sequences that have been designed for use in plants established in the fields are EMS_Q1 (SEQ ID NO: 49), EMS_Q2 (SEQ ID NO: 50), EMS_Q3 (SEQ ID NO: 51), EMS_Q4 (SEQ ID NO: 52), EMS_Q5 (SEQ ID NO: 53), EMS_Q6 (SEQ ID NO: 54), EMS_Q7 (SEQ ID NO: 55), EMS_Q8 (SEQ ID NO: 56), EMS_Q9 (SEQ ID NO: 57), EMS_Q10 (SEQ ID NO: 58), EMS_Q11 (SEQ ID NO: 59), EMS_Q12 (SEQ ID NO: 60), EMS_Q13 (SEQ ID NO: 61), EMS_Q14 (SEQ ID NO: 62), EMS_Q15 (SEQ ID NO: 63), EMS_Q16 (SEQ ID NO: 64), EMS_Q17 (SEQ ID NO: 65), EMS_Q18 (SEQ ID NO: 66), EMS_Q19 (SEQ ID NO: 67), EMS_Q20 (SEQ ID NO: 68), EMS_Q21 (SEQ ID NO: 69), EMS_Q22 (SEQ ID NO: 70), EMS_Q23 (SEQ ID NO: 71), EMS_Q24 (SEQ ID NO: 72), EMS_Q25 (SEQ ID NO: 73), EMS_Q26 (SEQ ID NO: 74), EMS_Q27 (SEQ ID NO: 75), EMS_Q28 (SEQ ID NO: 76), EMS_Q29 (SEQ ID NO: 77), EMS_Q30 (SEQ ID NO: 78), EMS_Q31 (SEQ ID NO: 79), EMS_Q32 (SEQ ID NO: 80), EMS_Q33 (SEQ ID NO: 81), EMS_Q34 (SEQ ID NO: 82), EMS_Q35 (SEQ ID NO: 83), EMS_Q36 (SEQ ID NO: 84), EMS_Q37 (SEQ ID NO: 85), EMS_Q38 (SEQ ID NO: 86), EMS_Q39 (SEQ ID NO: 87), EMS_Q40 (SEQ ID NO: 88), EMS_Q41 (SEQ ID NO: 89), EMS_Q42 (SEQ ID NO: 90), EMS_Q43 (SEQ ID NO: 91), EMS_Q44 (SEQ ID NO: 92), EMS_Q45 (SEQ ID NO: 93), EMS_Q46 (SEQ ID NO: 94), EMS_Q47 (SEQ ID NO: 95), and EMS_Q48 (SEQ ID NO: 96).

For expression in plant cells, chimeric insecticidal proteins can be expressed to reside in the cytosol or directed to various organelles of the plant cell. For example, targeting a protein to the chloroplast can result in increased levels of protein expressed in a transgenic plant, preventing different phenotypes from occurring. Targeting can also result in an increase in the effectiveness of pest resistance in a transgenic event. A target peptide or transit peptide is a short peptide chain (3 to 70 amino acids in length) that directs the transport of a protein to a specific region of the cell, including the nucleus, mitochondria, endoplasmic reticulum (ER), chloroplasts, apoplast, peroxisome, and plasma membrane. Some transit peptides are cleaved from the protein by peptidase after the proteins are transported. For targeting to the chloroplast, proteins contain transit peptides that have about 40 to 50 amino acids. Description of the use of chloroplast transit peptides is provided, for example in WO2013116758, U.S. Pat. Nos. 5,188,642, 5,728,925, and 9,150,625. Many proteins located in chloroplasts are expressed from nuclear genes as precursors and are directed to the chloroplast by a chloroplast transit peptide (CTP). Examples of such isolated chloroplast proteins include, but are not limited to, those associated with the small subunit (SSU) of ribulose-1,5-bisphosphate carboxylase, ferredoxin, ferredoxin oxidoreductase, the light-harvesting complex I and protein II, thioredoxin F, and enolpyruvyl shikimate phosphate synthase (EPSPS). It has been demonstrated in vivo and in vitro that proteins not expressed in the chloroplast can be directed to the chloroplast by using protein fusions with a heterologous CTP, and that the CTP is sufficient to target a protein to the chloroplast. It has been shown that the incorporation of a suitable chloroplast transit peptide such as EPSPS CTP (CTP2) from Arabidopsis thaliana (see, Klee et al., Mol. Gen. Genet. 210: pages 437 to 442, 1987) or EPSPS CTP (CTP4) from Petunia hybrida (see, Della-Cioppa et al., Proc. Natl. Acad. Sci. USA 83: pages 6,873 to 6,877, 1986) directs protein sequences of heterologous EPSPS to chloroplasts in transgenic plants (see, U.S. Pat. Nos. 5,627,061; 5,633,435; and 5,312,910; and EP0218571; EP189707; EP508909; and EP924299). To target the chimeric insecticidal proteins to the chloroplast, a sequence encoding a chloroplast transit peptide is placed in operable linkage at the 5′ position of the synthetic coding sequence that encodes the chimeric insecticidal protein designed for expression in plant cells.

The expression cassettes and vectors containing these synthetic or artificial nucleotide sequences were constructed and introduced into plant cells of E. coli and corn (Zea mays), according to the methods and sets of transformation procedures that are known in the art. The transformed corn cells were regenerated into transformed plants expressing the truncated chimeric insecticidal protein. To test the pesticidal activity, bioassays were conducted in the presence of neonate larvae (1 day old) of lepidopteran pests. The compositions of recombinant nucleic acid molecules that encode chimeric insecticidal proteins are contemplated. For example, chimeric insecticidal proteins can be expressed with recombinant DNA constructs, in which a polynucleotide molecule with an ORF encodes the chimeric insecticidal protein operationally linked to gene expression elements such as a promoter and any other regulatory element necessary for expression in the system for which the construct is intended. Non-limiting examples include a functional plant promoter operationally linked to the synthetic chimeric insecticidal protein that encodes sequences for the expression of the chimeric insecticidal protein in plants, or a functional promoter in Bt operationally linked to a chimeric insecticidal protein that encodes the sequence for the expression of the protein in a Bt bacterium or other species of Bacillus. Other elements can be operationally linked to chimeric insecticidal proteins that encode sequences including, without limitation, enhancers, introns, untranslated leaders, encoded protein immobilization peptides (HIS-tag), translocation peptides (i.e., plastid transit peptides, signal peptides), polypeptide sequences for post-translational modification enzymes, ribosomal binding sites, and RNAi target sites.

Examples of recombinant polynucleotide molecules provided herein include, without limitation, a heterologous promoter operationally linked to a polynucleotide such as SEQ ID NOs: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96, which encodes the polypeptide or protein having the amino acid sequence as shown in SEQ ID NOs: 1 (EMS_Q1), 2 (EMS_Q2), 3 (EMS_Q3), 4 (EMS_Q4), 5 (EMS_Q5), 6 (EMS_Q6), 7 (EMS_Q7), 8 (EMS_Q8), 9 (EMS_Q9), 10 (EMS_Q10), 11 (EMS_Q11), 12 (EMS_Q12), 13 (EMS_Q13), 14 (EMS_Q14), 15 (EMS_Q15), 16 (EMS_Q16), 17 (EMS_Q17), 18 (EMS_Q18), 19 (EMS_Q19), 20 (EMS_Q20), 21 (EMS_Q21), 22 (EMS_Q22), 23 (EMS_Q23), 24 (EMS_Q24), 25 (EMS_Q25), 26 (EMS_Q26), 27 (EMS_Q27), 28 (EMS_Q28), 29 (EMS_Q29), 30 (EMS_Q30), 31 (EMS_Q31), 32 (EMS_Q32), 33 (EMS_Q33), 34 (EMS_Q34), 35 (EMS_Q35), 36 (EMS_Q36), 37 (EMS_Q37), 38 (EMS_Q38), 39 (EMS_Q39), 40 (EMS_Q40), 41 (EMS_Q41), 42 (EMS_Q42), 43 (EMS_Q43), 44 (EMS_Q44), 45 (EMS_Q45), 46 (EMS_Q46), 47 (EMS_Q47), and 48 (EMS_Q48). A heterologous promoter can also be operationally linked to coding sequences of synthetic DNA that encode a plastid-targeted truncated chimeric insecticidal protein and a non-targeted truncated chimeric insecticidal protein. It is contemplated that the codons of a molecule of recombinant nucleic acids that encodes a chimeric insecticidal protein disclosed in this document can be replaced by synonymous codons (known in the technique as a silent substitution). Methods of optimizing codons for gene expression in host cells of species of interest, including bacteria and plants, such as dicots and monocots, are known in the prior art. The technician in the subject will know, for example, how to use the Optimizer software (http://genomes.urv.es/OPTIMIZER/Form.php) (Puigbo, P., Guzmen, E., Romeu, A., and Garcia-Vallve, S. 2007 OPTIMIZER: A web server for optimizing the codon usage of DNA sequences. Nucleic Acids Research, 35:W126-W131), selecting parameters for the host cell of interest.

A recombinant DNA molecule, construct, or expression cassette encoding a truncated chimeric insecticidal protein may further comprise a region of DNA encoding one or more toxic agents that can be configured to concomitantly express or co-express a DNA sequence encoding a chimeric insecticidal protein, a protein different from a chimeric insecticidal protein, an insect-inhibiting dsRNA molecule, or a complementary protein. Complementary proteins include, without limitation, cofactors, enzymes, binding partners, or other agents that function to aid in the efficacy of an insect inhibitor agent, for example, to assist in its expression, influence its stability in plants, optimize free energy for oligomerization, increase its toxicity, and expand its spectrum of activity. A complementary protein can facilitate the absorption of one or more insect inhibitors, for example, or enhance the toxic effects of the toxic agent.

A recombinant DNA molecule, construct, or expression cassette can be assembled so that all proteins or dsRNA molecules are expressed from the same promoter, or each protein or dsRNA molecule is under the control of a separate promoter, or some combination of them. The proteins of this invention can be expressed from a multi-gene expression system in which a chimeric insecticidal protein is expressed from a common nucleotide segment, which also contains other open reading frames and promoters, depending on the type of expression system selected. In another example, a plant multigene expression system can use multiple-unlinked expression cassettes, each expressing a different protein or another toxic agent, such as one or more dsRNA molecules.

Recombinant nucleic acid molecules or recombinant DNA constructs comprising a sequence encoding chimeric insecticidal protein can be administered to host cells by vectors, for example, plasmid, baculovirus, synthetic chromosome, virion, cosmid, phagemid, phage, or viral vector. Such vectors can be used to achieve stable or transient expression of a chimeric insecticidal protein-coding sequence in a host cell, or subsequent expression of the encoded polypeptide. An exogenous recombinant polynucleotide or recombinant DNA construct comprising a chimeric insecticidal protein sequence coding sequence and which is introduced into a host cell is also referred to in this document as a “transgene”.

Transgenic bacteria, transgenic plant cells, transgenic plants, and parts of transgenic plants that contain a polynucleotide encoding any one or more of the chimeric insecticidal proteins are provided in this document. The term “bacterial cell” or “bacteria” may include, without limitation, a cell of Agrobacterium, Bacillus, Escherichia, Salmonella, Pseudomonas, or Rhizobium. The term “plant cell” or “plant” may include, without limitation, a dicotyledonous cell or a monocotyledonous cell. Plants and plant cells contemplated include, without limitation, chard, watercress, lettuce, alfalfa, cotton, chicory, alstroemeria, peanuts, rice, oats, potatoes, snapdragon, brachiaria, broccoli, coffee, sugarcane, grass, carrot, rye, barley, chicory, coconut, kale, cauliflower, chrysanthemum, peace lily, spinach, stevia, beans, tobacco, gerbera, gypsophila, lisianthus, castor bean, cassava, passion fruit, millet, corn, mustard, pastures, pepper, bell pepper, cabbage, rose, arugula, rubber tree, soybeans, sorghum, tomato, wheat, triticale, fruits, and vegetables. In certain embodiments, transgenic plants and transgenic plant parts regenerated from a transgenic plant cell are provided. In certain embodiments, transgenic plants can be obtained from a transgenic seed or seedling of a transgenic plant. In certain embodiments, the part of the plant may be a seed, a capsule, a leaf, a flower, a stem, a root, or any portion thereof, or a non-regenerable portion of a part of the transgenic plant. In the context of this document, a “non-regenerable” portion of a transgenic plant part is a portion that cannot be induced to form a whole plant or that cannot be induced to form a whole plant capable of sexual or asexual reproduction. In certain embodiments, a non-regenerable portion of a plant part is a portion of a seed, capsule, leaf, flower, stem, or transgenic root.

This document also provides methods for the production of transgenic plants that comprise lepidopteran inhibitory amounts of truncated chimeric insecticidal proteins. Such plants can be produced by introducing a polynucleotide that encodes the chimeric insecticidal proteins provided in this application into a plant cell, and selecting a plant derived from the said plant cell that expresses an insect- or lepidopteran-inhibiting amount of the chimeric insecticidal protein. Plants can be derived from plant cells by regeneration, seeds, pollen, or meristem transformation techniques. The methods for the transformation of plants are known in the art. For example, Agrobacterium-mediated transformation is described, for example, in documents U.S. Pat. No. 8,404,930 (corn), US 2009/0142837 (corn), WO2011095460, US 2009/0138985 (soybean), US 2008/0280361 (soybean), WO2000071733 (cotton), and US 2008/0256667 (cotton).

Plants expressing chimeric insecticidal proteins can be cross-bred through reproduction with transgenic events expressing other insecticidal proteins and/or expressing other transgenic traits such as other insect control traits, herbicide tolerance genes, genes that confer yield or stress tolerance traits, and the like, or such traits can be combined into a single vector so that the traits are all linked.

Processed plant products, in which the processed product comprises a detectable amount of a truncated chimeric insecticidal protein, are also disclosed in this application. In certain embodiments, the processed product is selected from the group consisting of plant parts, plant biomass, oil, flour, sugar, animal feed, bran, flakes, husks, processed seeds, and seeds. In certain embodiments, the processed product is non-regenerable. The plant product may comprise commodities or other consumer goods products derived from a transgenic plant or a part of a transgenic plant, in which the consumer goods product or other products can be traced through trade by detecting nucleotide or RNA segments or expressed proteins that encode or comprise distinctive portions of a chimeric insecticidal protein.

The methods of controlling insects, in particular, infestations of lepidopteran crops, with chimeric insecticidal proteins are also disclosed in this application. Such methods can comprise the cultivation of a plant comprising an insect-inhibiting amount of the chimeric insecticidal protein. In certain embodiments, such methods may further comprise any one or more of: (i) applying any composition comprising or encoding a chimeric insecticidal protein to a plant or a seed that gives rise to a plant; and (ii) transforming a plant or a plant cell that gives rise to a plant with a polynucleotide that encodes a chimeric insecticidal protein. In general, it is contemplated that the chimeric insecticidal protein can be provided in a composition, provided in a microorganism, or provided in a transgenic plant to confer insect inhibitory activity against lepidopteran insects.

In certain embodiments, the chimeric insecticidal protein is the active ingredient as an insecticide in an insect inhibitor composition prepared by culturing recombinant Bacillus or any other recombinant bacterial cell transformed to express a truncated chimeric insecticidal protein under conditions suitable for expression. Such a composition can be prepared by desiccation, freeze-drying, homogenization, extraction, filtration, centrifugation, sedimentation, or concentration of a culture of such recombinant cells that express/produce the chimeric insecticidal protein. Such a process may result in a Bacillus cell extract or another entomopathogenic bacterial cell extract, cell suspension, cell homogenate, cell lysate, cell supernatant, cell filtrate, or cell sediment. By obtaining the chimeric insecticidal protein as produced, a composition that includes the chimeric insecticidal protein can include bacterial cells, bacterial spores, and parasporal inclusion bodies and can be formulated for various uses, including as agricultural insect inhibitor spray products or as insect inhibitor formulations in dietary bioassays.

The composition or formulation mentioned above may also comprise a vehicle acceptable in agriculture, such as a bait, powder, dust, pellet, granule, spray, emulsion, a colloidal suspension, an aqueous solution, a spore or crystal preparation of Bacillus or a seed treatment. The composition or formulation may also include a recombinant plant cell, plant tissue, seeds, or a plant transformed to express one or more of the proteins; or bacteria transformed to express one or more of the proteins. Depending on the level of insecticidal inhibition or insect inhibition inherent in the recombinant polypeptide and the level of compound or formulation to be applied to a plant or diet assay, the composition or formulation may include various amounts by weight of the recombinant polypeptide, for example, from 0.0001% to 0.001% to 0.01% to 1% to 99% by weight of the recombinant polypeptide.

In one embodiment, in order to reduce the likelihood of resistance development, an insect or transgenic plant inhibitory composition comprising a chimeric insecticidal protein may further comprise at least one additional toxic agent that exhibits insect inhibitory activity against the same Lepidopteran insect species, but which is different from the chimeric insecticidal protein. Additional possible toxic agents for such a composition include an insect-inhibiting protein and an insect-inhibiting dsRNA molecule. An example for the use of such ribonucleotide sequences to control insect pests is described in US 2006/0021087. Such additional polypeptide(s) for the control of lepidopteran pests can be selected from the group consisting of an insect inhibitor protein, such as, but not limited to, Cry1A (U.S. Pat. No. 5,880,275), Cry1Ab, Cry1Ac, Cry1A.105, Cry1Ae, Cry1B (U.S. Ser. No. 10/525,318), Cry1C (U.S. Pat. No. 6,033,874), chimeras Cry1D, Cry1E, Cry1F, and Cry1A/F (U.S. Pat. Nos. 7,070,982; 6,962,705; and 6,713,063), Cry1G, Cry1H, Cry11, Cry1J, Cry1K, Cry1L, Cry2A, Cry2Ab (U.S. Pat. No. 7,064,249), Cry2Ae, Cry4B, Cry6, Cry7, Cry8, Cry9, Cry15, Cry43A, Cry43B, Cry51Aa1, ET66, TIC400, TIC800, TIC834, TIC1415, Vip3A, VIP3Ab, VIP3B, AXMI-001, AXMI-002, AXMI-030, AXMI-035, and AXMI-045 (US 2013/0117884), AXMI-52, AXMI-58, AXMI-88, AXMI-97, AXMI-102, AXMI-112, AXMI-117, AXMI-100 (US 2013/0310543), AXMI-115, AXMI-113, AXMI-005 (US 2013/0104259), AXMI-134 (US 2013/0167264), AXMI-150 (US2010/0160231), AXMI-184 (US 2010/0004176), AXMI-196, AXMI-204, AXMI-207, AXMI-209 (US 2011/0030096), AXMI-218, AXMI-220 (US 2014/0245491), AXMI-221z, AXMI-222z, AXMI-223z, AXMI-224z, AXMI-225z (US 2014/0196175), AXMI-238 (US 2014/0033363), AXMI-270 (US 2014/0223598), AXMI-345 (US 2014/0373195), DIG-3 (US 2013/0219570), DIG-5 (US 2010/0317569), DIG-11 (US 2010/0319093), AflP-1A and derivatives thereof (US 2014/0033361), AflP-1B and derivatives thereof (US 2014/0033361), PIP-1APIP-11B (US 2014/0007292), PSEEN3174 (US 2014/0007292), AECFG-592740 (US 2014/0007292), Pput_1063 (US 2014/0007292), Pput_1064 (US 2014/0007292), GS-135 and derivatives thereof (US 2012/0233726), GS153 and derivatives thereof (US 2012/0192310), GS154 and derivatives thereof (US 2012/0192310), GS155 and derivatives thereof (US 2012/0192310), SEQ ID NO: 2 and derivatives thereof as described in US 2012/0167259, SEQ ID NO:2 and derivatives thereof as described in US 2012/0047606, SEQ ID NO: 2 and derivatives thereof as described in US 2011/0154536, SEQ ID NO:2 and derivatives thereof as described in US 2011/0112013, SEQ ID NO: 2 and 4 and derivatives thereof as described in US 2010/0192256, SEQ ID NO: 2 and derivatives thereof as described in US 2010/0077507, SEQ ID NO: 2 and derivatives thereof as described in US 2010/0077508, SEQ ID NO: 2 and derivatives thereof as described in US 2009/0313721, SEQ ID NO: 2 or 4 and derivatives thereof as described in US 2010/0269221, SEQ ID NO:2 and derivatives thereof as described in U.S. Pat. No. 7,772,465, CF161_0085 and derivatives thereof as described in document WO2014/008054, toxic Lepidoptera proteins and derivatives thereof as described in US 2008/0172762, US 2011/0055968, and US 2012/0117690; SEQ ID NO: 2 and derivatives thereof as described in U.S. Pat. No. 7,510,878, SEQ ID NO: 2 and derivatives thereof as described in U.S. Pat. No. 7,812,129; TIC1100, TIC860, TIC867, TIC868, TIC869, TIC836, TIC713, TIC843, TIC862, TIC1099, TIC1103, TIC845, TIC846, TIC858, TIC866, TIC838, TIC841, TIC842, TIC850, TIC859, TIC861, TIC848, TIC849, and TIC847 as described in document WO2016/061391 and similar.

In other embodiments, an insect inhibitor composition or a transgenic plant may still comprise at least one additional toxic agent that exhibits insect inhibitory activity against an insect pest that is not inhibited by the chimeric insecticidal proteins of the present invention (such as coleopteran, hemipteran, and homopteran pests), in order to expand the obtained spectrum of insect inhibition.

Such an additional toxic agent for the control of coleopteran pests can be selected from the group consisting of an insect inhibitor protein, such as, without limitation, Cry3Bb (U.S. Pat. No. 6,501,009), Cry1C variants, Cry3A variants, Cry3, Cry3B, Cry34/35, 5307, AXMI-134 (US 2013/0167264), AXMI-184 (US 2010/0004176), AXMI-205 (US 2014/0298538), AXMI207 (US 2013/0303440), AXMI-218, AXMI-220 (US 2014/0245491), AXMI-221z, AXMI-223z (US 2014-0196175), AXMI-279 (US 2014/0223599), AXMI-R1 and variants thereof (US 2010/0197592, TIC407, TIC417, TIC431, TIC807, TIC853, TIC901, TIC1201, TIC3131, DIG-10 (US 2010/0319092), eHIPs (US 2010/0017914), IP3 and variants thereof (US 2012/0210462), and Q-Hexatoxin-Hv1a (US 2014/0366227).

Such an additional toxic agent for the control of hemipteran pests can be selected from the group consisting of hemiptera-active proteins, such as, without limitation, TIC1415 (US 2013/0097735), TIC807 (U.S. Pat. No. 8,609,936), TIC834 (US 2013/0269060), AXMI-036 (US 2010/0137216) and AXMI-171 (US 2013/0055469). Additional polypeptides for the control of coleopteran, lepidopteran, and hemipteran insects can be found on the toxin nomenclature web pages of Bacillus thuringiensis (http://www.lifesci.sussex.ac.uk/home/Neil_Crickmore/Bt/ and https://www.bpprc.org/).

Chimeric insecticidal protein coding sequences and sequences that have a substantial percentage of identity with chimeric insecticidal proteins can be identified using methods known to those skilled in the art, such as polymerase chain reaction (PCR), thermal amplification, and hybridization. For example, chimeric insecticidal proteins can be used to produce antibodies that bind specifically to related proteins, and they can be used to search for and find other proteins that are closely related.

In addition, the nucleotide sequences encoding chimeric insecticidal proteins can be used as probes and primers for screening to identify other members of the class using hybridization and isothermal amplification or thermal cycling methods. For example, oligonucleotides derived from sequences of any of SEQ ID NO can be used: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96 to determine the presence or absence of a chimeric insecticidal transgene in a sample of deoxyribonucleic acid derived from a consumer goods product. Given the sensitivity of certain nucleic acid detection methods employing oligonucleotides, it is anticipated that oligonucleotides derived from sequences as presented in any of the SEQ ID NO: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96 can be used to detect a nucleic acid encoding a chimeric insecticidal protein truncated in plant products from a transgenic plant.

### EXAMPLES

Considering the above, the technician in the subject will understand that the following embodiments described are merely representative of the invention, which can be implemented in numerous ways. Accordingly, specific structural and functional details disclosed in this document should not be construed as limiting.

### Example 1

### Creation of Coding Sequences for Truncated Chimeric Insecticidal Proteins Active Against Lepidoptera

This example illustrates the creation of the truncated chimeric insecticidal proteins.

The recombinant nucleic acid sequences were constructed from Cry genes obtained from the sequencing of Bacillus thuringiensis strains. Initially, the C-terminal protoxin domains of each Cry protein were removed, generating the truncated parental Cry proteins Cry1C (SEQ ID NO: 97), Cry1B (SEQ ID NO: 101), Cry1Ab (SEQ ID NO: 105), Cry1E (SEQ ID NO: 110), Cry1 Da (SEQ ID NO: 114), Cry1Fb (SEQ ID NO: 118) and Cry2Aa (SEQ ID NO: 122). Next, domains I, II, and III were identified through alignments of protein sequences with protein domain databases (Pfam or NCBI-BlastP). Table 1 details the identifications of each truncated Cry protein and their respective domains. The truncated chimeric proteins were assembled through the combination of domains as illustrated in Table 2. The resulting polynucleotide sequences were synthesized with codon optimization for expression in Escherichia coli and cloned into a plasmid expression vector and transformed into E. coli. Preparations of proteins expressed in E. coli were used in bioassays against various lepidopteran pest caterpillars to verify insecticidal activity and growth inhibition.

TABLE 1
TRUNCATED CHIMERIC PESTICIDE PROTEINS
AND THEIR COMPONENTS.

| Cry Protein | SEQ ID NO |
|---|---|
| Cry1C_T (Parental) | SEQ ID NO: 97 |
| Cry1C_T_Domain I | SEQ ID NO: 98 |
| Cry1C_T_Domain II | SEQ ID NO: 99 |
| Cry1C_T_Domain III | SEQ ID NO: 100 |
| Cry1B_T (Parental) | SEQ ID NO: 101 |
| Cry1B_T_Domain I | SEQ ID NO: 102 |
| Cry1B_T_Domain II | SEQ ID NO: 103 |
| Cry1B_T_Domain III | SEQ ID NO: 104 |
| Cry1Ab_T (Parental) | SEQ ID NO: 105 |
| Cry1Ab_T_Domain I | SEQ ID NO: 106 |
| Cry1Ab_T_Domain II | SEQ ID NO: 107 |
| Cry1Ab_T_Domain III_a* | SEQ ID NO: 108 |
| Cry1Ab_T_Domain III_b* | SEQ ID NO: 109 |
| Cry1E_T (Parental) | SEQ ID NO: 110 |
| Cry1E_T_Domain I | SEQ ID NO: 111 |
| Cry1E_T_Domain II | SEQ ID NO: 112 |
| Cry1E_T_Domain III | SEQ ID NO: 113 |
| Cry1Da_T (Parental) | SEQ ID NO: 114 |
| Cry1Da_T_Domain I | SEQ ID NO: 115 |
| Cry1Da_T_Domain II | SEQ ID NO: 116 |
| Cry1Da_T_Domain III | SEQ ID NO: 117 |
| Cry1Fb_T (Parental) | SEQ ID NO: 118 |
| Cry1Fb_T_Domain I | SEQ ID NO: 119 |
| Cry1Fb_T_Domain II | SEQ ID NO: 120 |
| Cry1Fb_T_Domain III | SEQ ID NO: 121 |
| Cry2Aa_T (Parental) | SEQ ID NO: 122 |
| Cry2Aa_T_Domain I | SEQ ID NO: 123 |
| Cry2Aa_T_Domain II | SEQ ID NO: 124 |
| Cry2Aa_T_Domain III | SEQ ID NO: 125 |

TABLE 2
TRUNCATED CHIMERIC PESTICIDE PROTEINS AND THEIR COMPONENTS.

| | SEQ | Domain I - SEQ | Domain II - SEQ | Domain III - SEQ ID | |
|---|---|---|---|---|---|
| | ID | ID NO/Parental | ID NO/Parental | NO/Parental | |
| Protein | NO: | Protein | Protein | Protein | Protoxin |
| EMS_Q1 | 1 | 98/Cry1C | 99/Cry1C | 104/Cry1B | ND |
| EMS_Q2 | 2 | 98/Cry1C | 103/Cry1B | 100/Cry1C | ND |
| EMS_Q3 | 3 | 98/Cry1C | 103/Cry1B | 104/Cry1B | ND |
| EMS_Q4 | 4 | 102/Cry1B | 99/Cry1C | 100/Cry1C | ND |
| EMS_Q5 | 5 | 102/Cry1B | 99/Cry1C | 104/Cry1B | ND |
| EMS_Q6 | 6 | 102/Cry1B | 103/Cry1B | 100/Cry1C | ND |
| EMS_Q7 | 7 | 106/Cry1Ab | 107/Cry1Ab | 104/Cry1B | ND |
| EMS_Q8 | 8 | 106/Cry1Ab | 103/Cry1B | 108/Cry1Ab_a | ND |
| EMS_Q9 | 9 | 106/Cry1Ab | 103/Cry1B | 104/Cry1B | ND |
| EMS_Q10 | 10 | 102/Cry1B | 107/Cry1Ab | 108/Cry1Ab_a | ND |
| EMS_Q11 | 11 | 102/Cry1B | 107/Cry1Ab | 104/Cry1B | ND |
| EMS_Q12 | 12 | 102/Cry1B | 103/Cry1B | 108/Cry1Ab_a | ND |
| EMS_Q13 | 13 | 106/Cry1Ab | 107/Cry1Ab | 113/Cry1E | ND |
| EMS_Q14 | 14 | 111/Cry1E | 112/Cry1E | 109/Cry1Ab_b | ND |
| EMS_Q15 | 15 | 111/Cry1E | 107/Cry1Ab | 113/Cry1E | ND |
| EMS_Q16 | 16 | 111/Cry1E | 107/Cry1Ab | 109/Cry1Ab_b | ND |
| EMS_Q17 | 17 | 106/Cry1Ab | 112/Cry1E | 113/Cry1E | ND |
| EMS_Q18 | 18 | 106/Cry1Ab | 112/Cry1E | 109/Cry1Ab_b | ND |
| EMS_Q19 | 19 | 111/Cry1E | 112/Cry1E | 104/Cry1B | ND |
| EMS_Q20 | 20 | 111/Cry1E | 103/Cry1B | 113/Cry1E | ND |
| EMS_Q21 | 21 | 111/Cry1E | 103/Cry1B | 104/Cry1B | ND |
| EMS_Q22 | 22 | 102/Cry1B | 112/Cry1E | 113/Cry1E | ND |
| EMS_Q23 | 23 | 102/Cry1B | 112/Cry1E | 104/Cry1B | ND |
| EMS_Q24 | 24 | 102/Cry1B | 103/Cry1B | 113/Cry1E | ND |
| EMS_Q25 | 25 | 98/Cry1C | 99/Cry1C | 109/Cry1Ab_b | ND |
| EMS_Q26 | 26 | 98/Cry1C | 107/Cry1Ab | 100/Cry1C | ND |
| EMS_Q27 | 27 | 98/Cry1C | 107/Cry1Ab | 109/Cry1Ab_b | ND |
| EMS_Q28 | 28 | 106/Cry1Ab | 99/Cry1C | 100/Cry1C | ND |
| EMS_Q29 | 29 | 106/Cry1Ab | 99/Cry1C | 109/Cry1Ab_b | ND |
| EMS_Q30 | 30 | 106/Cry1Ab | 107/Cry1Ab | 100/Cry1C | ND |
| EMS_Q31 | 31 | 119/Cry1Fb | 116/Cry1Da | 117/Cry1Da | ND |
| EMS_Q32 | 32 | 115/Cry1Da | 116/Cry1Da | 121/Cry1Fb | ND |
| EMS_Q33 | 33 | 115/Cry1Da | 120/Cry1Fb | 117/Cry1Da | ND |
| EMS_Q34 | 34 | 115/Cry1Da | 120/Cry1Fb | 121/Cry1Fb | ND |
| EMS_Q35 | 35 | 119/Cry1Fb | 116/Cry1Da | 121/Cry1Fb | ND |
| EMS_Q36 | 36 | 119/Cry1Fb | 120/Cry1Fb | 117/Cry1Da | ND |
| EMS_Q37 | 37 | 111/Cry1E | 112/Cry1E | 100/Cry1C | ND |
| EMS_Q38 | 38 | 111/Cry1E | 99/Cry1C | 113/Cry1E | ND |
| EMS_Q39 | 39 | 111/Cry1E | 99/Cry1C | 100/Cry1C | ND |
| EMS_Q40 | 40 | 98/Cry1C | 112/Cry1E | 113/Cry1E | ND |
| EMS_Q41 | 41 | 98/Cry1C | 112/Cry1E | 100/Cry1C | ND |
| EMS_Q42 | 42 | 98/Cry1C | 99/Cry1C | 113/Cry1E | ND |
| EMS_Q43 | 43 | 123/Cry2Aa | 124/Cry2Aa | 100/Cry1C | ND |
| EMS_Q44 | 44 | 123/Cry2Aa | 99/Cry1C | 125/Cry2Aa | ND |
| EMS_Q45 | 45 | 123/Cry2Aa | 99/Cry1C | 100/Cry1C | ND |
| EMS_Q46 | 46 | 98/Cry1C | 124/Cry2Aa | 125/Cry2Aa | ND |
| EMS_Q47 | 47 | 98/Cry1C | 124/Cry2Aa | 100/Cry1C | ND |
| EMS_Q48 | 48 | 98/Cry1C | 99/Cry1C | 125/Cry2Aa | ND |

### Example 2

Test for Activity of Truncated Chimeric Proteins Against S. frugiperda

This example illustrates the testing of the truncated chimeric proteins described in Example 1 for insecticidal activity against S. frugiperda.

The polynucleotide sequences encoding the truncated chimeric proteins were expressed in E. coli and used in bioassays with neonate caterpillars of S. frugiperda. The activity was determined from the evaluation of mortality scores or larval growth inhibition during the period of 5 to 7 days of feeding on an artificial diet containing the recombinant chimeric protein preparation. Table 3 illustrates the results of insecticidal activity of each chimeric protein against S. frugiperda. Insecticidal activity is represented by the “+” signs. The “−” sign indicates that no insecticidal activity has been observed and NT means that the protein has not been tested. Distilled water and E. coli containing the empty expression vector were used as negative controls. The mortality of the controls was below 15%.

TABLE 3
INSECTICIDAL ACTIVITY (MORTALITY OR
GROWTH INHIBITION) OF TRUNCATED CHIMERIC
PROTEINS AGAINST S. FRUGIPERDA FROM
EXPRESSION IN E. COLI.

| SEQ ID NO of the | Protein | Activity against |
|---|---|---|
| protein | ID | S. frugiperda |
| SEQ ID NO: 1 | EMS_Q1 | + |
| SEQ ID NO: 2 | EMS_Q2 | + |
| SEQ ID NO: 3 | EMS_Q3 | + |
| SEQ ID NO: 4 | EMS_Q4 | + |
| SEQ ID NO: 5 | EMS_Q5 | + |
| SEQ ID NO: 6 | EMS_Q6 | + |
| SEQ ID NO: 7 | EMS_Q7 | − |
| SEQ ID NO: 8 | EMS_Q8 | + |
| SEQ ID NO: 9 | EMS_Q9 | − |
| SEQ ID NO: 10 | EMS_Q10 | + |
| SEQ ID NO: 11 | EMS_Q11 | − |
| SEQ ID NO: 12 | EMS_Q12 | + |
| SEQ ID NO: 13 | EMS_Q13 | − |
| SEQ ID NO: 14 | EMS_Q14 | − |
| SEQ ID NO: 15 | EMS_Q15 | + |
| SEQ ID NO: 16 | EMS_Q16 | − |
| SEQ ID NO: 17 | EMS_Q17 | − |
| SEQ ID NO: 18 | EMS_Q18 | − |
| SEQ ID NO: 19 | EMS_Q19 | + |
| SEQ ID NO: 20 | EMS_Q20 | + |
| SEQ ID NO: 21 | EMS_Q21 | + |
| SEQ ID NO: 22 | EMS_Q22 | + |
| SEQ ID NO: 23 | EMS_Q23 | − |
| SEQ ID NO: 24 | EMS_Q24 | + |
| SEQ ID NO: 25 | EMS_Q25 | + |
| SEQ ID NO: 26 | EMS_Q26 | + |
| SEQ ID NO: 27 | EMS_Q27 | − |
| SEQ ID NO: 28 | EMS_Q28 | + |
| SEQ ID NO: 29 | EMS_Q29 | − |
| SEQ ID NO: 30 | EMS_Q30 | − |
| SEQ ID NO: 31 | EMS_Q31 | − |
| SEQ ID NO: 32 | EMS_Q32 | + |
| SEQ ID NO: 33 | EMS_Q33 | + |
| SEQ ID NO: 34 | EMS_Q34 | NT |
| SEQ ID NO: 35 | EMS_Q35 | + |
| SEQ ID NO: 36 | EMS_Q36 | − |
| SEQ ID NO: 37 | EMS_Q37 | + |
| SEQ ID NO: 38 | EMS_Q38 | − |
| SEQ ID NO: 39 | EMS_Q39 | − |
| SEQ ID NO: 40 | EMS_Q40 | − |
| SEQ ID NO: 41 | EMS_Q41 | − |
| SEQ ID NO: 42 | EMS_Q42 | + |
| SEQ ID NO: 43 | EMS_Q43 | + |
| SEQ ID NO: 44 | EMS_Q44 | NT |
| SEQ ID NO: 45 | EMS_Q45 | NT |
| SEQ ID NO: 46 | EMS_Q46 | − |
| SEQ ID NO: 47 | EMS_Q47 | − |
| SEQ ID NO: 48 | EMS_Q48 | + |

### Example 3

### Synthesis of Genes Encoding Truncated Chimeric Insecticidal Proteins for Expression in Plants

This example illustrates the synthesis of polynucleotides that encode chimeric insecticidal proteins for expression in plants.

The modification of gene codons was done with the aid of the Optimizer (http://genomes.urv.es/OPTIMIZER/Form.php) software (Puigbo P., Guzmen E., Romeu A., and Garcia-Vallve S. 2007 OPTIMIZER: A web server for optimizing the codon usage of DNA sequences. Nucleic Acids Research, 35:W126-W131), targeting codons more compatible with corn (Zea mays), and the sequences were sent digitally for commercial synthesis. The optimized gene was synthesized in the pBS plasmid and cloned in the binary vector p7i2x-UibZm (https://dna-cloning.com/binaries/). The synthesized sequences were confirmed by sequencing, according to standard techniques.

TABLE 4
POLYNUCLEOTIDE SEQUENCES ENCODING THE NOVEL
CHIMERIC PROTEINS FOR EXPRESSION IN PLANTS.

| Protein | SEQ ID NO: of | SEQ ID |
|---|---|---|
| ID | the chimera | NO: of DNA |
| EMS_Q1 | SEQ ID NO: 1 | SEQ ID NO: 49 |
| EMS_Q2 | SEQ ID NO: 2 | SEQ ID NO: 50 |
| EMS_Q3 | SEQ ID NO: 3 | SEQ ID NO: 51 |
| EMS_Q4 | SEQ ID NO: 4 | SEQ ID NO: 52 |
| EMS_Q5 | SEQ ID NO: 5 | SEQ ID NO: 53 |
| EMS_Q6 | SEQ ID NO: 6 | SEQ ID NO: 54 |
| EMS_Q7 | SEQ ID NO: 7 | SEQ ID NO: 55 |
| EMS_Q8 | SEQ ID NO: 8 | SEQ ID NO: 56 |
| EMS_Q9 | SEQ ID NO: 9 | SEQ ID NO: 57 |
| EMS_Q10 | SEQ ID NO: 10 | SEQ ID NO: 58 |
| EMS_Q11 | SEQ ID NO: 11 | SEQ ID NO: 59 |
| EMS_Q12 | SEQ ID NO: 12 | SEQ ID NO: 60 |
| EMS_Q13 | SEQ ID NO: 13 | SEQ ID NO: 61 |
| EMS_Q14 | SEQ ID NO: 14 | SEQ ID NO: 62 |
| EMS_Q15 | SEQ ID NO: 15 | SEQ ID NO: 63 |
| EMS_Q16 | SEQ ID NO: 16 | SEQ ID NO: 64 |
| EMS_Q17 | SEQ ID NO: 17 | SEQ ID NO: 65 |
| EMS_Q18 | SEQ ID NO: 18 | SEQ ID NO: 66 |
| EMS_Q19 | SEQ ID NO: 19 | SEQ ID NO: 67 |
| EMS_Q20 | SEQ ID NO: 20 | SEQ ID NO: 68 |
| EMS_Q21 | SEQ ID NO: 21 | SEQ ID NO: 69 |
| EMS_Q22 | SEQ ID NO: 22 | SEQ ID NO: 70 |
| EMS_Q23 | SEQ ID NO: 23 | SEQ ID NO: 71 |
| EMS_Q24 | SEQ ID NO: 24 | SEQ ID NO: 72 |
| EMS_Q25 | SEQ ID NO: 25 | SEQ ID NO: 73 |
| EMS_Q26 | SEQ ID NO: 26 | SEQ ID NO: 74 |
| EMS_Q27 | SEQ ID NO: 27 | SEQ ID NO: 75 |
| EMS_Q28 | SEQ ID NO: 28 | SEQ ID NO: 76 |
| EMS_Q29 | SEQ ID NO: 29 | SEQ ID NO: 77 |
| EMS_Q30 | SEQ ID NO: 30 | SEQ ID NO: 78 |
| EMS_Q31 | SEQ ID NO: 31 | SEQ ID NO: 79 |
| EMS_Q32 | SEQ ID NO: 32 | SEQ ID NO: 80 |
| EMS_Q33 | SEQ ID NO: 33 | SEQ ID NO: 81 |
| EMS_Q34 | SEQ ID NO: 34 | SEQ ID NO: 82 |
| EMS_Q35 | SEQ ID NO: 35 | SEQ ID NO: 83 |
| EMS_Q36 | SEQ ID NO: 36 | SEQ ID NO: 84 |
| EMS_Q37 | SEQ ID NO: 37 | SEQ ID NO: 85 |
| EMS_Q38 | SEQ ID NO: 38 | SEQ ID NO: 86 |
| EMS_Q39 | SEQ ID NO: 39 | SEQ ID NO: 87 |
| EMS_Q40 | SEQ ID NO: 40 | SEQ ID NO: 88 |
| EMS_Q41 | SEQ ID NO: 41 | SEQ ID NO: 89 |
| EMS_Q42 | SEQ ID NO: 42 | SEQ ID NO: 90 |
| EMS_Q43 | SEQ ID NO: 43 | SEQ ID NO: 91 |
| EMS_Q44 | SEQ ID NO: 44 | SEQ ID NO: 92 |
| EMS_Q45 | SEQ ID NO: 45 | SEQ ID NO: 93 |
| EMS_Q46 | SEQ ID NO: 46 | SEQ ID NO: 94 |
| EMS_Q47 | SEQ ID NO: 47 | SEQ ID NO: 95 |
| EMS_Q48 | SEQ ID NO: 48 | SEQ ID NO: 96 |

### Example 4

### Expression Cassettes for the Expression of Chimeric Insecticidal Proteins in Plants

This example illustrates the construction of expression cassettes comprising polynucleotide sequences for use in plants that encode truncated chimeric insecticidal proteins.

A variety of plant expression cassettes was constructed with the polynucleotide sequences listed in Table 4. The optimized genes were inserted into the binary vector p7i2x-UibZm (https://dna-cloning.com/binaries/) containing the promoter of the corn ubiquitin gene (ubi) and the terminator of the nopaline synthase gene (nos), generating a series of plasmids designed to allow the protein to be translated and remain in the cytosol of the plant. The resulting plasmids containing the genes encoding the chimeric proteins were used for the transformation of Agrobacterium tumefaciens EHA101 bacteria via electroporation (BioRad/MicroPulser). The transformed bacteria, containing the expression cassettes, were used in the genetic transformation of corn.

### Example 5

### Lepidopteran Activity of Chimeric Insecticidal Proteins in Stably Transformed Maize

This example illustrates the test of the new chimeric proteins described in Example 1, expressed in corn, for insecticidal activity against lepidopteran pests in plants.

From the transformation vectors constructed and described in Example 4, the one containing the polynucleotide sequence SEQ ID NO: was selected. 54 for transformation of the Hill corn variety (Armstrong C L, Grenn C E, Phillips R L (1991). Development and availability of germplasm with high type II culture formation response. Maize Genet. Coop. Newsletter. 65:92-93). The transformation protocol was the one described by Frame et al. Agrobacterium tumefaciens-mediated transformation of corn embryos using a standard binary vector system. Plant Physiol. 2002 May; 129(1):13-22, with minor modifications. Briefly, for the transformation of this genotype, immature embryos between 1.8-2.0 mm in length (10-12 days after pollination) were collected. Ears used for embryo collection were dipped in a 1:1 solution of commercial bleach (2.5% sodium hypochlorite) and distilled water with 1-2 drops of Tween 20 for 20 minutes. They were then rinsed with sterile distilled water for 5 minutes, twice.

The immature embryos were collected with the aid of a spatula from a superficial cut of the grains. For the transfer of the gene construct to corn, Agrobacterium tumefaciens EHA101 was used. From a stock culture of A. tumefaciens containing the gene construct of interest, maintained in glycerol at −80° C., a streak was made on YEP medium (5 g·L⁻¹ yeast extract; 10 g·L⁻¹ peptone; 5 g·L⁻¹ NaCl; 15 g·L⁻¹ bacto agar) containing the necessary antibiotics (spectinomycin 100 mg·L-1 and kanamycin 50 mg·L⁻¹) and the plate was incubated for 2 to 3 days at 28° C. (master plate).

For the genetic transformation, a streak of Agrobacterium was made in YEP medium containing the necessary antibiotics, using a colony isolated from the master plate. The plate was incubated for 2 to 5 days at 19° C. Next, the Agrobacterium was resuspended in infection medium (4.0 g·L⁻¹ N6 salts; 68.4 g·L⁻¹ sucrose; 36.0 g·L⁻¹ glucose; 0.7 g·L⁻¹ proline; 1.5 mg·L-1 2,4-D; 1.0 mL·L⁻¹ N6 vitamins (1000×=1.0 g·L⁻¹ thiamine HCl; 0.5 g·L⁻¹ pyridoxine HCl; 0.5 g·L⁻¹ nicotinic acid); pH 5.2) supplemented with 100 μM acetosyringone to reach an OD550=0.3-0.4 and incubated in a shaker at about 150 rpm, 23° C. for 2 hours.

For the infection of immature corn embryos, 50 to 100 embryos were collected in 1 mL of infection medium supplemented with acetosyringone. After collection, the embryos were rinsed twice, 1 mL of bacterial culture was added, and the suspension was incubated for five minutes at 23° C. After infection, the embryos were transferred to the surface of the co-culture medium (4.0 g·L⁻¹ of N6 salts; 1.5 mg·L⁻¹ of 2,4-D; 30.0 g·L⁻¹ of sucrose; 0.7 g·L-1 of proline; 1.0 mL·L⁻¹ of N6 vitamins (1000×); 0.85 mg·L⁻¹ of AgNO3; 100 μM of acetosyringone; 300 mg·L⁻¹ of L-cysteine; 3.0 g·L⁻¹ of phytagel; pH 5.8) with the scutellum facing up. The plates were incubated in the dark at 20° C. for 3 to 5 days. After co-culture, the embryos were transferred to the resting medium (4.0 g·L-1 of N6 salts; 1.5 mg·L⁻¹ of 2,4-D; 30.0 g·L⁻¹ of sucrose; 0.5 g·L⁻¹ of MES; 0.7 g·L⁻¹ of proline; 1.0 mL·L-1 of N6 vitamins (1000×); 0.85 mg·L⁻¹ of AgNO3; 100 mg·L⁻¹ of Tioxin; 3.0 g·L⁻¹ of phytagel; pH 5.8) at 28° C. (dark) for 7 to 15 days. Then, the embryos were transferred to the selection medium (4.0 g·L⁻¹ of N6 salts; 1.5 mg·L⁻¹ of 2,4-D; 30.0 g·L⁻¹ of sucrose; 0.5 g·L⁻¹ of MES; 0.7 g·L-1 of proline; 1.0 mL·L⁻¹ of N6 vitamins (1000×); 0.85 mg·L⁻¹ of AgNO3; 100 mg·L⁻¹ of Tioxin; 1.5 and 3.0 mg/L of bialaphos; 3.0 g·L⁻¹ of phytagel; pH 5.8) (25 embryos/plate). Sub-cultures of these embryos in selective medium are carried out every 15 days until the selection of calluses growing vigorously.

Selected calluses were transferred to regeneration medium (4.62 g·L⁻¹ of MS salts; 60.0 g·L⁻¹ of sucrose; 100 mg·L⁻¹ of myo-inositol; 1.0 mL·L⁻¹ of MS vitamins (1000×); 1.5 mg/L of bialaphos; 4.0 g·L-1 of phytagel; pH 5.8) and incubated at 26±2° C. (dark) for 15 to 21 days. Calluses ready for germination, with a dry appearance and opaque white color, were transferred to the germination medium (4.62 g·L-1 of MS salts; 30.0 g·L⁻¹ sucrose; 100 mg·L⁻¹ myo-inositol; 1.0 mL·L⁻¹ MS vitamins (1000×=0.5 g·L⁻¹ thiamine HCl; 0.5 g·L⁻¹ pyridoxine HCl; 0.05 g·L⁻¹ nicotinic acid); 3.0 g·L⁻¹ phytagel; pH 5.8) (12 calluses per plate), 25° C., 80-100 μE/m2/sec light intensity, 16-hour photoperiod). Seedlings with leaves and roots were transferred to the greenhouse within 14 to 20 days.

When the roots were well developed and the leaf structures measured about 5 cm in length, the seedlings were transplanted into pots in a greenhouse containing a mixture of soil and organic matter (⅔ soil and ⅓ commercially produced organic matter (TDP 30/15)).

Genetically modified corn plants containing the gene encoding the chimeric protein (SEQ ID NO: 6) were used for infestation assays in the field and in the greenhouse, as well as bioassays for feeding caterpillars with detached leaves.

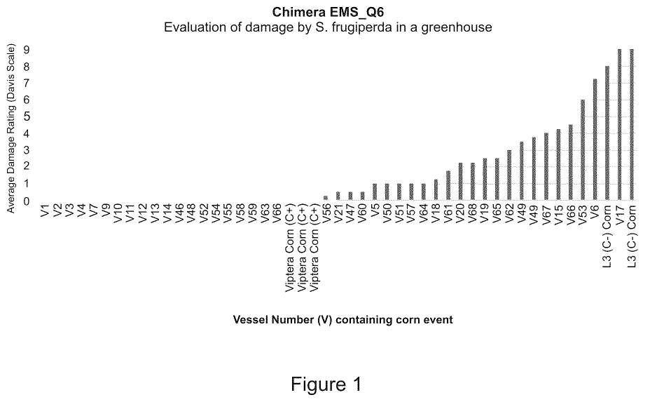

In the greenhouse assay, 44 pots containing corn events with the coding sequence of the chimeric protein EMS_Q6 (SEQ ID NO: 6). Non-transgenic corn L3 was added as a negative control (C−), and a commercial transgenic corn for resistance to S. frugiperda was added as a positive control (C+). The assay was artificially infested with neonate larvae of S. frugiperda at the V4-V6 stage of corn development, and the plants were evaluated by injury scores 13 days after infestation on the Davis scale (Davis, et al.). 1992), where a score of 0 represents no visible damage and a score of 9 represents the plant completely destroyed). FIG. 1 illustrates the result of the injury scores, where a series of events expressing the chimeric protein EMS_Q6 showed an injury score of 0, similar to the vessels of the commercial positive control, while the negative controls showed injury scores ranging from 8 to 9.

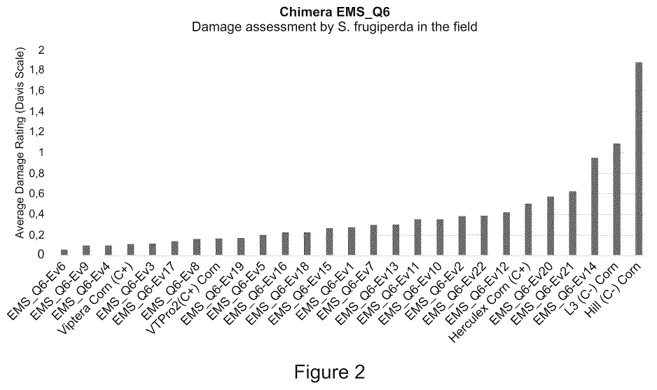

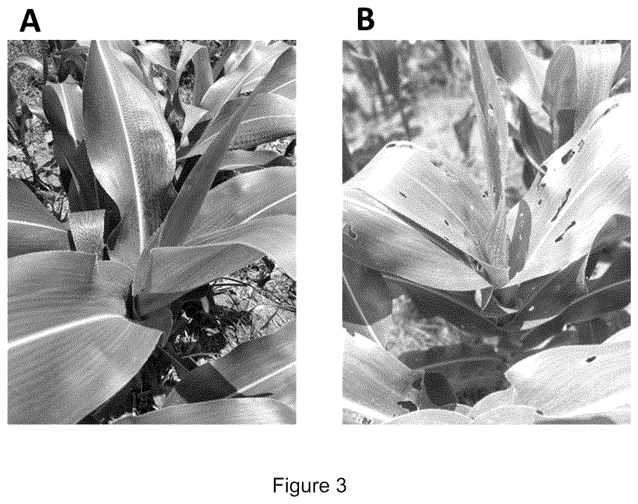

In the field trial, 22 events (Ev1 to Ev22) of corn containing the sequence encoding the chimeric protein EMS_Q6 (SEQ ID NO: 6), two positive controls (commercial transgenic corn resistant to S. frugiperda) and two negative controls (non-transgenic corn). The trial was artificially infested with neonate larvae of S. frugiperda at the V4-V6 stage of corn development, and the plants were evaluated by injury scores at 7, 14, and 21 days after infestation on the Davis scale (Davis, F. M.; NG, S.; Williams, W. P. 1992. Visual rating scales for screening whole-stage corn resistance to fall armyworm. Mississippi: Mississippi State University, Technical Bulletin, v. 186. 9p), where score 0 represents no visible damage and score 9 represents the plant completely destroyed). FIG. 2 illustrates the result of the injury scores represented by the arithmetic mean of the 3 evaluations. It is noted that a large part of the transgenic events containing the protein EMS_06 obtained lower average scores than the negative controls, and some of them received lower scores than the evaluated commercial transgenic products. FIG. 3 illustrates a representative photo of the damage caused by S. frugiperda in a control plant, compared to a transgenic plant expressing the chimeric protein EMS_Q6 (SEQ ID NO: 6). It is noted that the transgenic corn plant does not present damage, while the conventional corn plant presents evident leaf damage caused by the attack of S. frugiperda.

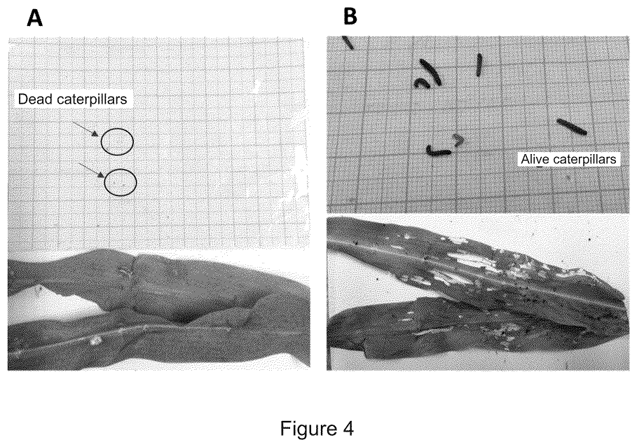

A bioassay was also performed in the laboratory with infestation of leaves detached from one of the corn events containing the coding sequence of the chimeric protein EMS_Q6 (SEQ ID NO: 6) and a non-transgenic corn control. To perform this assay, intact leaves of approximately 15 cm in length were used, from one of the events containing the chimeric protein EMS_Q6 (SEQ ID NO: 6) and a negative control (C−), planted in a greenhouse. The leaves were individually rolled and placed in 50 ml clear plastic cups. On each leaf, 15 neonate caterpillars of S. frugiperda were placed, and the cups were placed in a climate-controlled chamber with a constant temperature of 25° C., 70% humidity, and a photoperiod of 16 hours of light and 8 hours of darkness. After 5 days, the number of live caterpillars present in each cup was evaluated. FIG. 4 illustrates the result of the bioassay. It is noted that in the corn leaf expressing the EMS_Q6 protein, there was total mortality of the caterpillars and absence of leaf damage. In the control corn, there was evident damage from feeding and the presence of live and well-developed caterpillars.

All compositions disclosed and claimed herein may be made and performed without undue experimentation in light of this disclosure. Although the compositions of this invention have been described in terms of the preceding illustrative embodiments, it will be evident to those skilled in the art that variations, changes, modifications, and alterations can be applied to the compositions described in this document without departing from the true concept, essence, and scope of the disclosure. More specifically, it will be apparent that certain agents that are chemically and physiologically related can be substituted for the agents described in this document while the same or similar results would be achieved. All such substitutes and modifications that are apparent to those skilled in the art are considered to be within the essence, scope, and concept of the invention as defined by the accompanying claims.

All publications and patent documents published in the specification are incorporated into this document by reference in their entirety as if each individual publication or patent application were specifically or individually indicated to be incorporated by reference.

1. A truncated chimeric insecticidal protein comprising:
(i) a Domain I of a Cry protein from any of SEQ ID NO: 98, SEQ ID NO: 102, SEQ ID NO: 106, SEQ ID NO: 111, SEQ ID NO: 115, SEQ ID NO: 119 or SEQ ID NO: 123;
(ii) a Domain II of a Cry protein from any of SEQ ID NO: 99, SEQ ID NO: 103, SEQ ID NO: 107, SEQ ID NO: 112, SEQ ID NO: 116, SEQ ID NO: 120 or SEQ ID NO: 124; and
(iii) a Domain III of a Cry protein from any of SEQ ID NO: 100, SEQ ID NO: 104, SEQ ID NO: 108, SEQ ID NO: 109, SEQ ID NO: 113, SEQ ID NO: 117, SEQ ID NO: 121 or SEQ ID NO: 125,
in which the protein lacks a carboxy-terminal protoxin domain.

2. A truncated chimeric insecticidal protein comprising an amino acid sequence that is at least 85% identical to SEQ ID NO: 1; at least 80% identical to SEQ ID NO: 2, at least 79% identical to SEQ ID NO: 3; at least 82% identical to SEQ ID NO: 4; at least 79% identical to SEQ ID NO: 5; at least 87% identical to SEQ ID NO: 6; at least 91% identical to SEQ ID NO: 7; at least 77% identical to SEQ ID NO: 8; at least 81% identical to SEQ ID NO: 9; at least 82% identical to SEQ ID NO: 10; at least 76% identical to SEQ ID NO: 11; at least 91% identical to SEQ ID NO: 12; at least 92% identical to SEQ ID NO: 13; at least 86% identical to SEQ ID NO: 14; at least 82% identical to SEQ ID NO: 15; at least 88% identical to SEQ ID NO: 16; at least 97% identical to SEQ ID NO: 17; at least 81% identical to SEQ ID NO:18; at least 86% identical to SEQ ID NO: 19; at least 76% identical to SEQ ID NO: 20; at least 78% identical to SEQ ID NO: 21; at least 78% identical to SEQ ID NO: 22; at least 78% identical to SEQ ID NO:23; at least 86% identical to SEQ ID NO:24; at least 85% identical to SEQ ID NO: 25; at least 83% identical to SEQ ID NO: 26; at least 83% identical to SEQ ID NO: 27, at least 85% identical to SEQ ID NO: 28, at least 79% identical to SEQ ID NO: 29, at least 85% identical to SEQ ID NO: 30, at least 80% identical to SEQ ID NO: 31, at least 84% identical to SEQ ID NO: 33, at least 78% identical to SEQ ID NO: 33, at least 85% identical to SEQ ID NO: 34, at least 80% identical to SEQ ID NO: 35, at least 87% identical to SEQ ID NO: 36, at least 93% identical to SEQ ID NO: 37, at least 84% identical to SEQ ID NO: 38, at least 81% identical to SEQ ID NO: 39, at least 80% identical to SEQ ID NO: 40, at least 84% identical to SEQ ID NO: 41, at least 93% identical to SEQ ID NO: 42, at least 86% identical to SEQ ID NO: 43, at least 70% identical to SEQ ID NO: 44, at least 75% identical to SEQ ID NO: 45, at least 73% identical to SEQ ID NO: 46, at least 50% identical to SEQ ID NO: 47, or at least 50% identical to SEQ ID NO: 48.

3. A truncated chimeric insecticidal protein, according to claim 1, in which the insecticidal protein has inhibitory activity against an insect species of the order Lepidoptera.

4. A truncated chimeric insecticidal protein, according to claim 1, comprising the amino acid sequence of any of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48.

5. A recombinant nucleic acid molecule, in which the recombinant nucleic acid molecule encodes a truncated chimeric insecticide protein as defined in claim 1.

6. The recombinant nucleic acid molecule according to claim 5, where the recombinant nucleic acid molecule comprises a functional plant promoter operationally linked to the coding sequence of the truncated chimeric insecticide protein.

7. The recombinant nucleic acid molecule according to claim 5, where the recombinant nucleic acid molecule comprises a nucleotide sequence of any of SEQ ID NOs: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96, where the polynucleotide lacks a region coding for a carboxy-terminal protoxin domain.

8. A host cell comprising a nucleic acid molecule of claim 5.

9. An insect inhibitory composition comprising a truncated chimeric insecticidal protein of claim 1.

10. A plant genome comprising a molecule of nucleic acid from claim 5.

11. A plant or plant seed comprising a truncated chimeric insecticidal protein as defined in claim 1.

12. The plant or seed of a plant according to claim 11, where the plant is a plant of chard, watercress, lettuce, alfalfa, cotton, chicory, alstroemeria, peanut, rice, oats, potato, snapdragon, brachiaria, broccoli, coffee, sugarcane, grass, carrot, rye, barley, chicory, coconut, kale, cauliflower, chrysanthemum, peace lily, spinach, stevia, bean, tobacco, gerbera, baby's breath, lisianthus, castor bean, cassava, passion fruit, millet, corn, mustard, pastures, pepper, bell pepper, cabbage, rose, arugula, rubber tree, soybean, sorghum, tomato, wheat, triticale, fruits, and vegetables.

13. A method for controlling a lepidopteran pest comprising placing the lepidopteran pest in contact with an inhibitory amount of a truncated chimeric insecticidal protein of claim 1.

14. A method of controlling a lepidopteran pest comprising exposing the pest to a transgenic plant cell, plant, or plant part, in which the said plant cell, plant, or plant part expresses a recombinant nucleic acid molecule of claim 5.

15. A plant product, characterized by the fact that it comprises a detectable amount of a chimeric insecticidal protein of claim 1.

16. Use of a plant cell, plant, plant part, or seed expressing a truncated chimeric insecticidal protein of claim 1, comprising a recombinant nucleic acid molecule which encodes a truncated chimeric insecticide protein as defined in claim 1, characterized as being for its crossing with a second plant, regeneration of a transgenic plant, planting or growth of a field of transgenic plants, production of plant products or production of a consumer good product.


SEQUENCE LISTING (125 sequences)

SEQ ID NO: 1 - protein, 651 aa, synthetic construct
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSARTNTIGPNRITQIPLVKALNLHSGVTGVRGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSD

SEQ ID NO: 2 - protein, 681 aa, synthetic construct
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGTSAQLTREIYTDPIGRTNAPSGFASTNWFNNNAPSFSAIEAAIIRPPHLLDFPEQLTIYSASSRWSSTQHMIYWVGHRLNFRPIGGTLNTSTQGLTNNTSINPVTLQFTSRDVYRTESYAGTNILFTTPVNGVPWARFNFINPQNIYERGTTTYSQPYQGVGIQLFDSETELPPETTERPNYESYSHRLSHIGLIIGNTLRAPVYSWTHRSADTLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 3 - protein, 668 aa, synthetic construct
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGTSAQLTREIYTDPIGRTNAPSGFASTNWFNNNAPSFSAIEAAIIRPPHLLDFPEQLTIYSASSRWSSTQHMIYWVGHRLNFRPIGGTLNTSTQGLTNNTSINPVTLQFTSRDVYRTESYAGTNILFTTPVNGVPWARFNFINPQNIYERGTTTYSQPYQGVGIQLFDSETELPPETTERPNYESYSHRLSHIGLIIGNTLRAPVYSWTHRSADRTNTIGPNRITQIPLVKALNLHSGVTGVRGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSD

SEQ ID NO: 4 - protein, 686 aa, synthetic construct
MTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNQEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 5 - protein, 673 aa, synthetic construct
MTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNQEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSARTNTIGPNRITQIPLVKALNLHSGVTGVRGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSD

SEQ ID NO: 6 - protein, 703 aa, synthetic construct
MTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNQEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTSAQLTREIYTDPIGRTNAPSGFASTNWFNNNAPSFSAIEAAIIRPPHLLDFPEQLTIYSASSRWSSTQHMIYWVGHRLNFRPIGGTLNTSTQGLTNNTSINPVTLQFTSRDVYRTESYAGTNILFTTPVNGVPWARFNFINPQNIYERGTTTYSQPYQGVGIQLFDSETELPPETTERPNYESYSHRLSHIGLIIGNTLRAPVYSWTHRSADTLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 7 - protein, 657 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFRTNTIGPNRITQIPLVKALNLHSGVTGVRGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSD

SEQ ID NO: 8 - protein, 666 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTSAQLTREIYTDPIGRTNAPSGFASTNWFNNNAPSFSAIEAAIIRPPHLLDFPEQLTIYSASSRWSSTQHMIYWVGHRLNFRPIGGTLNTSTQGLTNNTSINPVTLQFTSRDVYRTESYAGTNILFTTPVNGVPWARFNFINPQNIYERGTTTYSQPYQGVGIQLFDSETELPPETTERPNYESYSHRLSHIGLIIGNTLRAPVYSWTHRSADNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSD

SEQ ID NO: 9 - protein, 666 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTSAQLTREIYTDPIGRTNAPSGFASTNWFNNNAPSFSAIEAAIIRPPHLLDFPEQLTIYSASSRWSSTQHMIYWVGHRLNFRPIGGTLNTSTQGLTNNTSINPVTLQFTSRDVYRTESYAGTNILFTTPVNGVPWARFNFINPQNIYERGTTTYSQPYQGVGIQLFDSETELPPETTERPNYESYSHRLSHIGLIIGNTLRAPVYSWTHRSADRTNTIGPNRITQIPLVKALNLHSGVTGVRGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSD

SEQ ID NO: 10 - protein, 681 aa, synthetic construct
MTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNQEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSD

SEQ ID NO: 11 - protein, 681 aa, synthetic construct
MTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNQEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFRTNTIGPNRITQIPLVKALNLHSGVTGVRGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSD

SEQ ID NO: 12 - protein, 690 aa, synthetic construct
MTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNQEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTSAQLTREIYTDPIGRTNAPSGFASTNWFNNNAPSFSAIEAAIIRPPHLLDFPEQLTIYSASSRWSSTQHMIYWVGHRLNFRPIGGTLNTSTQGLTNNTSINPVTLQFTSRDVYRTESYAGTNILFTTPVNGVPWARFNFINPQNIYERGTTTYSQPYQGVGIQLFDSETELPPETTERPNYESYSHRLSHIGLIIGNTLRAPVYSWTHRSADNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSD

SEQ ID NO: 13 - protein, 657 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFTNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATFEEEYDLERAQKAVNALFTSTNQLGLKTDVTDYHIDQVSNLVECL

SEQ ID NO: 14 - protein, 610 aa, synthetic construct
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIPTSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSANNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLER

SEQ ID NO: 15 - protein, 656 aa, synthetic construct
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIPTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFTNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATFEEEYDLERAQKAVNALFTSTNQLGLKTDVTDYHIDQVSNLVECL

SEQ ID NO: 16 - protein, 618 aa, synthetic construct
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIPTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLER

SEQ ID NO: 17 - protein, 649 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSATNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATFEEEYDLERAQKAVNALFTSTNQLGLKTDVTDYHIDQVSNLVECL

SEQ ID NO: 18 - protein, 611 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSANNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLER

SEQ ID NO: 19 - protein, 648 aa, synthetic construct
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIPTSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSARTNTIGPNRITQIPLVKALNLHSGVTGVRGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSD

SEQ ID NO: 20 - protein, 665 aa, synthetic construct
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIPTSAQLTREIYTDPIGRTNAPSGFASTNWFNNNAPSFSAIEAAIIRPPHLLDFPEQLTIYSASSRWSSTQHMIYWVGHRLNFRPIGGTLNTSTQGLTNNTSINPVTLQFTSRDVYRTESYAGTNILFTTPVNGVPWARFNFINPQNIYERGTTTYSQPYQGVGIQLFDSETELPPETTERPNYESYSHRLSHIGLIIGNTLRAPVYSWTHRSADTNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATFEEEYDLERAQKAVNALFTSTNQLGLKTDVTDYHIDQVSNLVECL

SEQ ID NO: 21 - protein, 665 aa, synthetic construct
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIPTSAQLTREIYTDPIGRTNAPSGFASTNWFNNNAPSFSAIEAAIIRPPHLLDFPEQLTIYSASSRWSSTQHMIYWVGHRLNFRPIGGTLNTSTQGLTNNTSINPVTLQFTSRDVYRTESYAGTNILFTTPVNGVPWARFNFINPQNIYERGTTTYSQPYQGVGIQLFDSETELPPETTERPNYESYSHRLSHIGLIIGNTLRAPVYSWTHRSADRTNTIGPNRITQIPLVKALNLHSGVTGVRGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSD

SEQ ID NO: 22 - protein, 673 aa, synthetic construct
MTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNQEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSATNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATFEEEYDLERAQKAVNALFTSTNQLGLKTDVTDYHIDQVSNLVECL

SEQ ID NO: 23 - protein, 673 aa, synthetic construct
MTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNQEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSARTNTIGPNRITQIPLVKALNLHSGVTGVRGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSD

SEQ ID NO: 24 - protein, 690 aa, synthetic construct
MTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNQEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTSAQLTREIYTDPIGRTNAPSGFASTNWFNNNAPSFSAIEAAIIRPPHLLDFPEQLTIYSASSRWSSTQHMIYWVGHRLNFRPIGGTLNTSTQGLTNNTSINPVTLQFTSRDVYRTESYAGTNILFTTPVNGVPWARFNFINPQNIYERGTTTYSQPYQGVGIQLFDSETELPPETTERPNYESYSHRLSHIGLIIGNTLRAPVYSWTHRSADTNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATFEEEYDLERAQKAVNALFTSTNQLGLKTDVTDYHIDQVSNLVECL

SEQ ID NO: 25 - protein, 613 aa, synthetic construct
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSANNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLER

SEQ ID NO: 26 - protein, 672 aa, synthetic construct
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFTLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 27 - protein, 621 aa, synthetic construct
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLER

SEQ ID NO: 28 - protein, 662 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 29 - protein, 611 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSANNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLER

SEQ ID NO: 30 - protein, 670 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFTLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 31 - protein, 639 aa, synthetic construct
MKNNIQNQCVPYNCLSNPEVEILSEERSTGRLPLDISLSLTRFLLSEFVPGVGVAFGLFDLIWGFITPSEWSLFLLQIEQLIEQRIETLERNRAITTLRGLADSYEVYLEALREWEENPNNAQLREDVRIRFANTDDALITAINNFTLTSFEIPLLSVYVQAANLHLSLLRDAVSFGQGWGLDIATVNNHYNRLINLIHRYTEHCLDTYNQGLENLRGTNTRQWSRFNQFRRELTLTVLDIVALFPNYDARAYPIQTATQLSREVYLDLPFINENLSPAASYPTFSAAESAIIRSPHLVDFLNNFTIYTDSLASYAYWGGHRVRSFRTGTTTNLIQSPLYGREGNTERPVTIWASSSVPIFRTLSYVTGLNNRNPVAGIEGVEFQDTTGRSIYRKSGPIDSFSELPPQDANISPTIGYSHRLCHATFLERISGPRIAGVVFSWTHRSASPTNEVSPSRITQIPWVKANTLASGASVIKGPGFTGGDILTRNSMGDLGSLRVTFTGRLPQSYYIRFRYASVANRSGSFRYSQPPSYGISFPKTMDAGEPLTSRSFALTTLFTPITFSRAQEEFDLYIQTGVYIDRIEFIPVTATLEAEYDLERAQEAVNALFTSSNQLGLKTDVTDYHIDQVSNLVDCLS

SEQ ID NO: 32 - protein, 644 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYARAFSDWEEDPTNPALREEMRIQFNDMNSALITAIPLFRVQNYEVALLSVYVQAANLHLSILRDISVFGERWGYDTATINNRYSDLTSLIHVYTNHCVDTYNQGLKLLEGRFLSDWIVYNRFRRQLTISVLDIVAFFPNYDIRAYPIQTATQLSREVYLDLPFINENLSPAASYPTFSAAESAIIRSPHLVDFLNNFTIYTDSLASYAYWGGHRVRSFRTGTTTNLIQSPLYGREGNTERPVTIWASSSVPIFRTLSYVTGLNNRNPVAGIEGVEFQDTTGRSIYRKSGPIDSFSELPPQDANISPTIGYSHRLCHATFLERISGPRIAGVVFSWTHRSASPTNEVSPSRITQIPAVKAHNLHSGSTVVRGPGFTGGDLLRRTNTGTFADIRVNITGPLSQRYRVRIRYASTTDLQFFTRINGTSVNQGNFQRTMNRGGNLESGNFRTAGFSTPFSFSNAQSTFTLGTQAFSNQEVYIDRIEFVPAEVTFEAESDLERAQKAVNALFTSTSQLGLKTNVTGYHIDQVSNLVACLS

SEQ ID NO: 33 - protein, 645 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYARAFSDWEEDPTNPALREEMRIQFNDMNSALITAIPLFRVQNYEVALLSVYVQAANLHLSILRDISVFGERWGYDTATINNRYSDLTSLIHVYTNHCVDTYNQGLKLLEGRFLSDWIVYNRFRRQLTISVLDIVAFFPNYDIRAYPIQTSSQLTREIYTSSVIEDSPVSANIPNGFNRAEFGVRPPHLMDFMNSLFVTAETVRSQTVWGGHLVSSRNTAGNPINFPIYGIFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEIAGSDSWRAPMFSWTHRSADRTNIINPNIITQIPWVKANTLASGASVIKGPGFTGGDILTRNSMGDLGSLRVTFTGRLPQSYYIRFRYASVANRSGSFRYSQPPSYGISFPKTMDAGEPLTSRSFALTTLFTPITFSRAQEEFDLYIQTGVYIDRIEFIPVTATLEAEYDLERAQEAVNALFTSSNQLGLKTDVTDYHIDQVSNLVDCLS

SEQ ID NO: 34 - protein, 649 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYARAFSDWEEDPTNPALREEMRIQFNDMNSALITAIPLFRVQNYEVALLSVYVQAANLHLSILRDISVFGERWGYDTATINNRYSDLTSLIHVYTNHCVDTYNQGLKLLEGRFLSDWIVYNRFRRQLTISVLDIVAFFPNYDIRAYPIQTSSQLTREIYTSSVIEDSPVSANIPNGFNRAEFGVRPPHLMDFMNSLFVTAETVRSQTVWGGHLVSSRNTAGNPINFPIYGIFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEIAGSDSWRAPMFSWTHRSADRTNIINPNIITQIPAVKAHNLHSGSTVVRGPGFTGGDLLRRTNTGTFADIRVNITGPLSQRYRVRIRYASTTDLQFFTRINGTSVNQGNFQRTMNRGGNLESGNFRTAGFSTPFSFSNAQSTFTLGTQAFSNQEVYIDRIEFVPAEVTFEAESDLERAQKAVNALFTSTSQLGLKTNVTGYHIDQVSNLVACLS

SEQ ID NO: 35 - protein, 643 aa, synthetic construct
MKNNIQNQCVPYNCLSNPEVEILSEERSTGRLPLDISLSLTRFLLSEFVPGVGVAFGLFDLIWGFITPSEWSLFLLQIEQLIEQRIETLERNRAITTLRGLADSYEVYLEALREWEENPNNAQLREDVRIRFANTDDALITAINNFTLTSFEIPLLSVYVQAANLHLSLLRDAVSFGQGWGLDIATVNNHYNRLINLIHRYTEHCLDTYNQGLENLRGTNTRQWSRFNQFRRELTLTVLDIVALFPNYDARAYPIQTATQLSREVYLDLPFINENLSPAASYPTFSAAESAIIRSPHLVDFLNNFTIYTDSLASYAYWGGHRVRSFRTGTTTNLIQSPLYGREGNTERPVTIWASSSVPIFRTLSYVTGLNNRNPVAGIEGVEFQDTTGRSIYRKSGPIDSFSELPPQDANISPTIGYSHRLCHATFLERISGPRIAGVVFSWTHRSASPTNEVSPSRITQIPAVKAHNLHSGSTVVRGPGFTGGDLLRRTNTGTFADIRVNITGPLSQRYRVRIRYASTTDLQFFTRINGTSVNQGNFQRTMNRGGNLESGNFRTAGFSTPFSFSNAQSTFTLGTQAFSNQEVYIDRIEFVPAEVTFEAESDLERAQKAVNALFTSTSQLGLKTNVTGYHIDQVSNLVACLS

SEQ ID NO: 36 - protein, 644 aa, synthetic construct
MKNNIQNQCVPYNCLSNPEVEILSEERSTGRLPLDISLSLTRFLLSEFVPGVGVAFGLFDLIWGFITPSEWSLFLLQIEQLIEQRIETLERNRAITTLRGLADSYEVYLEALREWEENPNNAQLREDVRIRFANTDDALITAINNFTLTSFEIPLLSVYVQAANLHLSLLRDAVSFGQGWGLDIATVNNHYNRLINLIHRYTEHCLDTYNQGLENLRGTNTRQWSRFNQFRRELTLTVLDIVALFPNYDARAYPIQTSSQLTREIYTSSVIEDSPVSANIPNGFNRAEFGVRPPHLMDFMNSLFVTAETVRSQTVWGGHLVSSRNTAGNPINFPIYGIFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEIAGSDSWRAPMFSWTHRSADRTNIINPNIITQIPWVKANTLASGASVIKGPGFTGGDILTRNSMGDLGSLRVTFTGRLPQSYYIRFRYASVANRSGSFRYSQPPSYGISFPKTMDAGEPLTSRSFALTTLFTPITFSRAQEEFDLYIQTGVYIDRIEFIPVTATLEAEYDLERAQEAVNALFTSSNQLGLKTDVTDYHIDQVSNLVDCLS

SEQ ID NO: 37 - protein, 661 aa, synthetic construct
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIPTSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 38 - protein, 648 aa, synthetic construct
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIPQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATFEEEYDLERAQKAVNALFTSTNQLGLKTDVTDYHIDQVSNLVECL

SEQ ID NO: 39 - protein, 661 aa, synthetic construct
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIPQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 40 - protein, 648 aa, synthetic construct
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIPTSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSATNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATFEEEYDLERAQKAVNALFTSTNQLGLKTDVTDYHIDQVSNLVECL

SEQ ID NO: 41 - protein, 664 aa, synthetic construct
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGTSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 42 - protein, 651 aa, synthetic construct
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATFEEEYDLERAQKAVNALFTSTNQLGLKTDVTDYHIDQVSNLVECL

SEQ ID NO: 43 - protein, 701 aa, synthetic construct
MNSVLNSGRTTICDAYNVVAHDPFSFEHKSLDTIQKEWMEWKRTDHSLYVAPVVGTVSSFLLKKVGSLIGKRILSELWGIIFPSGSTNLMQDILRETEQFLNQRLNTDTLARVNAELIGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSLMVSSGANLYASGSGPQQTQSFTAQNWPFLYSLFQVNSNYILSGISGTRLSITFPNIGGLPGSTTTHSLNSARVNYSGGVSSGLIGATNLNHNFNCSTVLPPLSTPFVRSWLDSGTDREGVATSTNWQTESFQTTLSLRCGAFSARGNSNYFPDYFIRNISGVPLVIRNEDLTRPLHYNQIRNIESPSGTPGGARAYLVSVHNRKNNIYAANENGTMIHLAPEDYTGTLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 44 - protein, 623 aa, synthetic construct
MNSVLNSGRTTICDAYNVVAHDPFSFEHKSLDTIQKEWMEWKRTDHSLYVAPVVGTVSSFLLKKVGSLIGKRILSELWGIIFPSGSTNLMQDILRETEQFLNQRLNTDTLARVNAELIGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSANIYAANENGTMIHLAPEDYTGFTISPIHATQVNNQTRTFISEKFGNQGDSLRFEQSNTTARYTLRGNGNSYNLYLRVSSIGNSTIRVTINGRVYTVSNVNTTTNNDGVNDNGARFSDINIGNIVASDNTNVTLDINVTLNSGTPFDLMNIMFVPTNLPPLY

SEQ ID NO: 45 - protein, 670 aa, synthetic construct
MNSVLNSGRTTICDAYNVVAHDPFSFEHKSLDTIQKEWMEWKRTDHSLYVAPVVGTVSSFLLKKVGSLIGKRILSELWGIIFPSGSTNLMQDILRETEQFLNQRLNTDTLARVNAELIGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 46 - protein, 627 aa, synthetic construct
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGLMVSSGANLYASGSGPQQTQSFTAQNWPFLYSLFQVNSNYILSGISGTRLSITFPNIGGLPGSTTTHSLNSARVNYSGGVSSGLIGATNLNHNFNCSTVLPPLSTPFVRSWLDSGTDREGVATSTNWQTESFQTTLSLRCGAFSARGNSNYFPDYFIRNISGVPLVIRNEDLTRPLHYNQIRNIESPSGTPGGARAYLVSVHNRKNNIYAANENGTMIHLAPEDYTGFTISPIHATQVNNQTRTFISEKFGNQGDSLRFEQSNTTARYTLRGNGNSYNLYLRVSSIGNSTIRVTINGRVYTVSNVNTTTNNDGVNDNGARFSDINIGNIVASDNTNVTLDINVTLNSGTPFDLMNIMFVPTNLPPLY

SEQ ID NO: 47 - protein, 695 aa, synthetic construct
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGLMVSSGANLYASGSGPQQTQSFTAQNWPFLYSLFQVNSNYILSGISGTRLSITFPNIGGLPGSTTTHSLNSARVNYSGGVSSGLIGATNLNHNFNCSTVLPPLSTPFVRSWLDSGTDREGVATSTNWQTESFQTTLSLRCGAFSARGNSNYFPDYFIRNISGVPLVIRNEDLTRPLHYNQIRNIESPSGTPGGARAYLVSVHNRKNNIYAANENGTMIHLAPEDYTGTLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 48 - protein, 596 aa, synthetic construct
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSAFTISPIHATQVNNQTRTFISEKFGNQGDSLRFEQSNTTARYTLRGNGNSYNLYLRVSSIGNSTIRVTINGRVYTVSNVNTTTNNDGVNDNGARFSDINIGNIVASDNTNVTLDINVTLNSGTPFDLMNIMFVPTNLPPLY

SEQ ID NO: 49 - DNA, 1953 bp, synthetic construct
  source 1..1953: mol_type = other DNA
atggaggagaacaaccagaaccagtgcatcccgtacaactgcctgtccaacccggaggaggtgctgctggacggcgagaggatctccaccggcaactcctccatcgacatctccctgtccctggtgcagttcctggtgtccaacttcgtgccgggcggcggcttcctggtgggcctgatcgacttcgtgtggggcatcgtgggcccgtcccagtgggacgccttcctggtgcagatcgagcagctgatcaacgagaggatcgccgagttcgccaggaacgccgccatcgccaacctggagggcctgggcaacaacttcaacatctacgtggaggccttcaaggagtgggaggaggacccgaacaacccggccaccaggaccagggtgatcgacaggttcaggatcctggacggcctgctggagagggacatcccgtccttcaggatctccggcttcgaggtgccgctgctgtccgtgtacgcccaggccgccaacctgcacctggccatcctgagggactccgtgatcttcggcgagaggtggggcctgaccaccatcaacgtgaacgagaactacaacaggctgatcaggcacatcgacgagtacgccgaccactgcgccaacacctacaacaggggcctgaacaacctgccgaagtccacctaccaggactggatcacctacaacaggctgaggagggacctgaccctgaccgtgctggacatcgccgccttcttcccgaactacgacaacaggaggtacccgatccagccggtgggccagctgaccagggaggtgtacaccgacccgctgatcaacttcaacccgcagctgcagtccgtggcccagctgccgaccttcaacgtgatggagtcctccgccatcaggaacccgcacctgttcgacatcctgaacaacctgaccatcttcaccgactggttctccgtgggcaggaacttctactggggcggccacagggtgatctcctccctgatcggcggcggcaacatcacctccccgatctacggcagggaggccaaccaggagccgccgaggtccttcaccttcaacggcccggtgttcaggaccctgtccaacccgaccctgaggctgctgcagcagccgtggccggccccgccgttcaacctgaggggcgtggagggcgtggagttctccaccccgaccaactccttcacctacaggggcaggggcaccgtggactccctgaccgagctgccgccggaggacaactccgtgccgccgagggagggctactcccacaggctgtgccacgccaccttcgtgcagaggtccggcaccccgttcctgaccaccggcgtggtgttctcctggacccacaggtccgccaggaccaacaccatcggcccgaacaggatcacccagatcccgctggtgaaggccctgaacctgcactccggcgtgaccggcgtgaggggcccgggcttcaccggcggcgacatcctgaggaggaccaacaccggcaccttcggcgacatcaggctgaacatcaacgtgccgctgtcccagaggtacagggtgaggatcaggtacgcctccaccaccgacctgcagttcttcaccaggatcaacggcaccaccgtgaacatcggcaacttctccaggaccatgaacaggggcgacaacctggagtacaggtccttcaggaccgccggcttctccaccccgttcaacttcctgaacgcccagtccaccttcaccctgggcgcccagtccttctccaaccaggaggtgtacatcgacagggtggagttcgtgccggccgaggtgaccttcgaggccgagtacgacctggagagggcccagaaggccgtgaacgccctgttcacctccaccaacccgaggaggctgaagaccgacgtgaccgactaccacatcgaccaggtgtccaacatggtggcctgcctgtccgac

SEQ ID NO: 50 - DNA, 2043 bp, synthetic construct
  source 1..2043: mol_type = other DNA
atggaggagaacaaccagaaccagtgcatcccgtacaactgcctgtccaacccggaggaggtgctgctggacggcgagaggatctccaccggcaactcctccatcgacatctccctgtccctggtgcagttcctggtgtccaacttcgtgccgggcggcggcttcctggtgggcctgatcgacttcgtgtggggcatcgtgggcccgtcccagtgggacgccttcctggtgcagatcgagcagctgatcaacgagaggatcgccgagttcgccaggaacgccgccatcgccaacctggagggcctgggcaacaacttcaacatctacgtggaggccttcaaggagtgggaggaggacccgaacaacccggccaccaggaccagggtgatcgacaggttcaggatcctggacggcctgctggagagggacatcccgtccttcaggatctccggcttcgaggtgccgctgctgtccgtgtacgcccaggccgccaacctgcacctggccatcctgagggactccgtgatcttcggcgagaggtggggcctgaccaccatcaacgtgaacgagaactacaacaggctgatcaggcacatcgacgagtacgccgaccactgcgccaacacctacaacaggggcctgaacaacctgccgaagtccacctaccaggactggatcacctacaacaggctgaggagggacctgaccctgaccgtgctggacatcgccgccttcttcccgaactacgacaacaggaggtacccgatccagccggtgggcacctccgcccagctgaccagggagatctacaccgacccgatcggcaggaccaacgccccgtccggcttcgcctccaccaactggttcaacaacaacgccccgtccttctccgccatcgaggccgccatcatcaggccgccgcacctgctggacttcccggagcagctgaccatctactccgcctcctccaggtggtcctccacccagcacatgatctactgggtgggccacaggctgaacttcaggccgatcggcggcaccctgaacacctccacccagggcctgaccaacaacacctccatcaacccggtgaccctgcagttcacctccagggacgtgtacaggaccgagtcctacgccggcaccaacatcctgttcaccaccccggtgaacggcgtgccgtgggccaggttcaacttcatcaacccgcagaacatctacgagaggggcaccaccacctactcccagccgtaccagggcgtgggcatccagctgttcgactccgagaccgagctgccgccggagaccaccgagaggccgaactacgagtcctactcccacaggctgtcccacatcggcctgatcatcggcaacaccctgagggccccggtgtactcctggacccacaggtccgccgacaccctgaccaacaccatcgacccggagaggatcaaccagatcccgctggtgaagggcttcagggtgtggggcggcacctccgtgatcaccggcccgggcttcaccggcggcgacatcctgaggaggaacaccttcggcgacttcgtgtccctgcaggtgaacatcaactccccgatcacccagaggtacaggctgaggttcaggtacgcctcctccagggacgccagggtgatcgtgctgaccggcgccgcctccaccggcgtgggcggccaggtgtccgtgaacatgccgctgcagaagaccatggagatcggcgagaacctgacctccaggaccttcaggtacaccgacttctccaacccgttctccttcagggccaacccggacatcatcggcatctccgagcagccgctgttcggcgccggctccatctcctccggcgagctgtacatcgacaagatcgagatcatcctggccgacgccaccttcgaggccgagtccgacctggagagggcccagaaggccgtgaacgccctgttcacctcctccaaccagatcggcctgaagaccgacgtgaccgactaccacatcgaccaggtgtccaacctggtggactgcctgtcc

SEQ ID NO: 51 - DNA, 2004 bp, synthetic construct
  source 1..2004: mol_type = other DNA
atggaggagaacaaccagaaccagtgcatcccgtacaactgcctgtccaacccggaggaggtgctgctggacggcgagaggatctccaccggcaactcctccatcgacatctccctgtccctggtgcagttcctggtgtccaacttcgtgccgggcggcggcttcctggtgggcctgatcgacttcgtgtggggcatcgtgggcccgtcccagtgggacgccttcctggtgcagatcgagcagctgatcaacgagaggatcgccgagttcgccaggaacgccgccatcgccaacctggagggcctgggcaacaacttcaacatctacgtggaggccttcaaggagtgggaggaggacccgaacaacccggccaccaggaccagggtgatcgacaggttcaggatcctggacggcctgctggagagggacatcccgtccttcaggatctccggcttcgaggtgccgctgctgtccgtgtacgcccaggccgccaacctgcacctggccatcctgagggactccgtgatcttcggcgagaggtggggcctgaccaccatcaacgtgaacgagaactacaacaggctgatcaggcacatcgacgagtacgccgaccactgcgccaacacctacaacaggggcctgaacaacctgccgaagtccacctaccaggactggatcacctacaacaggctgaggagggacctgaccctgaccgtgctggacatcgccgccttcttcccgaactacgacaacaggaggtacccgatccagccggtgggcacctccgcccagctgaccagggagatctacaccgacccgatcggcaggaccaacgccccgtccggcttcgcctccaccaactggttcaacaacaacgccccgtccttctccgccatcgaggccgccatcatcaggccgccgcacctgctggacttcccggagcagctgaccatctactccgcctcctccaggtggtcctccacccagcacatgatctactgggtgggccacaggctgaacttcaggccgatcggcggcaccctgaacacctccacccagggcctgaccaacaacacctccatcaacccggtgaccctgcagttcacctccagggacgtgtacaggaccgagtcctacgccggcaccaacatcctgttcaccaccccggtgaacggcgtgccgtgggccaggttcaacttcatcaacccgcagaacatctacgagaggggcaccaccacctactcccagccgtaccagggcgtgggcatccagctgttcgactccgagaccgagctgccgccggagaccaccgagaggccgaactacgagtcctactcccacaggctgtcccacatcggcctgatcatcggcaacaccctgagggccccggtgtactcctggacccacaggtccgccgacaggaccaacaccatcggcccgaacaggatcacccagatcccgctggtgaaggccctgaacctgcactccggcgtgaccggcgtgaggggcccgggcttcaccggcggcgacatcctgaggaggaccaacaccggcaccttcggcgacatcaggctgaacatcaacgtgccgctgtcccagaggtacagggtgaggatcaggtacgcctccaccaccgacctgcagttcttcaccaggatcaacggcaccaccgtgaacatcggcaacttctccaggaccatgaacaggggcgacaacctggagtacaggtccttcaggaccgccggcttctccaccccgttcaacttcctgaacgcccagtccaccttcaccctgggcgcccagtccttctccaaccaggaggtgtacatcgacagggtggagttcgtgccggccgaggtgaccttcgaggccgagtacgacctggagagggcccagaaggccgtgaacgccctgttcacctccaccaacccgaggaggctgaagaccgacgtgaccgactaccacatcgaccaggtgtccaacatggtggcctgcctgtccgac

SEQ ID NO: 52 - DNA, 2058 bp, synthetic construct
  source 1..2058: mol_type = other DNA
atgacctccaacaggaagaacgagaacgagatcatcaacgccctgtccatcccgaccgtgtccaacccgtccacccagatgaacctgtccccggacgccaggatcgaggactccctgtgcgtggccgaggtgaacaacatcgacccgttcgtgtccgcctccaccgtgcagaccggcatcaacatcgccggcaggatcctgggcgtgctgggcgtgccgttcgccggccagctggcctccttctactccttcctggtgggcgagctgtggccgtccggcagggacccgtgggagatcttcctggagcacgtggagcagctgatcaggcagcaggtgaccgagaacaccaggaacaccgccatcgccaggctggagggcctgggcaggggctacaggtcctaccagcaggccctggagacctggctggacaacaggaacgacgccaggtccaggtccatcatcctggagaggtacgtggccctggagctggacatcaccaccgccatcccgctgttcaggatcaggaaccaggaggtgccgctgctgatggtgtacgcccaggccgccaacctgcacctgctgctgctgagggacgcctccctgttcggctccgagtggggcatggcctcctccgacgtgaaccagtactaccaggagcagatcaggtacaccgaggagtactccaaccactgcgtgcagtggtacaacaccggcctgaacaacctgaggggcaccaacgccgagtcctggctgaggtacaaccagttcaggagggacctgaccctgggcgtgctggacctggtggccctgttcccgtcctacgacaccaggacctacccgatcaaccagctgaccagggaggtgtacaccgacccgctgatcaacttcaacccgcagctgcagtccgtggcccagctgccgaccttcaacgtgatggagtcctccgccatcaggaacccgcacctgttcgacatcctgaacaacctgaccatcttcaccgactggttctccgtgggcaggaacttctactggggcggccacagggtgatctcctccctgatcggcggcggcaacatcacctccccgatctacggcagggaggccaaccaggagccgccgaggtccttcaccttcaacggcccggtgttcaggaccctgtccaacccgaccctgaggctgctgcagcagccgtggccggccccgccgttcaacctgaggggcgtggagggcgtggagttctccaccccgaccaactccttcacctacaggggcaggggcaccgtggactccctgaccgagctgccgccggaggacaactccgtgccgccgagggagggctactcccacaggctgtgccacgccaccttcgtgcagaggtccggcaccccgttcctgaccaccggcgtggtgttctcctggacccacaggtccgccaccctgaccaacaccatcgacccggagaggatcaaccagatcccgctggtgaagggcttcagggtgtggggcggcacctccgtgatcaccggcccgggcttcaccggcggcgacatcctgaggaggaacaccttcggcgacttcgtgtccctgcaggtgaacatcaactccccgatcacccagaggtacaggctgaggttcaggtacgcctcctccagggacgccagggtgatcgtgctgaccggcgccgcctccaccggcgtgggcggccaggtgtccgtgaacatgccgctgcagaagaccatggagatcggcgagaacctgacctccaggaccttcaggtacaccgacttctccaacccgttctccttcagggccaacccggacatcatcggcatctccgagcagccgctgttcggcgccggctccatctcctccggcgagctgtacatcgacaagatcgagatcatcctggccgacgccaccttcgaggccgagtccgacctggagagggcccagaaggccgtgaacgccctgttcacctcctccaaccagatcggcctgaagaccgacgtgaccgactaccacatcgaccaggtgtccaacctggtggactgcctgtcc

SEQ ID NO: 53 - DNA, 2019 bp, synthetic construct
  source 1..2019: mol_type = other DNA
atgacctccaacaggaagaacgagaacgagatcatcaacgccctgtccatcccgaccgtgtccaacccgtccacccagatgaacctgtccccggacgccaggatcgaggactccctgtgcgtggccgaggtgaacaacatcgacccgttcgtgtccgcctccaccgtgcagaccggcatcaacatcgccggcaggatcctgggcgtgctgggcgtgccgttcgccggccagctggcctccttctactccttcctggtgggcgagctgtggccgtccggcagggacccgtgggagatcttcctggagcacgtggagcagctgatcaggcagcaggtgaccgagaacaccaggaacaccgccatcgccaggctggagggcctgggcaggggctacaggtcctaccagcaggccctggagacctggctggacaacaggaacgacgccaggtccaggtccatcatcctggagaggtacgtggccctggagctggacatcaccaccgccatcccgctgttcaggatcaggaaccaggaggtgccgctgctgatggtgtacgcccaggccgccaacctgcacctgctgctgctgagggacgcctccctgttcggctccgagtggggcatggcctcctccgacgtgaaccagtactaccaggagcagatcaggtacaccgaggagtactccaaccactgcgtgcagtggtacaacaccggcctgaacaacctgaggggcaccaacgccgagtcctggctgaggtacaaccagttcaggagggacctgaccctgggcgtgctggacctggtggccctgttcccgtcctacgacaccaggacctacccgatcaaccagctgaccagggaggtgtacaccgacccgctgatcaacttcaacccgcagctgcagtccgtggcccagctgccgaccttcaacgtgatggagtcctccgccatcaggaacccgcacctgttcgacatcctgaacaacctgaccatcttcaccgactggttctccgtgggcaggaacttctactggggcggccacagggtgatctcctccctgatcggcggcggcaacatcacctccccgatctacggcagggaggccaaccaggagccgccgaggtccttcaccttcaacggcccggtgttcaggaccctgtccaacccgaccctgaggctgctgcagcagccgtggccggccccgccgttcaacctgaggggcgtggagggcgtggagttctccaccccgaccaactccttcacctacaggggcaggggcaccgtggactccctgaccgagctgccgccggaggacaactccgtgccgccgagggagggctactcccacaggctgtgccacgccaccttcgtgcagaggtccggcaccccgttcctgaccaccggcgtggtgttctcctggacccacaggtccgccaggaccaacaccatcggcccgaacaggatcacccagatcccgctggtgaaggccctgaacctgcactccggcgtgaccggcgtgaggggcccgggcttcaccggcggcgacatcctgaggaggaccaacaccggcaccttcggcgacatcaggctgaacatcaacgtgccgctgtcccagaggtacagggtgaggatcaggtacgcctccaccaccgacctgcagttcttcaccaggatcaacggcaccaccgtgaacatcggcaacttctccaggaccatgaacaggggcgacaacctggagtacaggtccttcaggaccgccggcttctccaccccgttcaacttcctgaacgcccagtccaccttcaccctgggcgcccagtccttctccaaccaggaggtgtacatcgacagggtggagttcgtgccggccgaggtgaccttcgaggccgagtacgacctggagagggcccagaaggccgtgaacgccctgttcacctccaccaacccgaggaggctgaagaccgacgtgaccgactaccacatcgaccaggtgtccaacatggtggcctgcctgtccgac

SEQ ID NO: 54 - DNA, 2109 bp, synthetic construct
  source 1..2109: mol_type = other DNA
atgacctccaacaggaagaacgagaacgagatcatcaacgccctgtccatcccgaccgtgtccaacccgtccacccagatgaacctgtccccggacgccaggatcgaggactccctgtgcgtggccgaggtgaacaacatcgacccgttcgtgtccgcctccaccgtgcagaccggcatcaacatcgccggcaggatcctgggcgtgctgggcgtgccgttcgccggccagctggcctccttctactccttcctggtgggcgagctgtggccgtccggcagggacccgtgggagatcttcctggagcacgtggagcagctgatcaggcagcaggtgaccgagaacaccaggaacaccgccatcgccaggctggagggcctgggcaggggctacaggtcctaccagcaggccctggagacctggctggacaacaggaacgacgccaggtccaggtccatcatcctggagaggtacgtggccctggagctggacatcaccaccgccatcccgctgttcaggatcaggaaccaggaggtgccgctgctgatggtgtacgcccaggccgccaacctgcacctgctgctgctgagggacgcctccctgttcggctccgagtggggcatggcctcctccgacgtgaaccagtactaccaggagcagatcaggtacaccgaggagtactccaaccactgcgtgcagtggtacaacaccggcctgaacaacctgaggggcaccaacgccgagtcctggctgaggtacaaccagttcaggagggacctgaccctgggcgtgctggacctggtggccctgttcccgtcctacgacaccaggacctacccgatcaacacctccgcccagctgaccagggagatctacaccgacccgatcggcaggaccaacgccccgtccggcttcgcctccaccaactggttcaacaacaacgccccgtccttctccgccatcgaggccgccatcatcaggccgccgcacctgctggacttcccggagcagctgaccatctactccgcctcctccaggtggtcctccacccagcacatgatctactgggtgggccacaggctgaacttcaggccgatcggcggcaccctgaacacctccacccagggcctgaccaacaacacctccatcaacccggtgaccctgcagttcacctccagggacgtgtacaggaccgagtcctacgccggcaccaacatcctgttcaccaccccggtgaacggcgtgccgtgggccaggttcaacttcatcaacccgcagaacatctacgagaggggcaccaccacctactcccagccgtaccagggcgtgggcatccagctgttcgactccgagaccgagctgccgccggagaccaccgagaggccgaactacgagtcctactcccacaggctgtcccacatcggcctgatcatcggcaacaccctgagggccccggtgtactcctggacccacaggtccgccgacaccctgaccaacaccatcgacccggagaggatcaaccagatcccgctggtgaagggcttcagggtgtggggcggcacctccgtgatcaccggcccgggcttcaccggcggcgacatcctgaggaggaacaccttcggcgacttcgtgtccctgcaggtgaacatcaactccccgatcacccagaggtacaggctgaggttcaggtacgcctcctccagggacgccagggtgatcgtgctgaccggcgccgcctccaccggcgtgggcggccaggtgtccgtgaacatgccgctgcagaagaccatggagatcggcgagaacctgacctccaggaccttcaggtacaccgacttctccaacccgttctccttcagggccaacccggacatcatcggcatctccgagcagccgctgttcggcgccggctccatctcctccggcgagctgtacatcgacaagatcgagatcatcctggccgacgccaccttcgaggccgagtccgacctggagagggcccagaaggccgtgaacgccctgttcacctcctccaaccagatcggcctgaagaccgacgtgaccgactaccacatcgaccaggtgtccaacctggtggactgcctgtcc

SEQ ID NO: 55 - DNA, 1971 bp, synthetic construct
  source 1..1971: mol_type = other DNA
atggacaacaacccgaacatcaacgagtgcatcccgtacaactgcctgtccaacccggaggtggaggtgctgggcggcgagaggatcgagaccggctacaccccgatcgacatctccctgtccctgacccagttcctgctgtccgagttcgtgccgggcgccggcttcgtgctgggcctggtggacatcatctggggcatcttcggcccgtcccagtgggacgccttcctggtgcagatcgagcagctgatcaaccagaggatcgaggagttcgccaggaaccaggccatctccaggctggagggcctgtccaacctgtaccagatctacgccgagtccttcagggagtgggaggccgacccgaccaacccggccctgagggaggagatgaggatccagttcaacgacatgaactccgccctgaccaccgccatcccgctgttcgccgtgcagaactaccaggtgccgctgctgtccgtgtacgtgcaggccgccaacctgcacctgtccgtgctgagggacgtgtccgtgttcggccagaggtggggcttcgacgccgccaccatcaactccaggtacaacgacctgaccaggctgatcggcaactacaccgaccacgccgtgaggtggtacaacaccggcctggagagggtgtggggcccggactccagggactggatcaggtacaaccagttcaggagggagctgaccctgaccgtgctggacatcgtgtccctgttcccgaactacgactccaggacctacccgatcaggaccgtgtcccagctgaccagggagatctacaccaacccggtgctggagaacttcgacggctccttcaggggctccgcccagggcatcgagggctccatcaggtccccgcacctgatggacatcctgaactccatcaccatctacaccgacgcccacaggggcgagtactactggtccggccaccagatcatggcctccccggtgggcttctccggcccggagttcaccttcccgctgtacggcaccatgggcaacgccgccccgcagcagaggatcgtggcccagctgggccagggcgtgtacaggaccctgtcctccaccctgtacaggaggccgttcaacatcggcatcaacaaccagcagctgtccgtgctggacggcaccgagttcgcctacggcacctcctccaacctgccgtccgccgtgtacaggaagtccggcaccgtggactccctggacgagatcccgccgcagaacaacaacgtgccgccgaggcagggcttctcccacaggctgtcccacgtgtccatgttcaggtccggcttctccaactcctccgtgtccatcatcagggccccgatgttctcctggatccacaggtccgccgagttcaggaccaacaccatcggcccgaacaggatcacccagatcccgctggtgaaggccctgaacctgcactccggcgtgaccggcgtgaggggcccgggcttcaccggcggcgacatcctgaggaggaccaacaccggcaccttcggcgacatcaggctgaacatcaacgtgccgctgtcccagaggtacagggtgaggatcaggtacgcctccaccaccgacctgcagttcttcaccaggatcaacggcaccaccgtgaacatcggcaacttctccaggaccatgaacaggggcgacaacctggagtacaggtccttcaggaccgccggcttctccaccccgttcaacttcctgaacgcccagtccaccttcaccctgggcgcccagtccttctccaaccaggaggtgtacatcgacagggtggagttcgtgccggccgaggtgaccttcgaggccgagtacgacctggagagggcccagaaggccgtgaacgccctgttcacctccaccaacccgaggaggctgaagaccgacgtgaccgactaccacatcgaccaggtgtccaacatggtggcctgcctgtccgac

SEQ ID NO: 56 - DNA, 1998 bp, synthetic construct
  source 1..1998: mol_type = other DNA
atggacaacaacccgaacatcaacgagtgcatcccgtacaactgcctgtccaacccggaggtggaggtgctgggcggcgagaggatcgagaccggctacaccccgatcgacatctccctgtccctgacccagttcctgctgtccgagttcgtgccgggcgccggcttcgtgctgggcctggtggacatcatctggggcatcttcggcccgtcccagtgggacgccttcctggtgcagatcgagcagctgatcaaccagaggatcgaggagttcgccaggaaccaggccatctccaggctggagggcctgtccaacctgtaccagatctacgccgagtccttcagggagtgggaggccgacccgaccaacccggccctgagggaggagatgaggatccagttcaacgacatgaactccgccctgaccaccgccatcccgctgttcgccgtgcagaactaccaggtgccgctgctgtccgtgtacgtgcaggccgccaacctgcacctgtccgtgctgagggacgtgtccgtgttcggccagaggtggggcttcgacgccgccaccatcaactccaggtacaacgacctgaccaggctgatcggcaactacaccgaccacgccgtgaggtggtacaacaccggcctggagagggtgtggggcccggactccagggactggatcaggtacaaccagttcaggagggagctgaccctgaccgtgctggacatcgtgtccctgttcccgaactacgactccaggacctacccgatcaggacctccgcccagctgaccagggagatctacaccgacccgatcggcaggaccaacgccccgtccggcttcgcctccaccaactggttcaacaacaacgccccgtccttctccgccatcgaggccgccatcatcaggccgccgcacctgctggacttcccggagcagctgaccatctactccgcctcctccaggtggtcctccacccagcacatgatctactgggtgggccacaggctgaacttcaggccgatcggcggcaccctgaacacctccacccagggcctgaccaacaacacctccatcaacccggtgaccctgcagttcacctccagggacgtgtacaggaccgagtcctacgccggcaccaacatcctgttcaccaccccggtgaacggcgtgccgtgggccaggttcaacttcatcaacccgcagaacatctacgagaggggcaccaccacctactcccagccgtaccagggcgtgggcatccagctgttcgactccgagaccgagctgccgccggagaccaccgagaggccgaactacgagtcctactcccacaggctgtcccacatcggcctgatcatcggcaacaccctgagggccccggtgtactcctggacccacaggtccgccgacaacaacatcatcccgtcctcccagatcacccagatcccgctgaccaagtccaccaacctgggctccggcacctccgtggtgaagggcccgggcttcaccggcggcgacatcctgaggaggacctccccgggccagatctccaccctgagggtgaacatcaccgccccgctgtcccagaggtacagggtgaggatcaggtacgcctccaccaccaacctgcagttccacacctccatcgacggcaggccgatcaaccagggcaacttctccgccaccatgtcctccggctccaacctgcagtccggctccttcaggaccgtgggcttcaccaccccgttcaacttctccaacggctcctccgtgttcaccctgtccgcccacgtgttcaactccggcaacgaggtgtacatcgacaggatcgagttcgtgccggccgaggtgaccttcgaggccgagtacgacctggagagggcccagaaggccgtgaacgagctgttcacctcctccaaccagatcggcctgaagaccgacgtgaccgactaccacatcgaccaggtgtccaacctggtggagtgcctgtccgac

SEQ ID NO: 57 - DNA, 1998 bp, synthetic construct
  source 1..1998: mol_type = other DNA
atggacaacaacccgaacatcaacgagtgcatcccgtacaactgcctgtccaacccggaggtggaggtgctgggcggcgagaggatcgagaccggctacaccccgatcgacatctccctgtccctgacccagttcctgctgtccgagttcgtgccgggcgccggcttcgtgctgggcctggtggacatcatctggggcatcttcggcccgtcccagtgggacgccttcctggtgcagatcgagcagctgatcaaccagaggatcgaggagttcgccaggaaccaggccatctccaggctggagggcctgtccaacctgtaccagatctacgccgagtccttcagggagtgggaggccgacccgaccaacccggccctgagggaggagatgaggatccagttcaacgacatgaactccgccctgaccaccgccatcccgctgttcgccgtgcagaactaccaggtgccgctgctgtccgtgtacgtgcaggccgccaacctgcacctgtccgtgctgagggacgtgtccgtgttcggccagaggtggggcttcgacgccgccaccatcaactccaggtacaacgacctgaccaggctgatcggcaactacaccgaccacgccgtgaggtggtacaacaccggcctggagagggtgtggggcccggactccagggactggatcaggtacaaccagttcaggagggagctgaccctgaccgtgctggacatcgtgtccctgttcccgaactacgactccaggacctacccgatcaggacctccgcccagctgaccagggagatctacaccgacccgatcggcaggaccaacgccccgtccggcttcgcctccaccaactggttcaacaacaacgccccgtccttctccgccatcgaggccgccatcatcaggccgccgcacctgctggacttcccggagcagctgaccatctactccgcctcctccaggtggtcctccacccagcacatgatctactgggtgggccacaggctgaacttcaggccgatcggcggcaccctgaacacctccacccagggcctgaccaacaacacctccatcaacccggtgaccctgcagttcacctccagggacgtgtacaggaccgagtcctacgccggcaccaacatcctgttcaccaccccggtgaacggcgtgccgtgggccaggttcaacttcatcaacccgcagaacatctacgagaggggcaccaccacctactcccagccgtaccagggcgtgggcatccagctgttcgactccgagaccgagctgccgccggagaccaccgagaggccgaactacgagtcctactcccacaggctgtcccacatcggcctgatcatcggcaacaccctgagggccccggtgtactcctggacccacaggtccgccgacaggaccaacaccatcggcccgaacaggatcacccagatcccgctggtgaaggccctgaacctgcactccggcgtgaccggcgtgaggggcccgggcttcaccggcggcgacatcctgaggaggaccaacaccggcaccttcggcgacatcaggctgaacatcaacgtgccgctgtcccagaggtacagggtgaggatcaggtacgcctccaccaccgacctgcagttcttcaccaggatcaacggcaccaccgtgaacatcggcaacttctccaggaccatgaacaggggcgacaacctggagtacaggtccttcaggaccgccggcttctccaccccgttcaacttcctgaacgcccagtccaccttcaccctgggcgcccagtccttctccaaccaggaggtgtacatcgacagggtggagttcgtgccggccgaggtgaccttcgaggccgagtacgacctggagagggcccagaaggccgtgaacgccctgttcacctccaccaacccgaggaggctgaagaccgacgtgaccgactaccacatcgaccaggtgtccaacatggtggcctgcctgtccgac

SEQ ID NO: 58 - DNA, 2043 bp, synthetic construct
  source 1..2043: mol_type = other DNA
atgacctccaacaggaagaacgagaacgagatcatcaacgccctgtccatcccgaccgtgtccaacccgtccacccagatgaacctgtccccggacgccaggatcgaggactccctgtgcgtggccgaggtgaacaacatcgacccgttcgtgtccgcctccaccgtgcagaccggcatcaacatcgccggcaggatcctgggcgtgctgggcgtgccgttcgccggccagctggcctccttctactccttcctggtgggcgagctgtggccgtccggcagggacccgtgggagatcttcctggagcacgtggagcagctgatcaggcagcaggtgaccgagaacaccaggaacaccgccatcgccaggctggagggcctgggcaggggctacaggtcctaccagcaggccctggagacctggctggacaacaggaacgacgccaggtccaggtccatcatcctggagaggtacgtggccctggagctggacatcaccaccgccatcccgctgttcaggatcaggaaccaggaggtgccgctgctgatggtgtacgcccaggccgccaacctgcacctgctgctgctgagggacgcctccctgttcggctccgagtggggcatggcctcctccgacgtgaaccagtactaccaggagcagatcaggtacaccgaggagtactccaaccactgcgtgcagtggtacaacaccggcctgaacaacctgaggggcaccaacgccgagtcctggctgaggtacaaccagttcaggagggacctgaccctgggcgtgctggacctggtggccctgttcccgtcctacgacaccaggacctacccgatcaacaccgtgtcccagctgaccagggagatctacaccaacccggtgctggagaacttcgacggctccttcaggggctccgcccagggcatcgagggctccatcaggtccccgcacctgatggacatcctgaactccatcaccatctacaccgacgcccacaggggcgagtactactggtccggccaccagatcatggcctccccggtgggcttctccggcccggagttcaccttcccgctgtacggcaccatgggcaacgccgccccgcagcagaggatcgtggcccagctgggccagggcgtgtacaggaccctgtcctccaccctgtacaggaggccgttcaacatcggcatcaacaaccagcagctgtccgtgctggacggcaccgagttcgcctacggcacctcctccaacctgccgtccgccgtgtacaggaagtccggcaccgtggactccctggacgagatcccgccgcagaacaacaacgtgccgccgaggcagggcttctcccacaggctgtcccacgtgtccatgttcaggtccggcttctccaactcctccgtgtccatcatcagggccccgatgttctcctggatccacaggtccgccgagttcaacaacatcatcccgtcctcccagatcacccagatcccgctgaccaagtccaccaacctgggctccggcacctccgtggtgaagggcccgggcttcaccggcggcgacatcctgaggaggacctccccgggccagatctccaccctgagggtgaacatcaccgccccgctgtcccagaggtacagggtgaggatcaggtacgcctccaccaccaacctgcagttccacacctccatcgacggcaggccgatcaaccagggcaacttctccgccaccatgtcctccggctccaacctgcagtccggctccttcaggaccgtgggcttcaccaccccgttcaacttctccaacggctcctccgtgttcaccctgtccgcccacgtgttcaactccggcaacgaggtgtacatcgacaggatcgagttcgtgccggccgaggtgaccttcgaggccgagtacgacctggagagggcccagaaggccgtgaacgagctgttcacctcctccaaccagatcggcctgaagaccgacgtgaccgactaccacatcgaccaggtgtccaacctggtggagtgcctgtccgac

SEQ ID NO: 59 - DNA, 2043 bp, synthetic construct
  source 1..2043: mol_type = other DNA
atgacctccaacaggaagaacgagaacgagatcatcaacgccctgtccatcccgaccgtgtccaacccgtccacccagatgaacctgtccccggacgccaggatcgaggactccctgtgcgtggccgaggtgaacaacatcgacccgttcgtgtccgcctccaccgtgcagaccggcatcaacatcgccggcaggatcctgggcgtgctgggcgtgccgttcgccggccagctggcctccttctactccttcctggtgggcgagctgtggccgtccggcagggacccgtgggagatcttcctggagcacgtggagcagctgatcaggcagcaggtgaccgagaacaccaggaacaccgccatcgccaggctggagggcctgggcaggggctacaggtcctaccagcaggccctggagacctggctggacaacaggaacgacgccaggtccaggtccatcatcctggagaggtacgtggccctggagctggacatcaccaccgccatcccgctgttcaggatcaggaaccaggaggtgccgctgctgatggtgtacgcccaggccgccaacctgcacctgctgctgctgagggacgcctccctgttcggctccgagtggggcatggcctcctccgacgtgaaccagtactaccaggagcagatcaggtacaccgaggagtactccaaccactgcgtgcagtggtacaacaccggcctgaacaacctgaggggcaccaacgccgagtcctggctgaggtacaaccagttcaggagggacctgaccctgggcgtgctggacctggtggccctgttcccgtcctacgacaccaggacctacccgatcaacaccgtgtcccagctgaccagggagatctacaccaacccggtgctggagaacttcgacggctccttcaggggctccgcccagggcatcgagggctccatcaggtccccgcacctgatggacatcctgaactccatcaccatctacaccgacgcccacaggggcgagtactactggtccggccaccagatcatggcctccccggtgggcttctccggcccggagttcaccttcccgctgtacggcaccatgggcaacgccgccccgcagcagaggatcgtggcccagctgggccagggcgtgtacaggaccctgtcctccaccctgtacaggaggccgttcaacatcggcatcaacaaccagcagctgtccgtgctggacggcaccgagttcgcctacggcacctcctccaacctgccgtccgccgtgtacaggaagtccggcaccgtggactccctggacgagatcccgccgcagaacaacaacgtgccgccgaggcagggcttctcccacaggctgtcccacgtgtccatgttcaggtccggcttctccaactcctccgtgtccatcatcagggccccgatgttctcctggatccacaggtccgccgagttcaggaccaacaccatcggcccgaacaggatcacccagatcccgctggtgaaggccctgaacctgcactccggcgtgaccggcgtgaggggcccgggcttcaccggcggcgacatcctgaggaggaccaacaccggcaccttcggcgacatcaggctgaacatcaacgtgccgctgtcccagaggtacagggtgaggatcaggtacgcctccaccaccgacctgcagttcttcaccaggatcaacggcaccaccgtgaacatcggcaacttctccaggaccatgaacaggggcgacaacctggagtacaggtccttcaggaccgccggcttctccaccccgttcaacttcctgaacgcccagtccaccttcaccctgggcgcccagtccttctccaaccaggaggtgtacatcgacagggtggagttcgtgccggccgaggtgaccttcgaggccgagtacgacctggagagggcccagaaggccgtgaacgccctgttcacctccaccaacccgaggaggctgaagaccgacgtgaccgactaccacatcgaccaggtgtccaacatggtggcctgcctgtccgac

SEQ ID NO: 60 - DNA, 2070 bp, synthetic construct
  source 1..2070: mol_type = other DNA
atgacctccaacaggaagaacgagaacgagatcatcaacgccctgtccatcccgaccgtgtccaacccgtccacccagatgaacctgtccccggacgccaggatcgaggactccctgtgcgtggccgaggtgaacaacatcgacccgttcgtgtccgcctccaccgtgcagaccggcatcaacatcgccggcaggatcctgggcgtgctgggcgtgccgttcgccggccagctggcctccttctactccttcctggtgggcgagctgtggccgtccggcagggacccgtgggagatcttcctggagcacgtggagcagctgatcaggcagcaggtgaccgagaacaccaggaacaccgccatcgccaggctggagggcctgggcaggggctacaggtcctaccagcaggccctggagacctggctggacaacaggaacgacgccaggtccaggtccatcatcctggagaggtacgtggccctggagctggacatcaccaccgccatcccgctgttcaggatcaggaaccaggaggtgccgctgctgatggtgtacgcccaggccgccaacctgcacctgctgctgctgagggacgcctccctgttcggctccgagtggggcatggcctcctccgacgtgaaccagtactaccaggagcagatcaggtacaccgaggagtactccaaccactgcgtgcagtggtacaacaccggcctgaacaacctgaggggcaccaacgccgagtcctggctgaggtacaaccagttcaggagggacctgaccctgggcgtgctggacctggtggccctgttcccgtcctacgacaccaggacctacccgatcaacacctccgcccagctgaccagggagatctacaccgacccgatcggcaggaccaacgccccgtccggcttcgcctccaccaactggttcaacaacaacgccccgtccttctccgccatcgaggccgccatcatcaggccgccgcacctgctggacttcccggagcagctgaccatctactccgcctcctccaggtggtcctccacccagcacatgatctactgggtgggccacaggctgaacttcaggccgatcggcggcaccctgaacacctccacccagggcctgaccaacaacacctccatcaacccggtgaccctgcagttcacctccagggacgtgtacaggaccgagtcctacgccggcaccaacatcctgttcaccaccccggtgaacggcgtgccgtgggccaggttcaacttcatcaacccgcagaacatctacgagaggggcaccaccacctactcccagccgtaccagggcgtgggcatccagctgttcgactccgagaccgagctgccgccggagaccaccgagaggccgaactacgagtcctactcccacaggctgtcccacatcggcctgatcatcggcaacaccctgagggccccggtgtactcctggacccacaggtccgccgacaacaacatcatcccgtcctcccagatcacccagatcccgctgaccaagtccaccaacctgggctccggcacctccgtggtgaagggcccgggcttcaccggcggcgacatcctgaggaggacctccccgggccagatctccaccctgagggtgaacatcaccgccccgctgtcccagaggtacagggtgaggatcaggtacgcctccaccaccaacctgcagttccacacctccatcgacggcaggccgatcaaccagggcaacttctccgccaccatgtcctccggctccaacctgcagtccggctccttcaggaccgtgggcttcaccaccccgttcaacttctccaacggctcctccgtgttcaccctgtccgcccacgtgttcaactccggcaacgaggtgtacatcgacaggatcgagttcgtgccggccgaggtgaccttcgaggccgagtacgacctggagagggcccagaaggccgtgaacgagctgttcacctcctccaaccagatcggcctgaagaccgacgtgaccgactaccacatcgaccaggtgtccaacctggtggagtgcctgtccgac

SEQ ID NO: 61 - DNA, 1971 bp, synthetic construct
  source 1..1971: mol_type = other DNA
atggacaacaacccgaacatcaacgagtgcatcccgtacaactgcctgtccaacccggaggtggaggtgctgggcggcgagaggatcgagaccggctacaccccgatcgacatctccctgtccctgacccagttcctgctgtccgagttcgtgccgggcgccggcttcgtgctgggcctggtggacatcatctggggcatcttcggcccgtcccagtgggacgccttcctggtgcagatcgagcagctgatcaaccagaggatcgaggagttcgccaggaaccaggccatctccaggctggagggcctgtccaacctgtaccagatctacgccgagtccttcagggagtgggaggccgacccgaccaacccggccctgagggaggagatgaggatccagttcaacgacatgaactccgccctgaccaccgccatcccgctgttcgccgtgcagaactaccaggtgccgctgctgtccgtgtacgtgcaggccgccaacctgcacctgtccgtgctgagggacgtgtccgtgttcggccagaggtggggcttcgacgccgccaccatcaactccaggtacaacgacctgaccaggctgatcggcaactacaccgaccacgccgtgaggtggtacaacaccggcctggagagggtgtggggcccggactccagggactggatcaggtacaaccagttcaggagggagctgaccctgaccgtgctggacatcgtgtccctgttcccgaactacgactccaggacctacccgatcaggaccgtgtcccagctgaccagggagatctacaccaacccggtgctggagaacttcgacggctccttcaggggctccgcccagggcatcgagggctccatcaggtccccgcacctgatggacatcctgaactccatcaccatctacaccgacgcccacaggggcgagtactactggtccggccaccagatcatggcctccccggtgggcttctccggcccggagttcaccttcccgctgtacggcaccatgggcaacgccgccccgcagcagaggatcgtggcccagctgggccagggcgtgtacaggaccctgtcctccaccctgtacaggaggccgttcaacatcggcatcaacaaccagcagctgtccgtgctggacggcaccgagttcgcctacggcacctcctccaacctgccgtccgccgtgtacaggaagtccggcaccgtggactccctggacgagatcccgccgcagaacaacaacgtgccgccgaggcagggcttctcccacaggctgtcccacgtgtccatgttcaggtccggcttctccaactcctccgtgtccatcatcagggccccgatgttctcctggatccacaggtccgccgagttcaccaacaccaacaccatcaacccggacatcatcacccagatcccgctggtgaagggcttcaggctgggcggcggcacctccgtgatcaagggcccgggcttcaccggcggcgacatcctgaggaggaacaccatcggcgagttcgtgtccctgcaggtgaacatcaactccccgatcacccagaggtacaggctgaggttcaggtacgcctcctccagggacgccaggatcaccgtggccatcggcggccagatcagggtggacatgaccctggagaagaccatggagatcggcgagtccctgacctccaggaccttctcctacaccaacttctccaacccgttctccttcagggccaacccggacatcatcaggatcgccgaggagctgccgatcaggggcggcgagctgtacatcgacaagatcgagctgatcctggccgacgccaccttcgaggaggagtacgacctggagagggcccagaaggccgtgaacgccctgttcacctccaccaaccagctgggcctgaagaccgacgtgaccgactaccacatcgaccaggtgtccaacctggtggagtgcctg

SEQ ID NO: 62 - DNA, 1830 bp, synthetic construct
  source 1..1830: mol_type = other DNA
atggagatcgtgaacaaccagaaccagtgcgtgccgtacaactgcctgaacaacccggagaacgagatcctggacatcgagaggtccaactccaccgtggccaccaacatcgccctggagatctccaggctgctggcctccgccaccccgatcggcggcatcctgctgggcctgttcgacgccatctggggctccatcggcccgtcccagtgggacctgttcctggagcagatcgagctgctgatcgaccagaagatcgaggagttcgccaggaaccaggccatctccaggctggagggcatctcctccctgtacggcatctacaccgaggccttcagggagtgggaggccgacccgaccaacccggccctgaaggaggagatgaggacccagttcaacgacatgaactccatcctggtgaccgccatcccgctgttctccgtgcagaactaccaggtgccgttcctgtccgtgtacgtgcaggccgccaacctgcacctgtccgtgctgagggacgtgtccgtgttcggccaggcctggggcttcgacatcgccaccatcaactccaggtacaacgacctgaccaggctgatcccgatctacaccgactacgccgtgaggtggtacaacaccggcctggacaggctgccgaggaccggcggcctgaggaactgggccaggttcaaccagttcaggagggagctgaccatctccgtgctggacatcatctccttcttcaggaactacgactccaggctgtacccgatcccgacctcctcccagctgaccagggaggtgtacaccgacccggtgatcaacatcaccgactacagggtgggcccgtccttcgagaacatcgagaactccgccatcaggtccccgcacctgatggacttcctgaacaacctgaccatcgacaccgacctgatcaggggcgtgcactactgggccggccacagggtgacctcccacttcaccggctcctcccaggtgatcaccaccccgcagtacggcatcaccgccaacgccgagccgaggaggaccatcgccccgtccaccttcccgggcctgaacctgttctacaggaccctgtccaacccgttcttcaggaggtccgagaacatcaccccgaccctgggcatcaacgtggtgcagggcgtgggcttcatccagccgaacaacgccgaggtgctgtacaggtccaggggcaccgtggactccctgaacgagctgccgatcgacggcgagaactccctggtgggctactcccacaggctgtcccacgtgaccctgaccaggtccctgtacaacaccaacatcacctccctgccgaccttcgtgtggacccaccactccgccaacaacatcatcccgtcctcccagatcacccagatcccgctgaccaagtccaccaacctgggctccggcacctccgtggtgaagggcccgggcttcaccggcggcgacatcctgaggaggacctccccgggccagatctccaccctgagggtgaacatcaccgccccgctgtcccagaggtacagggtgaggatcaggtacgcctccaccaccaacctgcagttccacacctccatcgacggcaggccgatcaaccagggcaacttctccgccaccatgtcctccggctccaacctgcagtccggctccttcaggaccgtgggcttcaccaccccgttcaacttctccaacggctcctccgtgttcaccctgtccgcccacgtgttcaactccggcaacgaggtgtacatcgacaggatcgagttcgtgccggccgaggtgaccttcgaggccgagtacgacctggagagg

SEQ ID NO: 63 - DNA, 1968 bp, synthetic construct
  source 1..1968: mol_type = other DNA
atggagatcgtgaacaaccagaaccagtgcgtgccgtacaactgcctgaacaacccggagaacgagatcctggacatcgagaggtccaactccaccgtggccaccaacatcgccctggagatctccaggctgctggcctccgccaccccgatcggcggcatcctgctgggcctgttcgacgccatctggggctccatcggcccgtcccagtgggacctgttcctggagcagatcgagctgctgatcgaccagaagatcgaggagttcgccaggaaccaggccatctccaggctggagggcatctcctccctgtacggcatctacaccgaggccttcagggagtgggaggccgacccgaccaacccggccctgaaggaggagatgaggacccagttcaacgacatgaactccatcctggtgaccgccatcccgctgttctccgtgcagaactaccaggtgccgttcctgtccgtgtacgtgcaggccgccaacctgcacctgtccgtgctgagggacgtgtccgtgttcggccaggcctggggcttcgacatcgccaccatcaactccaggtacaacgacctgaccaggctgatcccgatctacaccgactacgccgtgaggtggtacaacaccggcctggacaggctgccgaggaccggcggcctgaggaactgggccaggttcaaccagttcaggagggagctgaccatctccgtgctggacatcatctccttcttcaggaactacgactccaggctgtacccgatcccgaccgtgtcccagctgaccagggagatctacaccaacccggtgctggagaacttcgacggctccttcaggggctccgcccagggcatcgagggctccatcaggtccccgcacctgatggacatcctgaactccatcaccatctacaccgacgcccacaggggcgagtactactggtccggccaccagatcatggcctccccggtgggcttctccggcccggagttcaccttcccgctgtacggcaccatgggcaacgccgccccgcagcagaggatcgtggcccagctgggccagggcgtgtacaggaccctgtcctccaccctgtacaggaggccgttcaacatcggcatcaacaaccagcagctgtccgtgctggacggcaccgagttcgcctacggcacctcctccaacctgccgtccgccgtgtacaggaagtccggcaccgtggactccctggacgagatcccgccgcagaacaacaacgtgccgccgaggcagggcttctcccacaggctgtcccacgtgtccatgttcaggtccggcttctccaactcctccgtgtccatcatcagggccccgatgttctcctggatccacaggtccgccgagttcaccaacaccaacaccatcaacccggacatcatcacccagatcccgctggtgaagggcttcaggctgggcggcggcacctccgtgatcaagggcccgggcttcaccggcggcgacatcctgaggaggaacaccatcggcgagttcgtgtccctgcaggtgaacatcaactccccgatcacccagaggtacaggctgaggttcaggtacgcctcctccagggacgccaggatcaccgtggccatcggcggccagatcagggtggacatgaccctggagaagaccatggagatcggcgagtccctgacctccaggaccttctcctacaccaacttctccaacccgttctccttcagggccaacccggacatcatcaggatcgccgaggagctgccgatcaggggcggcgagctgtacatcgacaagatcgagctgatcctggccgacgccaccttcgaggaggagtacgacctggagagggcccagaaggccgtgaacgccctgttcacctccaccaaccagctgggcctgaagaccgacgtgaccgactaccacatcgaccaggtgtccaacctggtggagtgcctg

SEQ ID NO: 64 - DNA, 1854 bp, synthetic construct
  source 1..1854: mol_type = other DNA
atggagatcgtgaacaaccagaaccagtgcgtgccgtacaactgcctgaacaacccggagaacgagatcctggacatcgagaggtccaactccaccgtggccaccaacatcgccctggagatctccaggctgctggcctccgccaccccgatcggcggcatcctgctgggcctgttcgacgccatctggggctccatcggcccgtcccagtgggacctgttcctggagcagatcgagctgctgatcgaccagaagatcgaggagttcgccaggaaccaggccatctccaggctggagggcatctcctccctgtacggcatctacaccgaggccttcagggagtgggaggccgacccgaccaacccggccctgaaggaggagatgaggacccagttcaacgacatgaactccatcctggtgaccgccatcccgctgttctccgtgcagaactaccaggtgccgttcctgtccgtgtacgtgcaggccgccaacctgcacctgtccgtgctgagggacgtgtccgtgttcggccaggcctggggcttcgacatcgccaccatcaactccaggtacaacgacctgaccaggctgatcccgatctacaccgactacgccgtgaggtggtacaacaccggcctggacaggctgccgaggaccggcggcctgaggaactgggccaggttcaaccagttcaggagggagctgaccatctccgtgctggacatcatctccttcttcaggaactacgactccaggctgtacccgatcccgaccgtgtcccagctgaccagggagatctacaccaacccggtgctggagaacttcgacggctccttcaggggctccgcccagggcatcgagggctccatcaggtccccgcacctgatggacatcctgaactccatcaccatctacaccgacgcccacaggggcgagtactactggtccggccaccagatcatggcctccccggtgggcttctccggcccggagttcaccttcccgctgtacggcaccatgggcaacgccgccccgcagcagaggatcgtggcccagctgggccagggcgtgtacaggaccctgtcctccaccctgtacaggaggccgttcaacatcggcatcaacaaccagcagctgtccgtgctggacggcaccgagttcgcctacggcacctcctccaacctgccgtccgccgtgtacaggaagtccggcaccgtggactccctggacgagatcccgccgcagaacaacaacgtgccgccgaggcagggcttctcccacaggctgtcccacgtgtccatgttcaggtccggcttctccaactcctccgtgtccatcatcagggccccgatgttctcctggatccacaggtccgccgagttcaacaacatcatcccgtcctcccagatcacccagatcccgctgaccaagtccaccaacctgggctccggcacctccgtggtgaagggcccgggcttcaccggcggcgacatcctgaggaggacctccccgggccagatctccaccctgagggtgaacatcaccgccccgctgtcccagaggtacagggtgaggatcaggtacgcctccaccaccaacctgcagttccacacctccatcgacggcaggccgatcaaccagggcaacttctccgccaccatgtcctccggctccaacctgcagtccggctccttcaggaccgtgggcttcaccaccccgttcaacttctccaacggctcctccgtgttcaccctgtccgcccacgtgttcaactccggcaacgaggtgtacatcgacaggatcgagttcgtgccggccgaggtgaccttcgaggccgagtacgacctggagagg

SEQ ID NO: 65 - DNA, 1947 bp, synthetic construct
  source 1..1947: mol_type = other DNA
atggacaacaacccgaacatcaacgagtgcatcccgtacaactgcctgtccaacccggaggtggaggtgctgggcggcgagaggatcgagaccggctacaccccgatcgacatctccctgtccctgacccagttcctgctgtccgagttcgtgccgggcgccggcttcgtgctgggcctggtggacatcatctggggcatcttcggcccgtcccagtgggacgccttcctggtgcagatcgagcagctgatcaaccagaggatcgaggagttcgccaggaaccaggccatctccaggctggagggcctgtccaacctgtaccagatctacgccgagtccttcagggagtgggaggccgacccgaccaacccggccctgagggaggagatgaggatccagttcaacgacatgaactccgccctgaccaccgccatcccgctgttcgccgtgcagaactaccaggtgccgctgctgtccgtgtacgtgcaggccgccaacctgcacctgtccgtgctgagggacgtgtccgtgttcggccagaggtggggcttcgacgccgccaccatcaactccaggtacaacgacctgaccaggctgatcggcaactacaccgaccacgccgtgaggtggtacaacaccggcctggagagggtgtggggcccggactccagggactggatcaggtacaaccagttcaggagggagctgaccctgaccgtgctggacatcgtgtccctgttcccgaactacgactccaggacctacccgatcaggacctcctcccagctgaccagggaggtgtacaccgacccggtgatcaacatcaccgactacagggtgggcccgtccttcgagaacatcgagaactccgccatcaggtccccgcacctgatggacttcctgaacaacctgaccatcgacaccgacctgatcaggggcgtgcactactgggccggccacagggtgacctcccacttcaccggctcctcccaggtgatcaccaccccgcagtacggcatcaccgccaacgccgagccgaggaggaccatcgccccgtccaccttcccgggcctgaacctgttctacaggaccctgtccaacccgttcttcaggaggtccgagaacatcaccccgaccctgggcatcaacgtggtgcagggcgtgggcttcatccagccgaacaacgccgaggtgctgtacaggtccaggggcaccgtggactccctgaacgagctgccgatcgacggcgagaactccctggtgggctactcccacaggctgtcccacgtgaccctgaccaggtccctgtacaacaccaacatcacctccctgccgaccttcgtgtggacccaccactccgccaccaacaccaacaccatcaacccggacatcatcacccagatcccgctggtgaagggcttcaggctgggcggcggcacctccgtgatcaagggcccgggcttcaccggcggcgacatcctgaggaggaacaccatcggcgagttcgtgtccctgcaggtgaacatcaactccccgatcacccagaggtacaggctgaggttcaggtacgcctcctccagggacgccaggatcaccgtggccatcggcggccagatcagggtggacatgaccctggagaagaccatggagatcggcgagtccctgacctccaggaccttctcctacaccaacttctccaacccgttctccttcagggccaacccggacatcatcaggatcgccgaggagctgccgatcaggggcggcgagctgtacatcgacaagatcgagctgatcctggccgacgccaccttcgaggaggagtacgacctggagagggcccagaaggccgtgaacgccctgttcacctccaccaaccagctgggcctgaagaccgacgtgaccgactaccacatcgaccaggtgtccaacctggtggagtgcctg

SEQ ID NO: 66 - DNA, 1833 bp, synthetic construct
  source 1..1833: mol_type = other DNA
atggacaacaacccgaacatcaacgagtgcatcccgtacaactgcctgtccaacccggaggtggaggtgctgggcggcgagaggatcgagaccggctacaccccgatcgacatctccctgtccctgacccagttcctgctgtccgagttcgtgccgggcgccggcttcgtgctgggcctggtggacatcatctggggcatcttcggcccgtcccagtgggacgccttcctggtgcagatcgagcagctgatcaaccagaggatcgaggagttcgccaggaaccaggccatctccaggctggagggcctgtccaacctgtaccagatctacgccgagtccttcagggagtgggaggccgacccgaccaacccggccctgagggaggagatgaggatccagttcaacgacatgaactccgccctgaccaccgccatcccgctgttcgccgtgcagaactaccaggtgccgctgctgtccgtgtacgtgcaggccgccaacctgcacctgtccgtgctgagggacgtgtccgtgttcggccagaggtggggcttcgacgccgccaccatcaactccaggtacaacgacctgaccaggctgatcggcaactacaccgaccacgccgtgaggtggtacaacaccggcctggagagggtgtggggcccggactccagggactggatcaggtacaaccagttcaggagggagctgaccctgaccgtgctggacatcgtgtccctgttcccgaactacgactccaggacctacccgatcaggacctcctcccagctgaccagggaggtgtacaccgacccggtgatcaacatcaccgactacagggtgggcccgtccttcgagaacatcgagaactccgccatcaggtccccgcacctgatggacttcctgaacaacctgaccatcgacaccgacctgatcaggggcgtgcactactgggccggccacagggtgacctcccacttcaccggctcctcccaggtgatcaccaccccgcagtacggcatcaccgccaacgccgagccgaggaggaccatcgccccgtccaccttcccgggcctgaacctgttctacaggaccctgtccaacccgttcttcaggaggtccgagaacatcaccccgaccctgggcatcaacgtggtgcagggcgtgggcttcatccagccgaacaacgccgaggtgctgtacaggtccaggggcaccgtggactccctgaacgagctgccgatcgacggcgagaactccctggtgggctactcccacaggctgtcccacgtgaccctgaccaggtccctgtacaacaccaacatcacctccctgccgaccttcgtgtggacccaccactccgccaacaacatcatcccgtcctcccagatcacccagatcccgctgaccaagtccaccaacctgggctccggcacctccgtggtgaagggcccgggcttcaccggcggcgacatcctgaggaggacctccccgggccagatctccaccctgagggtgaacatcaccgccccgctgtcccagaggtacagggtgaggatcaggtacgcctccaccaccaacctgcagttccacacctccatcgacggcaggccgatcaaccagggcaacttctccgccaccatgtcctccggctccaacctgcagtccggctccttcaggaccgtgggcttcaccaccccgttcaacttctccaacggctcctccgtgttcaccctgtccgcccacgtgttcaactccggcaacgaggtgtacatcgacaggatcgagttcgtgccggccgaggtgaccttcgaggccgagtacgacctggagagg

SEQ ID NO: 67 - DNA, 1944 bp, synthetic construct
  source 1..1944: mol_type = other DNA
atggagatcgtgaacaaccagaaccagtgcgtgccgtacaactgcctgaacaacccggagaacgagatcctggacatcgagaggtccaactccaccgtggccaccaacatcgccctggagatctccaggctgctggcctccgccaccccgatcggcggcatcctgctgggcctgttcgacgccatctggggctccatcggcccgtcccagtgggacctgttcctggagcagatcgagctgctgatcgaccagaagatcgaggagttcgccaggaaccaggccatctccaggctggagggcatctcctccctgtacggcatctacaccgaggccttcagggagtgggaggccgacccgaccaacccggccctgaaggaggagatgaggacccagttcaacgacatgaactccatcctggtgaccgccatcccgctgttctccgtgcagaactaccaggtgccgttcctgtccgtgtacgtgcaggccgccaacctgcacctgtccgtgctgagggacgtgtccgtgttcggccaggcctggggcttcgacatcgccaccatcaactccaggtacaacgacctgaccaggctgatcccgatctacaccgactacgccgtgaggtggtacaacaccggcctggacaggctgccgaggaccggcggcctgaggaactgggccaggttcaaccagttcaggagggagctgaccatctccgtgctggacatcatctccttcttcaggaactacgactccaggctgtacccgatcccgacctcctcccagctgaccagggaggtgtacaccgacccggtgatcaacatcaccgactacagggtgggcccgtccttcgagaacatcgagaactccgccatcaggtccccgcacctgatggacttcctgaacaacctgaccatcgacaccgacctgatcaggggcgtgcactactgggccggccacagggtgacctcccacttcaccggctcctcccaggtgatcaccaccccgcagtacggcatcaccgccaacgccgagccgaggaggaccatcgccccgtccaccttcccgggcctgaacctgttctacaggaccctgtccaacccgttcttcaggaggtccgagaacatcaccccgaccctgggcatcaacgtggtgcagggcgtgggcttcatccagccgaacaacgccgaggtgctgtacaggtccaggggcaccgtggactccctgaacgagctgccgatcgacggcgagaactccctggtgggctactcccacaggctgtcccacgtgaccctgaccaggtccctgtacaacaccaacatcacctccctgccgaccttcgtgtggacccaccactccgccaggaccaacaccatcggcccgaacaggatcacccagatcccgctggtgaaggccctgaacctgcactccggcgtgaccggcgtgaggggcccgggcttcaccggcggcgacatcctgaggaggaccaacaccggcaccttcggcgacatcaggctgaacatcaacgtgccgctgtcccagaggtacagggtgaggatcaggtacgcctccaccaccgacctgcagttcttcaccaggatcaacggcaccaccgtgaacatcggcaacttctccaggaccatgaacaggggcgacaacctggagtacaggtccttcaggaccgccggcttctccaccccgttcaacttcctgaacgcccagtccaccttcaccctgggcgcccagtccttctccaaccaggaggtgtacatcgacagggtggagttcgtgccggccgaggtgaccttcgaggccgagtacgacctggagagggcccagaaggccgtgaacgccctgttcacctccaccaacccgaggaggctgaagaccgacgtgaccgactaccacatcgaccaggtgtccaacatggtggcctgcctgtccgac

SEQ ID NO: 68 - DNA, 1995 bp, synthetic construct
  source 1..1995: mol_type = other DNA
atggagatcgtgaacaaccagaaccagtgcgtgccgtacaactgcctgaacaacccggagaacgagatcctggacatcgagaggtccaactccaccgtggccaccaacatcgccctggagatctccaggctgctggcctccgccaccccgatcggcggcatcctgctgggcctgttcgacgccatctggggctccatcggcccgtcccagtgggacctgttcctggagcagatcgagctgctgatcgaccagaagatcgaggagttcgccaggaaccaggccatctccaggctggagggcatctcctccctgtacggcatctacaccgaggccttcagggagtgggaggccgacccgaccaacccggccctgaaggaggagatgaggacccagttcaacgacatgaactccatcctggtgaccgccatcccgctgttctccgtgcagaactaccaggtgccgttcctgtccgtgtacgtgcaggccgccaacctgcacctgtccgtgctgagggacgtgtccgtgttcggccaggcctggggcttcgacatcgccaccatcaactccaggtacaacgacctgaccaggctgatcccgatctacaccgactacgccgtgaggtggtacaacaccggcctggacaggctgccgaggaccggcggcctgaggaactgggccaggttcaaccagttcaggagggagctgaccatctccgtgctggacatcatctccttcttcaggaactacgactccaggctgtacccgatcccgacctccgcccagctgaccagggagatctacaccgacccgatcggcaggaccaacgccccgtccggcttcgcctccaccaactggttcaacaacaacgccccgtccttctccgccatcgaggccgccatcatcaggccgccgcacctgctggacttcccggagcagctgaccatctactccgcctcctccaggtggtcctccacccagcacatgatctactgggtgggccacaggctgaacttcaggccgatcggcggcaccctgaacacctccacccagggcctgaccaacaacacctccatcaacccggtgaccctgcagttcacctccagggacgtgtacaggaccgagtcctacgccggcaccaacatcctgttcaccaccccggtgaacggcgtgccgtgggccaggttcaacttcatcaacccgcagaacatctacgagaggggcaccaccacctactcccagccgtaccagggcgtgggcatccagctgttcgactccgagaccgagctgccgccggagaccaccgagaggccgaactacgagtcctactcccacaggctgtcccacatcggcctgatcatcggcaacaccctgagggccccggtgtactcctggacccacaggtccgccgacaccaacaccaacaccatcaacccggacatcatcacccagatcccgctggtgaagggcttcaggctgggcggcggcacctccgtgatcaagggcccgggcttcaccggcggcgacatcctgaggaggaacaccatcggcgagttcgtgtccctgcaggtgaacatcaactccccgatcacccagaggtacaggctgaggttcaggtacgcctcctccagggacgccaggatcaccgtggccatcggcggccagatcagggtggacatgaccctggagaagaccatggagatcggcgagtccctgacctccaggaccttctcctacaccaacttctccaacccgttctccttcagggccaacccggacatcatcaggatcgccgaggagctgccgatcaggggcggcgagctgtacatcgacaagatcgagctgatcctggccgacgccaccttcgaggaggagtacgacctggagagggcccagaaggccgtgaacgccctgttcacctccaccaaccagctgggcctgaagaccgacgtgaccgactaccacatcgaccaggtgtccaacctggtggagtgcctg

SEQ ID NO: 69 - DNA, 1995 bp, synthetic construct
  source 1..1995: mol_type = other DNA
atggagatcgtgaacaaccagaaccagtgcgtgccgtacaactgcctgaacaacccggagaacgagatcctggacatcgagaggtccaactccaccgtggccaccaacatcgccctggagatctccaggctgctggcctccgccaccccgatcggcggcatcctgctgggcctgttcgacgccatctggggctccatcggcccgtcccagtgggacctgttcctggagcagatcgagctgctgatcgaccagaagatcgaggagttcgccaggaaccaggccatctccaggctggagggcatctcctccctgtacggcatctacaccgaggccttcagggagtgggaggccgacccgaccaacccggccctgaaggaggagatgaggacccagttcaacgacatgaactccatcctggtgaccgccatcccgctgttctccgtgcagaactaccaggtgccgttcctgtccgtgtacgtgcaggccgccaacctgcacctgtccgtgctgagggacgtgtccgtgttcggccaggcctggggcttcgacatcgccaccatcaactccaggtacaacgacctgaccaggctgatcccgatctacaccgactacgccgtgaggtggtacaacaccggcctggacaggctgccgaggaccggcggcctgaggaactgggccaggttcaaccagttcaggagggagctgaccatctccgtgctggacatcatctccttcttcaggaactacgactccaggctgtacccgatcccgacctccgcccagctgaccagggagatctacaccgacccgatcggcaggaccaacgccccgtccggcttcgcctccaccaactggttcaacaacaacgccccgtccttctccgccatcgaggccgccatcatcaggccgccgcacctgctggacttcccggagcagctgaccatctactccgcctcctccaggtggtcctccacccagcacatgatctactgggtgggccacaggctgaacttcaggccgatcggcggcaccctgaacacctccacccagggcctgaccaacaacacctccatcaacccggtgaccctgcagttcacctccagggacgtgtacaggaccgagtcctacgccggcaccaacatcctgttcaccaccccggtgaacggcgtgccgtgggccaggttcaacttcatcaacccgcagaacatctacgagaggggcaccaccacctactcccagccgtaccagggcgtgggcatccagctgttcgactccgagaccgagctgccgccggagaccaccgagaggccgaactacgagtcctactcccacaggctgtcccacatcggcctgatcatcggcaacaccctgagggccccggtgtactcctggacccacaggtccgccgacaggaccaacaccatcggcccgaacaggatcacccagatcccgctggtgaaggccctgaacctgcactccggcgtgaccggcgtgaggggcccgggcttcaccggcggcgacatcctgaggaggaccaacaccggcaccttcggcgacatcaggctgaacatcaacgtgccgctgtcccagaggtacagggtgaggatcaggtacgcctccaccaccgacctgcagttcttcaccaggatcaacggcaccaccgtgaacatcggcaacttctccaggaccatgaacaggggcgacaacctggagtacaggtccttcaggaccgccggcttctccaccccgttcaacttcctgaacgcccagtccaccttcaccctgggcgcccagtccttctccaaccaggaggtgtacatcgacagggtggagttcgtgccggccgaggtgaccttcgaggccgagtacgacctggagagggcccagaaggccgtgaacgccctgttcacctccaccaacccgaggaggctgaagaccgacgtgaccgactaccacatcgaccaggtgtccaacatggtggcctgcctgtccgac

SEQ ID NO: 70 - DNA, 2019 bp, synthetic construct
  source 1..2019: mol_type = other DNA
atgacctccaacaggaagaacgagaacgagatcatcaacgccctgtccatcccgaccgtgtccaacccgtccacccagatgaacctgtccccggacgccaggatcgaggactccctgtgcgtggccgaggtgaacaacatcgacccgttcgtgtccgcctccaccgtgcagaccggcatcaacatcgccggcaggatcctgggcgtgctgggcgtgccgttcgccggccagctggcctccttctactccttcctggtgggcgagctgtggccgtccggcagggacccgtgggagatcttcctggagcacgtggagcagctgatcaggcagcaggtgaccgagaacaccaggaacaccgccatcgccaggctggagggcctgggcaggggctacaggtcctaccagcaggccctggagacctggctggacaacaggaacgacgccaggtccaggtccatcatcctggagaggtacgtggccctggagctggacatcaccaccgccatcccgctgttcaggatcaggaaccaggaggtgccgctgctgatggtgtacgcccaggccgccaacctgcacctgctgctgctgagggacgcctccctgttcggctccgagtggggcatggcctcctccgacgtgaaccagtactaccaggagcagatcaggtacaccgaggagtactccaaccactgcgtgcagtggtacaacaccggcctgaacaacctgaggggcaccaacgccgagtcctggctgaggtacaaccagttcaggagggacctgaccctgggcgtgctggacctggtggccctgttcccgtcctacgacaccaggacctacccgatcaacacctcctcccagctgaccagggaggtgtacaccgacccggtgatcaacatcaccgactacagggtgggcccgtccttcgagaacatcgagaactccgccatcaggtccccgcacctgatggacttcctgaacaacctgaccatcgacaccgacctgatcaggggcgtgcactactgggccggccacagggtgacctcccacttcaccggctcctcccaggtgatcaccaccccgcagtacggcatcaccgccaacgccgagccgaggaggaccatcgccccgtccaccttcccgggcctgaacctgttctacaggaccctgtccaacccgttcttcaggaggtccgagaacatcaccccgaccctgggcatcaacgtggtgcagggcgtgggcttcatccagccgaacaacgccgaggtgctgtacaggtccaggggcaccgtggactccctgaacgagctgccgatcgacggcgagaactccctggtgggctactcccacaggctgtcccacgtgaccctgaccaggtccctgtacaacaccaacatcacctccctgccgaccttcgtgtggacccaccactccgccaccaacaccaacaccatcaacccggacatcatcacccagatcccgctggtgaagggcttcaggctgggcggcggcacctccgtgatcaagggcccgggcttcaccggcggcgacatcctgaggaggaacaccatcggcgagttcgtgtccctgcaggtgaacatcaactccccgatcacccagaggtacaggctgaggttcaggtacgcctcctccagggacgccaggatcaccgtggccatcggcggccagatcagggtggacatgaccctggagaagaccatggagatcggcgagtccctgacctccaggaccttctcctacaccaacttctccaacccgttctccttcagggccaacccggacatcatcaggatcgccgaggagctgccgatcaggggcggcgagctgtacatcgacaagatcgagctgatcctggccgacgccaccttcgaggaggagtacgacctggagagggcccagaaggccgtgaacgccctgttcacctccaccaaccagctgggcctgaagaccgacgtgaccgactaccacatcgaccaggtgtccaacctggtggagtgcctg

SEQ ID NO: 71 - DNA, 2019 bp, synthetic construct
  source 1..2019: mol_type = other DNA
atgacctccaacaggaagaacgagaacgagatcatcaacgccctgtccatcccgaccgtgtccaacccgtccacccagatgaacctgtccccggacgccaggatcgaggactccctgtgcgtggccgaggtgaacaacatcgacccgttcgtgtccgcctccaccgtgcagaccggcatcaacatcgccggcaggatcctgggcgtgctgggcgtgccgttcgccggccagctggcctccttctactccttcctggtgggcgagctgtggccgtccggcagggacccgtgggagatcttcctggagcacgtggagcagctgatcaggcagcaggtgaccgagaacaccaggaacaccgccatcgccaggctggagggcctgggcaggggctacaggtcctaccagcaggccctggagacctggctggacaacaggaacgacgccaggtccaggtccatcatcctggagaggtacgtggccctggagctggacatcaccaccgccatcccgctgttcaggatcaggaaccaggaggtgccgctgctgatggtgtacgcccaggccgccaacctgcacctgctgctgctgagggacgcctccctgttcggctccgagtggggcatggcctcctccgacgtgaaccagtactaccaggagcagatcaggtacaccgaggagtactccaaccactgcgtgcagtggtacaacaccggcctgaacaacctgaggggcaccaacgccgagtcctggctgaggtacaaccagttcaggagggacctgaccctgggcgtgctggacctggtggccctgttcccgtcctacgacaccaggacctacccgatcaacacctcctcccagctgaccagggaggtgtacaccgacccggtgatcaacatcaccgactacagggtgggcccgtccttcgagaacatcgagaactccgccatcaggtccccgcacctgatggacttcctgaacaacctgaccatcgacaccgacctgatcaggggcgtgcactactgggccggccacagggtgacctcccacttcaccggctcctcccaggtgatcaccaccccgcagtacggcatcaccgccaacgccgagccgaggaggaccatcgccccgtccaccttcccgggcctgaacctgttctacaggaccctgtccaacccgttcttcaggaggtccgagaacatcaccccgaccctgggcatcaacgtggtgcagggcgtgggcttcatccagccgaacaacgccgaggtgctgtacaggtccaggggcaccgtggactccctgaacgagctgccgatcgacggcgagaactccctggtgggctactcccacaggctgtcccacgtgaccctgaccaggtccctgtacaacaccaacatcacctccctgccgaccttcgtgtggacccaccactccgccaggaccaacaccatcggcccgaacaggatcacccagatcccgctggtgaaggccctgaacctgcactccggcgtgaccggcgtgaggggcccgggcttcaccggcggcgacatcctgaggaggaccaacaccggcaccttcggcgacatcaggctgaacatcaacgtgccgctgtcccagaggtacagggtgaggatcaggtacgcctccaccaccgacctgcagttcttcaccaggatcaacggcaccaccgtgaacatcggcaacttctccaggaccatgaacaggggcgacaacctggagtacaggtccttcaggaccgccggcttctccaccccgttcaacttcctgaacgcccagtccaccttcaccctgggcgcccagtccttctccaaccaggaggtgtacatcgacagggtggagttcgtgccggccgaggtgaccttcgaggccgagtacgacctggagagggcccagaaggccgtgaacgccctgttcacctccaccaacccgaggaggctgaagaccgacgtgaccgactaccacatcgaccaggtgtccaacatggtggcctgcctgtccgac

SEQ ID NO: 72 - DNA, 2073 bp, synthetic construct
  source 1..2073: mol_type = other DNA
atgacctccaacaggaagaacgagaacgagatcatcaacgccctgtccatcccgaccgtgtccaacccgtccacccagatgaacctgtccccggacgccaggatcgaggactccctgtgcgtggccgaggtgaacaacatcgacccgttcgtgtccgcctccaccgtgcagaccggcatcaacatcgccggcaggatcctgggcgtgctgggcgtgccgttcgccggccagctggcctccttctactccttcctggtgggcgagctgtggccgtccggcagggacccgtgggagatcttcctggagcacgtggagcagctgatcaggcagcaggtgaccgagaacaccaggaacaccgccatcgccaggctggagggcctgggcaggggctacaggtcctaccagcaggccctggagacctggctggacaacaggaacgacgccaggtccaggtccatcatcctggagaggtacgtggccctggagctggacatcaccaccgccatcccgctgttcaggatcaggaaccaggaggtgccgctgctgatggtgtacgcccaggccgccaacctgcacctgctgctgctgagggacgcctccctgttcggctccgagtggggcatggcctcctccgacgtgaaccagtactaccaggagcagatcaggtacaccgaggagtactccaaccactgcgtgcagtggtacaacaccggcctgaacaacctgaggggcaccaacgccgagtcctggctgaggtacaaccagttcaggagggacctgaccctgggcgtgctggacctggtggccctgttcccgtcctacgacaccaggacctacccgatcaacacctccgcccagctgaccagggagatctacaccgacccgatcggcaggaccaacgccccgtccggcttcgcctccaccaactggttcaacaacaacgccccgtccttctccgccatcgaggccgccatcatcaggccgccgcacctgctggacttcccggagcagctgaccatctactccgcctcctccaggtggtcctccacccagcacatgatctactgggtgggccacaggctgaacttcaggccgatcggcggcaccctgaacacctccacccagggcctgaccaacaacacctccatcaacccggtgaccctgcagttcacctccagggacgtgtacaggaccgagtcctacgccggcaccaacatcctgttcaccaccccggtgaacggcgtgccgtgggccaggttcaacttcatcaacccgcagaacatctacgagaggggcaccaccacctactcccagccgtaccagggcgtgggcatccagctgttcgactccgagaccgagctgccgccggagaccaccgagaggccgaactacgagtcctactcccacaggctgtcccacatcggcctgatcatcggcaacaccctgagggccccggtgtactcctggacccacaggtccgccgacaccaacaccaacaccatcaacccggacatcatcacccagatcccgctggtgaagggcttcaggctgggcggcggcacctccgtgatcaagggcccgggcttcaccggcggcgacatcctgaggaggaacaccatcggcgagttcgtgtccctgcaggtgaacatcaactccccgatcacccagaggtacaggctgaggttcaggtacgcctcctccagggacgccaggatcaccgtggccatcggcggccagatcagggtggacatgaccctggagaagaccatggagatcggcgagtccctgacctccaggaccttctcctacaccaacttctccaacccgttctccttcagggccaacccggacatcatcaggatcgccgaggagctgccgatcaggggcggcgagctgtacatcgacaagatcgagctgatcctggccgacgccaccttcgaggaggagtacgacctggagagggcccagaaggccgtgaacgccctgttcacctccaccaaccagctgggcctgaagaccgacgtgaccgactaccacatcgaccaggtgtccaacctggtggagtgcctgtga

SEQ ID NO: 73 - DNA, 1839 bp, synthetic construct
  source 1..1839: mol_type = other DNA
atggaggagaacaaccagaaccagtgcatcccgtacaactgcctgtccaacccggaggaggtgctgctggacggcgagaggatctccaccggcaactcctccatcgacatctccctgtccctggtgcagttcctggtgtccaacttcgtgccgggcggcggcttcctggtgggcctgatcgacttcgtgtggggcatcgtgggcccgtcccagtgggacgccttcctggtgcagatcgagcagctgatcaacgagaggatcgccgagttcgccaggaacgccgccatcgccaacctggagggcctgggcaacaacttcaacatctacgtggaggccttcaaggagtgggaggaggacccgaacaacccggccaccaggaccagggtgatcgacaggttcaggatcctggacggcctgctggagagggacatcccgtccttcaggatctccggcttcgaggtgccgctgctgtccgtgtacgcccaggccgccaacctgcacctggccatcctgagggactccgtgatcttcggcgagaggtggggcctgaccaccatcaacgtgaacgagaactacaacaggctgatcaggcacatcgacgagtacgccgaccactgcgccaacacctacaacaggggcctgaacaacctgccgaagtccacctaccaggactggatcacctacaacaggctgaggagggacctgaccctgaccgtgctggacatcgccgccttcttcccgaactacgacaacaggaggtacccgatccagccggtgggccagctgaccagggaggtgtacaccgacccgctgatcaacttcaacccgcagctgcagtccgtggcccagctgccgaccttcaacgtgatggagtcctccgccatcaggaacccgcacctgttcgacatcctgaacaacctgaccatcttcaccgactggttctccgtgggcaggaacttctactggggcggccacagggtgatctcctccctgatcggcggcggcaacatcacctccccgatctacggcagggaggccaaccaggagccgccgaggtccttcaccttcaacggcccggtgttcaggaccctgtccaacccgaccctgaggctgctgcagcagccgtggccggccccgccgttcaacctgaggggcgtggagggcgtggagttctccaccccgaccaactccttcacctacaggggcaggggcaccgtggactccctgaccgagctgccgccggaggacaactccgtgccgccgagggagggctactcccacaggctgtgccacgccaccttcgtgcagaggtccggcaccccgttcctgaccaccggcgtggtgttctcctggacccacaggtccgccaacaacatcatcccgtcctcccagatcacccagatcccgctgaccaagtccaccaacctgggctccggcacctccgtggtgaagggcccgggcttcaccggcggcgacatcctgaggaggacctccccgggccagatctccaccctgagggtgaacatcaccgccccgctgtcccagaggtacagggtgaggatcaggtacgcctccaccaccaacctgcagttccacacctccatcgacggcaggccgatcaaccagggcaacttctccgccaccatgtcctccggctccaacctgcagtccggctccttcaggaccgtgggcttcaccaccccgttcaacttctccaacggctcctccgtgttcaccctgtccgcccacgtgttcaactccggcaacgaggtgtacatcgacaggatcgagttcgtgccggccgaggtgaccttcgaggccgagtacgacctggagagg

SEQ ID NO: 74 - DNA, 2016 bp, synthetic construct
  source 1..2016: mol_type = other DNA
atggaggagaacaaccagaaccagtgcatcccgtacaactgcctgtccaacccggaggaggtgctgctggacggcgagaggatctccaccggcaactcctccatcgacatctccctgtccctggtgcagttcctggtgtccaacttcgtgccgggcggcggcttcctggtgggcctgatcgacttcgtgtggggcatcgtgggcccgtcccagtgggacgccttcctggtgcagatcgagcagctgatcaacgagaggatcgccgagttcgccaggaacgccgccatcgccaacctggagggcctgggcaacaacttcaacatctacgtggaggccttcaaggagtgggaggaggacccgaacaacccggccaccaggaccagggtgatcgacaggttcaggatcctggacggcctgctggagagggacatcccgtccttcaggatctccggcttcgaggtgccgctgctgtccgtgtacgcccaggccgccaacctgcacctggccatcctgagggactccgtgatcttcggcgagaggtggggcctgaccaccatcaacgtgaacgagaactacaacaggctgatcaggcacatcgacgagtacgccgaccactgcgccaacacctacaacaggggcctgaacaacctgccgaagtccacctaccaggactggatcacctacaacaggctgaggagggacctgaccctgaccgtgctggacatcgccgccttcttcccgaactacgacaacaggaggtacccgatccagccggtgggcaccgtgtcccagctgaccagggagatctacaccaacccggtgctggagaacttcgacggctccttcaggggctccgcccagggcatcgagggctccatcaggtccccgcacctgatggacatcctgaactccatcaccatctacaccgacgcccacaggggcgagtactactggtccggccaccagatcatggcctccccggtgggcttctccggcccggagttcaccttcccgctgtacggcaccatgggcaacgccgccccgcagcagaggatcgtggcccagctgggccagggcgtgtacaggaccctgtcctccaccctgtacaggaggccgttcaacatcggcatcaacaaccagcagctgtccgtgctggacggcaccgagttcgcctacggcacctcctccaacctgccgtccgccgtgtacaggaagtccggcaccgtggactccctggacgagatcccgccgcagaacaacaacgtgccgccgaggcagggcttctcccacaggctgtcccacgtgtccatgttcaggtccggcttctccaactcctccgtgtccatcatcagggccccgatgttctcctggatccacaggtccgccgagttcaccctgaccaacaccatcgacccggagaggatcaaccagatcccgctggtgaagggcttcagggtgtggggcggcacctccgtgatcaccggcccgggcttcaccggcggcgacatcctgaggaggaacaccttcggcgacttcgtgtccctgcaggtgaacatcaactccccgatcacccagaggtacaggctgaggttcaggtacgcctcctccagggacgccagggtgatcgtgctgaccggcgccgcctccaccggcgtgggcggccaggtgtccgtgaacatgccgctgcagaagaccatggagatcggcgagaacctgacctccaggaccttcaggtacaccgacttctccaacccgttctccttcagggccaacccggacatcatcggcatctccgagcagccgctgttcggcgccggctccatctcctccggcgagctgtacatcgacaagatcgagatcatcctggccgacgccaccttcgaggccgagtccgacctggagagggcccagaaggccgtgaacgccctgttcacctcctccaaccagatcggcctgaagaccgacgtgaccgactaccacatcgaccaggtgtccaacctggtggactgcctgtcc

SEQ ID NO: 75 - DNA, 1863 bp, synthetic construct
  source 1..1863: mol_type = other DNA
atggaggagaacaaccagaaccagtgcatcccgtacaactgcctgtccaacccggaggaggtgctgctggacggcgagaggatctccaccggcaactcctccatcgacatctccctgtccctggtgcagttcctggtgtccaacttcgtgccgggcggcggcttcctggtgggcctgatcgacttcgtgtggggcatcgtgggcccgtcccagtgggacgccttcctggtgcagatcgagcagctgatcaacgagaggatcgccgagttcgccaggaacgccgccatcgccaacctggagggcctgggcaacaacttcaacatctacgtggaggccttcaaggagtgggaggaggacccgaacaacccggccaccaggaccagggtgatcgacaggttcaggatcctggacggcctgctggagagggacatcccgtccttcaggatctccggcttcgaggtgccgctgctgtccgtgtacgcccaggccgccaacctgcacctggccatcctgagggactccgtgatcttcggcgagaggtggggcctgaccaccatcaacgtgaacgagaactacaacaggctgatcaggcacatcgacgagtacgccgaccactgcgccaacacctacaacaggggcctgaacaacctgccgaagtccacctaccaggactggatcacctacaacaggctgaggagggacctgaccctgaccgtgctggacatcgccgccttcttcccgaactacgacaacaggaggtacccgatccagccggtgggcaccgtgtcccagctgaccagggagatctacaccaacccggtgctggagaacttcgacggctccttcaggggctccgcccagggcatcgagggctccatcaggtccccgcacctgatggacatcctgaactccatcaccatctacaccgacgcccacaggggcgagtactactggtccggccaccagatcatggcctccccggtgggcttctccggcccggagttcaccttcccgctgtacggcaccatgggcaacgccgccccgcagcagaggatcgtggcccagctgggccagggcgtgtacaggaccctgtcctccaccctgtacaggaggccgttcaacatcggcatcaacaaccagcagctgtccgtgctggacggcaccgagttcgcctacggcacctcctccaacctgccgtccgccgtgtacaggaagtccggcaccgtggactccctggacgagatcccgccgcagaacaacaacgtgccgccgaggcagggcttctcccacaggctgtcccacgtgtccatgttcaggtccggcttctccaactcctccgtgtccatcatcagggccccgatgttctcctggatccacaggtccgccgagttcaacaacatcatcccgtcctcccagatcacccagatcccgctgaccaagtccaccaacctgggctccggcacctccgtggtgaagggcccgggcttcaccggcggcgacatcctgaggaggacctccccgggccagatctccaccctgagggtgaacatcaccgccccgctgtcccagaggtacagggtgaggatcaggtacgcctccaccaccaacctgcagttccacacctccatcgacggcaggccgatcaaccagggcaacttctccgccaccatgtcctccggctccaacctgcagtccggctccttcaggaccgtgggcttcaccaccccgttcaacttctccaacggctcctccgtgttcaccctgtccgcccacgtgttcaactccggcaacgaggtgtacatcgacaggatcgagttcgtgccggccgaggtgaccttcgaggccgagtacgacctggagagg

SEQ ID NO: 76 - DNA, 1986 bp, synthetic construct
  source 1..1986: mol_type = other DNA
atggacaacaacccgaacatcaacgagtgcatcccgtacaactgcctgtccaacccggaggtggaggtgctgggcggcgagaggatcgagaccggctacaccccgatcgacatctccctgtccctgacccagttcctgctgtccgagttcgtgccgggcgccggcttcgtgctgggcctggtggacatcatctggggcatcttcggcccgtcccagtgggacgccttcctggtgcagatcgagcagctgatcaaccagaggatcgaggagttcgccaggaaccaggccatctccaggctggagggcctgtccaacctgtaccagatctacgccgagtccttcagggagtgggaggccgacccgaccaacccggccctgagggaggagatgaggatccagttcaacgacatgaactccgccctgaccaccgccatcccgctgttcgccgtgcagaactaccaggtgccgctgctgtccgtgtacgtgcaggccgccaacctgcacctgtccgtgctgagggacgtgtccgtgttcggccagaggtggggcttcgacgccgccaccatcaactccaggtacaacgacctgaccaggctgatcggcaactacaccgaccacgccgtgaggtggtacaacaccggcctggagagggtgtggggcccggactccagggactggatcaggtacaaccagttcaggagggagctgaccctgaccgtgctggacatcgtgtccctgttcccgaactacgactccaggacctacccgatcaggcagctgaccagggaggtgtacaccgacccgctgatcaacttcaacccgcagctgcagtccgtggcccagctgccgaccttcaacgtgatggagtcctccgccatcaggaacccgcacctgttcgacatcctgaacaacctgaccatcttcaccgactggttctccgtgggcaggaacttctactggggcggccacagggtgatctcctccctgatcggcggcggcaacatcacctccccgatctacggcagggaggccaaccaggagccgccgaggtccttcaccttcaacggcccggtgttcaggaccctgtccaacccgaccctgaggctgctgcagcagccgtggccggccccgccgttcaacctgaggggcgtggagggcgtggagttctccaccccgaccaactccttcacctacaggggcaggggcaccgtggactccctgaccgagctgccgccggaggacaactccgtgccgccgagggagggctactcccacaggctgtgccacgccaccttcgtgcagaggtccggcaccccgttcctgaccaccggcgtggtgttctcctggacccacaggtccgccaccctgaccaacaccatcgacccggagaggatcaaccagatcccgctggtgaagggcttcagggtgtggggcggcacctccgtgatcaccggcccgggcttcaccggcggcgacatcctgaggaggaacaccttcggcgacttcgtgtccctgcaggtgaacatcaactccccgatcacccagaggtacaggctgaggttcaggtacgcctcctccagggacgccagggtgatcgtgctgaccggcgccgcctccaccggcgtgggcggccaggtgtccgtgaacatgccgctgcagaagaccatggagatcggcgagaacctgacctccaggaccttcaggtacaccgacttctccaacccgttctccttcagggccaacccggacatcatcggcatctccgagcagccgctgttcggcgccggctccatctcctccggcgagctgtacatcgacaagatcgagatcatcctggccgacgccaccttcgaggccgagtccgacctggagagggcccagaaggccgtgaacgccctgttcacctcctccaaccagatcggcctgaagaccgacgtgaccgactaccacatcgaccaggtgtccaacctggtggactgcctgtcc

SEQ ID NO: 77 - DNA, 1833 bp, synthetic construct
  source 1..1833: mol_type = other DNA
atggacaacaacccgaacatcaacgagtgcatcccgtacaactgcctgtccaacccggaggtggaggtgctgggcggcgagaggatcgagaccggctacaccccgatcgacatctccctgtccctgacccagttcctgctgtccgagttcgtgccgggcgccggcttcgtgctgggcctggtggacatcatctggggcatcttcggcccgtcccagtgggacgccttcctggtgcagatcgagcagctgatcaaccagaggatcgaggagttcgccaggaaccaggccatctccaggctggagggcctgtccaacctgtaccagatctacgccgagtccttcagggagtgggaggccgacccgaccaacccggccctgagggaggagatgaggatccagttcaacgacatgaactccgccctgaccaccgccatcccgctgttcgccgtgcagaactaccaggtgccgctgctgtccgtgtacgtgcaggccgccaacctgcacctgtccgtgctgagggacgtgtccgtgttcggccagaggtggggcttcgacgccgccaccatcaactccaggtacaacgacctgaccaggctgatcggcaactacaccgaccacgccgtgaggtggtacaacaccggcctggagagggtgtggggcccggactccagggactggatcaggtacaaccagttcaggagggagctgaccctgaccgtgctggacatcgtgtccctgttcccgaactacgactccaggacctacccgatcaggcagctgaccagggaggtgtacaccgacccgctgatcaacttcaacccgcagctgcagtccgtggcccagctgccgaccttcaacgtgatggagtcctccgccatcaggaacccgcacctgttcgacatcctgaacaacctgaccatcttcaccgactggttctccgtgggcaggaacttctactggggcggccacagggtgatctcctccctgatcggcggcggcaacatcacctccccgatctacggcagggaggccaaccaggagccgccgaggtccttcaccttcaacggcccggtgttcaggaccctgtccaacccgaccctgaggctgctgcagcagccgtggccggccccgccgttcaacctgaggggcgtggagggcgtggagttctccaccccgaccaactccttcacctacaggggcaggggcaccgtggactccctgaccgagctgccgccggaggacaactccgtgccgccgagggagggctactcccacaggctgtgccacgccaccttcgtgcagaggtccggcaccccgttcctgaccaccggcgtggtgttctcctggacccacaggtccgccaacaacatcatcccgtcctcccagatcacccagatcccgctgaccaagtccaccaacctgggctccggcacctccgtggtgaagggcccgggcttcaccggcggcgacatcctgaggaggacctccccgggccagatctccaccctgagggtgaacatcaccgccccgctgtcccagaggtacagggtgaggatcaggtacgcctccaccaccaacctgcagttccacacctccatcgacggcaggccgatcaaccagggcaacttctccgccaccatgtcctccggctccaacctgcagtccggctccttcaggaccgtgggcttcaccaccccgttcaacttctccaacggctcctccgtgttcaccctgtccgcccacgtgttcaactccggcaacgaggtgtacatcgacaggatcgagttcgtgccggccgaggtgaccttcgaggccgagtacgacctggagagg

SEQ ID NO: 78 - DNA, 2010 bp, synthetic construct
  source 1..2010: mol_type = other DNA
atggacaacaacccgaacatcaacgagtgcatcccgtacaactgcctgtccaacccggaggtggaggtgctgggcggcgagaggatcgagaccggctacaccccgatcgacatctccctgtccctgacccagttcctgctgtccgagttcgtgccgggcgccggcttcgtgctgggcctggtggacatcatctggggcatcttcggcccgtcccagtgggacgccttcctggtgcagatcgagcagctgatcaaccagaggatcgaggagttcgccaggaaccaggccatctccaggctggagggcctgtccaacctgtaccagatctacgccgagtccttcagggagtgggaggccgacccgaccaacccggccctgagggaggagatgaggatccagttcaacgacatgaactccgccctgaccaccgccatcccgctgttcgccgtgcagaactaccaggtgccgctgctgtccgtgtacgtgcaggccgccaacctgcacctgtccgtgctgagggacgtgtccgtgttcggccagaggtggggcttcgacgccgccaccatcaactccaggtacaacgacctgaccaggctgatcggcaactacaccgaccacgccgtgaggtggtacaacaccggcctggagagggtgtggggcccggactccagggactggatcaggtacaaccagttcaggagggagctgaccctgaccgtgctggacatcgtgtccctgttcccgaactacgactccaggacctacccgatcaggaccgtgtcccagctgaccagggagatctacaccaacccggtgctggagaacttcgacggctccttcaggggctccgcccagggcatcgagggctccatcaggtccccgcacctgatggacatcctgaactccatcaccatctacaccgacgcccacaggggcgagtactactggtccggccaccagatcatggcctccccggtgggcttctccggcccggagttcaccttcccgctgtacggcaccatgggcaacgccgccccgcagcagaggatcgtggcccagctgggccagggcgtgtacaggaccctgtcctccaccctgtacaggaggccgttcaacatcggcatcaacaaccagcagctgtccgtgctggacggcaccgagttcgcctacggcacctcctccaacctgccgtccgccgtgtacaggaagtccggcaccgtggactccctggacgagatcccgccgcagaacaacaacgtgccgccgaggcagggcttctcccacaggctgtcccacgtgtccatgttcaggtccggcttctccaactcctccgtgtccatcatcagggccccgatgttctcctggatccacaggtccgccgagttcaccctgaccaacaccatcgacccggagaggatcaaccagatcccgctggtgaagggcttcagggtgtggggcggcacctccgtgatcaccggcccgggcttcaccggcggcgacatcctgaggaggaacaccttcggcgacttcgtgtccctgcaggtgaacatcaactccccgatcacccagaggtacaggctgaggttcaggtacgcctcctccagggacgccagggtgatcgtgctgaccggcgccgcctccaccggcgtgggcggccaggtgtccgtgaacatgccgctgcagaagaccatggagatcggcgagaacctgacctccaggaccttcaggtacaccgacttctccaacccgttctccttcagggccaacccggacatcatcggcatctccgagcagccgctgttcggcgccggctccatctcctccggcgagctgtacatcgacaagatcgagatcatcctggccgacgccaccttcgaggccgagtccgacctggagagggcccagaaggccgtgaacgccctgttcacctcctccaaccagatcggcctgaagaccgacgtgaccgactaccacatcgaccaggtgtccaacctggtggactgcctgtcc

SEQ ID NO: 79 - DNA, 1920 bp, synthetic construct
  source 1..1920: mol_type = other DNA
atgaagaacaacatccagaaccagtgcgtgccgtacaactgcctgtccaacccggaggtggagatcctgtccgaggagaggtccaccggcaggctgccgctggacatctccctgtccctgaccaggttcctgctgtccgagttcgtgccgggcgtgggcgtggccttcggcctgttcgacctgatctggggcttcatcaccccgtccgagtggtccctgttcctgctgcagatcgagcagctgatcgagcagaggatcgagaccctggagaggaacagggccatcaccaccctgaggggcctggccgactcctacgaggtgtacctggaggccctgagggagtgggaggagaacccgaacaacgcccagctgagggaggacgtgaggatcaggttcgccaacaccgacgacgccctgatcaccgccatcaacaacttcaccctgacctccttcgagatcccgctgctgtccgtgtacgtgcaggccgccaacctgcacctgtccctgctgagggacgccgtgtccttcggccagggctggggcctggacatcgccaccgtgaacaaccactacaacaggctgatcaacctgatccacaggtacaccgagcactgcctggacacctacaaccagggcctggagaacctgaggggcaccaacaccaggcagtggtccaggttcaaccagttcaggagggagctgaccctgaccgtgctggacatcgtggccctgttcccgaactacgacgccagggcctacccgatccagaccgccacccagctgtccagggaggtgtacctggacctgccgttcatcaacgagaacctgtccccggccgcctcctacccgaccttctccgccgccgagtccgccatcatcaggtccccgcacctggtggacttcctgaacaacttcaccatctacaccgactccctggcctcctacgcctactggggcggccacagggtgaggtccttcaggaccggcaccaccaccaacctgatccagtccccgctgtacggcagggagggcaacaccgagaggccggtgaccatctgggcctcctcctccgtgccgatcttcaggaccctgtcctacgtgaccggcctgaacaacaggaacccggtggccggcatcgagggcgtggagttccaggacaccaccggcaggtccatctacaggaagtccggcccgatcgactccttctccgagctgccgccgcaggacgccaacatctccccgaccatcggctactcccacaggctgtgccacgccaccttcctggagaggatctccggcccgaggatcgccggcgtggtgttctcctggacccacaggtccgcctccccgaccaacgaggtgtccccgtccaggatcacccagatcccgtgggtgaaggccaacaccctggcctccggcgcctccgtgatcaagggcccgggcttcaccggcggcgacatcctgaccaggaactccatgggcgacctgggctccctgagggtgaccttcaccggcaggctgccgcagtcctactacatcaggttcaggtacgcctccgtggccaacaggtccggctccttcaggtactcccagccgccgtcctacggcatctccttcccgaagaccatggacgccggcgagccgctgacctccaggtccttcgccctgaccaccctgttcaccccgatcaccttctccagggcccaggaggagttcgacctgtacatccagaccggcgtgtacatcgacaggatcgagttcatcccggtgaccgccaccctggaggccgagtacgacctggagagggcccaggaggccgtgaacgccctgttcacctcctccaaccagctgggcctgaagaccgacgtgaccgactaccacatcgaccaggtgtccaacctggtggactgcctgtcctag

SEQ ID NO: 80 - DNA, 1932 bp, synthetic construct
  source 1..1932: mol_type = other DNA
atggagatcaacaaccagaaccagtgcgtgccgtacaactgcctgtccaacccgaaggagatcatcctgggcgaggagaggctggagaccggcaacaccgtggccgacatctccctgggcctgatcaacttcctgtactccaacttcgtgccgggcggcggcttcatcgtgggcctgctggagctgatctggggcttcatcggcccgtcccagtgggacatcttcctggcccagatcgagcagctgatctcccagaggatcgaggagttcgccaggaaccaggccatctccaggctggagggcctgtccaacctgtacaaggtgtacgccagggccttctccgactgggaggaggacccgaccaacccggccctgagggaggagatgaggatccagttcaacgacatgaactccgccctgatcaccgccatcccgctgttcagggtgcagaactacgaggtggccctgctgtccgtgtacgtgcaggccgccaacctgcacctgtccatcctgagggacatctccgtgttcggcgagaggtggggctacgacaccgccaccatcaacaacaggtactccgacctgacctccctgatccacgtgtacaccaaccactgcgtggacacctacaaccagggcctgaagctgctggagggcaggttcctgtccgactggatcgtgtacaacaggttcaggaggcagctgaccatctccgtgctggacatcgtggccttcttcccgaactacgacatcagggcctacccgatccagaccgccacccagctgtccagggaggtgtacctggacctgccgttcatcaacgagaacctgtccccggccgcctcctacccgaccttctccgccgccgagtccgccatcatcaggtccccgcacctggtggacttcctgaacaacttcaccatctacaccgactccctggcctcctacgcctactggggcggccacagggtgaggtccttcaggaccggcaccaccaccaacctgatccagtccccgctgtacggcagggagggcaacaccgagaggccggtgaccatctgggcctcctcctccgtgccgatcttcaggaccctgtcctacgtgaccggcctgaacaacaggaacccggtggccggcatcgagggcgtggagttccaggacaccaccggcaggtccatctacaggaagtccggcccgatcgactccttctccgagctgccgccgcaggacgccaacatctccccgaccatcggctactcccacaggctgtgccacgccaccttcctggagaggatctccggcccgaggatcgccggcgtggtgttctcctggacccacaggtccgcctccccgaccaacgaggtgtccccgtccaggatcacccagatcccggccgtgaaggcccacaacctgcactccggctccaccgtggtgaggggcccgggcttcaccggcggcgacctgctgaggaggaccaacaccggcaccttcgccgacatcagggtgaacatcaccggcccgctgtcccagaggtacagggtgaggatcaggtacgcctccaccaccgacctgcagttcttcaccaggatcaacggcacctccgtgaaccagggcaacttccagaggaccatgaacaggggcggcaacctggagtccggcaacttcaggaccgccggcttctccaccccgttctccttctccaacgcccagtccaccttcaccctgggcacccaggccttctccaaccaggaggtgtacatcgacaggatcgagttcgtgccggccgaggtgaccttcgaggccgagtccgacctggagagggcccagaaggccgtgaacgccctgttcacctccacctcccagctgggcctgaagaccaacgtgaccggctaccacatcgaccaggtgtccaacctggtggcctgcctgtcc

SEQ ID NO: 81 - DNA, 1935 bp, synthetic construct
  source 1..1935: mol_type = other DNA
atggagatcaacaaccagaaccagtgcgtgccgtacaactgcctgtccaacccgaaggagatcatcctgggcgaggagaggctggagaccggcaacaccgtggccgacatctccctgggcctgatcaacttcctgtactccaacttcgtgccgggcggcggcttcatcgtgggcctgctggagctgatctggggcttcatcggcccgtcccagtgggacatcttcctggcccagatcgagcagctgatctcccagaggatcgaggagttcgccaggaaccaggccatctccaggctggagggcctgtccaacctgtacaaggtgtacgccagggccttctccgactgggaggaggacccgaccaacccggccctgagggaggagatgaggatccagttcaacgacatgaactccgccctgatcaccgccatcccgctgttcagggtgcagaactacgaggtggccctgctgtccgtgtacgtgcaggccgccaacctgcacctgtccatcctgagggacatctccgtgttcggcgagaggtggggctacgacaccgccaccatcaacaacaggtactccgacctgacctccctgatccacgtgtacaccaaccactgcgtggacacctacaaccagggcctgaagctgctggagggcaggttcctgtccgactggatcgtgtacaacaggttcaggaggcagctgaccatctccgtgctggacatcgtggccttcttcccgaactacgacatcagggcctacccgatccagacctcctcccagctgaccagggagatctacacctcctccgtgatcgaggactccccggtgtccgccaacatcccgaacggcttcaacagggccgagttcggcgtgaggccgccgcacctgatggacttcatgaactccctgttcgtgaccgccgagaccgtgaggtcccagaccgtgtggggcggccacctggtgtcctccaggaacaccgccggcaacccgatcaacttcccgatctacggcatcttcaacccgggcggcgccatctggatcgccgacgaggacccgaggccgttctacaggaccctgtccgacccggtgttcgtgaggggcggcttcggcaacccgcactacgtgctgggcctgaggggcgtggccttccagcagaccggcaccaaccacaccaggaccttcaggaactccggcaccatcgactccctggacgagatcccgccgcaggacaactccggcgccccgtggaacgactactcccacgtgctgaaccacgtgaccttcgtgaggtggccgggcgagatcgccggctccgactcctggagggccccgatgttctcctggacccacaggtccgccgacaggaccaacatcatcaacccgaacatcatcacccagatcccgtgggtgaaggccaacaccctggcctccggcgcctccgtgatcaagggcccgggcttcaccggcggcgacatcctgaccaggaactccatgggcgacctgggctccctgagggtgaccttcaccggcaggctgccgcagtcctactacatcaggttcaggtacgcctccgtggccaacaggtccggctccttcaggtactcccagccgccgtcctacggcatctccttcccgaagaccatggacgccggcgagccgctgacctccaggtccttcgccctgaccaccctgttcaccccgatcaccttctccagggcccaggaggagttcgacctgtacatccagaccggcgtgtacatcgacaggatcgagttcatcccggtgaccgccaccctggaggccgagtacgacctggagagggcccaggaggccgtgaacgccctgttcacctcctccaaccagctgggcctgaagaccgacgtgaccgactaccacatcgaccaggtgtccaacctggtggactgcctgtcc

SEQ ID NO: 82 - DNA, 1947 bp, synthetic construct
  source 1..1947: mol_type = other DNA
atggagatcaacaaccagaaccagtgcgtgccgtacaactgcctgtccaacccgaaggagatcatcctgggcgaggagaggctggagaccggcaacaccgtggccgacatctccctgggcctgatcaacttcctgtactccaacttcgtgccgggcggcggcttcatcgtgggcctgctggagctgatctggggcttcatcggcccgtcccagtgggacatcttcctggcccagatcgagcagctgatctcccagaggatcgaggagttcgccaggaaccaggccatctccaggctggagggcctgtccaacctgtacaaggtgtacgccagggccttctccgactgggaggaggacccgaccaacccggccctgagggaggagatgaggatccagttcaacgacatgaactccgccctgatcaccgccatcccgctgttcagggtgcagaactacgaggtggccctgctgtccgtgtacgtgcaggccgccaacctgcacctgtccatcctgagggacatctccgtgttcggcgagaggtggggctacgacaccgccaccatcaacaacaggtactccgacctgacctccctgatccacgtgtacaccaaccactgcgtggacacctacaaccagggcctgaagctgctggagggcaggttcctgtccgactggatcgtgtacaacaggttcaggaggcagctgaccatctccgtgctggacatcgtggccttcttcccgaactacgacatcagggcctacccgatccagacctcctcccagctgaccagggagatctacacctcctccgtgatcgaggactccccggtgtccgccaacatcccgaacggcttcaacagggccgagttcggcgtgaggccgccgcacctgatggacttcatgaactccctgttcgtgaccgccgagaccgtgaggtcccagaccgtgtggggcggccacctggtgtcctccaggaacaccgccggcaacccgatcaacttcccgatctacggcatcttcaacccgggcggcgccatctggatcgccgacgaggacccgaggccgttctacaggaccctgtccgacccggtgttcgtgaggggcggcttcggcaacccgcactacgtgctgggcctgaggggcgtggccttccagcagaccggcaccaaccacaccaggaccttcaggaactccggcaccatcgactccctggacgagatcccgccgcaggacaactccggcgccccgtggaacgactactcccacgtgctgaaccacgtgaccttcgtgaggtggccgggcgagatcgccggctccgactcctggagggccccgatgttctcctggacccacaggtccgccgacaggaccaacatcatcaacccgaacatcatcacccagatcccggccgtgaaggcccacaacctgcactccggctccaccgtggtgaggggcccgggcttcaccggcggcgacctgctgaggaggaccaacaccggcaccttcgccgacatcagggtgaacatcaccggcccgctgtcccagaggtacagggtgaggatcaggtacgcctccaccaccgacctgcagttcttcaccaggatcaacggcacctccgtgaaccagggcaacttccagaggaccatgaacaggggcggcaacctggagtccggcaacttcaggaccgccggcttctccaccccgttctccttctccaacgcccagtccaccttcaccctgggcacccaggccttctccaaccaggaggtgtacatcgacaggatcgagttcgtgccggccgaggtgaccttcgaggccgagtccgacctggagagggcccagaaggccgtgaacgccctgttcacctccacctcccagctgggcctgaagaccaacgtgaccggctaccacatcgaccaggtgtccaacctggtggcctgcctgtcc

SEQ ID NO: 83 - DNA, 1929 bp, synthetic construct
  source 1..1929: mol_type = other DNA
atgaagaacaacatccagaaccagtgcgtgccgtacaactgcctgtccaacccggaggtggagatcctgtccgaggagaggtccaccggcaggctgccgctggacatctccctgtccctgaccaggttcctgctgtccgagttcgtgccgggcgtgggcgtggccttcggcctgttcgacctgatctggggcttcatcaccccgtccgagtggtccctgttcctgctgcagatcgagcagctgatcgagcagaggatcgagaccctggagaggaacagggccatcaccaccctgaggggcctggccgactcctacgaggtgtacctggaggccctgagggagtgggaggagaacccgaacaacgcccagctgagggaggacgtgaggatcaggttcgccaacaccgacgacgccctgatcaccgccatcaacaacttcaccctgacctccttcgagatcccgctgctgtccgtgtacgtgcaggccgccaacctgcacctgtccctgctgagggacgccgtgtccttcggccagggctggggcctggacatcgccaccgtgaacaaccactacaacaggctgatcaacctgatccacaggtacaccgagcactgcctggacacctacaaccagggcctggagaacctgaggggcaccaacaccaggcagtggtccaggttcaaccagttcaggagggagctgaccctgaccgtgctggacatcgtggccctgttcccgaactacgacgccagggcctacccgatccagaccgccacccagctgtccagggaggtgtacctggacctgccgttcatcaacgagaacctgtccccggccgcctcctacccgaccttctccgccgccgagtccgccatcatcaggtccccgcacctggtggacttcctgaacaacttcaccatctacaccgactccctggcctcctacgcctactggggcggccacagggtgaggtccttcaggaccggcaccaccaccaacctgatccagtccccgctgtacggcagggagggcaacaccgagaggccggtgaccatctgggcctcctcctccgtgccgatcttcaggaccctgtcctacgtgaccggcctgaacaacaggaacccggtggccggcatcgagggcgtggagttccaggacaccaccggcaggtccatctacaggaagtccggcccgatcgactccttctccgagctgccgccgcaggacgccaacatctccccgaccatcggctactcccacaggctgtgccacgccaccttcctggagaggatctccggcccgaggatcgccggcgtggtgttctcctggacccacaggtccgcctccccgaccaacgaggtgtccccgtccaggatcacccagatcccggccgtgaaggcccacaacctgcactccggctccaccgtggtgaggggcccgggcttcaccggcggcgacctgctgaggaggaccaacaccggcaccttcgccgacatcagggtgaacatcaccggcccgctgtcccagaggtacagggtgaggatcaggtacgcctccaccaccgacctgcagttcttcaccaggatcaacggcacctccgtgaaccagggcaacttccagaggaccatgaacaggggcggcaacctggagtccggcaacttcaggaccgccggcttctccaccccgttctccttctccaacgcccagtccaccttcaccctgggcacccaggccttctccaaccaggaggtgtacatcgacaggatcgagttcgtgccggccgaggtgaccttcgaggccgagtccgacctggagagggcccagaaggccgtgaacgccctgttcacctccacctcccagctgggcctgaagaccaacgtgaccggctaccacatcgaccaggtgtccaacctggtggcctgcctgtcc

SEQ ID NO: 84 - DNA, 1932 bp, synthetic construct
  source 1..1932: mol_type = other DNA
atgaagaacaacatccagaaccagtgcgtgccgtacaactgcctgtccaacccggaggtggagatcctgtccgaggagaggtccaccggcaggctgccgctggacatctccctgtccctgaccaggttcctgctgtccgagttcgtgccgggcgtgggcgtggccttcggcctgttcgacctgatctggggcttcatcaccccgtccgagtggtccctgttcctgctgcagatcgagcagctgatcgagcagaggatcgagaccctggagaggaacagggccatcaccaccctgaggggcctggccgactcctacgaggtgtacctggaggccctgagggagtgggaggagaacccgaacaacgcccagctgagggaggacgtgaggatcaggttcgccaacaccgacgacgccctgatcaccgccatcaacaacttcaccctgacctccttcgagatcccgctgctgtccgtgtacgtgcaggccgccaacctgcacctgtccctgctgagggacgccgtgtccttcggccagggctggggcctggacatcgccaccgtgaacaaccactacaacaggctgatcaacctgatccacaggtacaccgagcactgcctggacacctacaaccagggcctggagaacctgaggggcaccaacaccaggcagtggtccaggttcaaccagttcaggagggagctgaccctgaccgtgctggacatcgtggccctgttcccgaactacgacgccagggcctacccgatccagacctcctcccagctgaccagggagatctacacctcctccgtgatcgaggactccccggtgtccgccaacatcccgaacggcttcaacagggccgagttcggcgtgaggccgccgcacctgatggacttcatgaactccctgttcgtgaccgccgagaccgtgaggtcccagaccgtgtggggcggccacctggtgtcctccaggaacaccgccggcaacccgatcaacttcccgatctacggcatcttcaacccgggcggcgccatctggatcgccgacgaggacccgaggccgttctacaggaccctgtccgacccggtgttcgtgaggggcggcttcggcaacccgcactacgtgctgggcctgaggggcgtggccttccagcagaccggcaccaaccacaccaggaccttcaggaactccggcaccatcgactccctggacgagatcccgccgcaggacaactccggcgccccgtggaacgactactcccacgtgctgaaccacgtgaccttcgtgaggtggccgggcgagatcgccggctccgactcctggagggccccgatgttctcctggacccacaggtccgccgacaggaccaacatcatcaacccgaacatcatcacccagatcccgtgggtgaaggccaacaccctggcctccggcgcctccgtgatcaagggcccgggcttcaccggcggcgacatcctgaccaggaactccatgggcgacctgggctccctgagggtgaccttcaccggcaggctgccgcagtcctactacatcaggttcaggtacgcctccgtggccaacaggtccggctccttcaggtactcccagccgccgtcctacggcatctccttcccgaagaccatggacgccggcgagccgctgacctccaggtccttcgccctgaccaccctgttcaccccgatcaccttctccagggcccaggaggagttcgacctgtacatccagaccggcgtgtacatcgacaggatcgagttcatcccggtgaccgccaccctggaggccgagtacgacctggagagggcccaggaggccgtgaacgccctgttcacctcctccaaccagctgggcctgaagaccgacgtgaccgactaccacatcgaccaggtgtccaacctggtggactgcctgtcc

SEQ ID NO: 85 - DNA, 1983 bp, synthetic construct
  source 1..1983: mol_type = other DNA
atggagatcgtgaacaaccagaaccagtgcgtgccgtacaactgcctgaacaacccggagaacgagatcctggacatcgagaggtccaactccaccgtggccaccaacatcgccctggagatctccaggctgctggcctccgccaccccgatcggcggcatcctgctgggcctgttcgacgccatctggggctccatcggcccgtcccagtgggacctgttcctggagcagatcgagctgctgatcgaccagaagatcgaggagttcgccaggaaccaggccatctccaggctggagggcatctcctccctgtacggcatctacaccgaggccttcagggagtgggaggccgacccgaccaacccggccctgaaggaggagatgaggacccagttcaacgacatgaactccatcctggtgaccgccatcccgctgttctccgtgcagaactaccaggtgccgttcctgtccgtgtacgtgcaggccgccaacctgcacctgtccgtgctgagggacgtgtccgtgttcggccaggcctggggcttcgacatcgccaccatcaactccaggtacaacgacctgaccaggctgatcccgatctacaccgactacgccgtgaggtggtacaacaccggcctggacaggctgccgaggaccggcggcctgaggaactgggccaggttcaaccagttcaggagggagctgaccatctccgtgctggacatcatctccttcttcaggaactacgactccaggctgtacccgatcccgacctcctcccagctgaccagggaggtgtacaccgacccggtgatcaacatcaccgactacagggtgggcccgtccttcgagaacatcgagaactccgccatcaggtccccgcacctgatggacttcctgaacaacctgaccatcgacaccgacctgatcaggggcgtgcactactgggccggccacagggtgacctcccacttcaccggctcctcccaggtgatcaccaccccgcagtacggcatcaccgccaacgccgagccgaggaggaccatcgccccgtccaccttcccgggcctgaacctgttctacaggaccctgtccaacccgttcttcaggaggtccgagaacatcaccccgaccctgggcatcaacgtggtgcagggcgtgggcttcatccagccgaacaacgccgaggtgctgtacaggtccaggggcaccgtggactccctgaacgagctgccgatcgacggcgagaactccctggtgggctactcccacaggctgtcccacgtgaccctgaccaggtccctgtacaacaccaacatcacctccctgccgaccttcgtgtggacccaccactccgccaccctgaccaacaccatcgacccggagaggatcaaccagatcccgctggtgaagggcttcagggtgtggggcggcacctccgtgatcaccggcccgggcttcaccggcggcgacatcctgaggaggaacaccttcggcgacttcgtgtccctgcaggtgaacatcaactccccgatcacccagaggtacaggctgaggttcaggtacgcctcctccagggacgccagggtgatcgtgctgaccggcgccgcctccaccggcgtgggcggccaggtgtccgtgaacatgccgctgcagaagaccatggagatcggcgagaacctgacctccaggaccttcaggtacaccgacttctccaacccgttctccttcagggccaacccggacatcatcggcatctccgagcagccgctgttcggcgccggctccatctcctccggcgagctgtacatcgacaagatcgagatcatcctggccgacgccaccttcgaggccgagtccgacctggagagggcccagaaggccgtgaacgccctgttcacctcctccaaccagatcggcctgaagaccgacgtgaccgactaccacatcgaccaggtgtccaacctggtggactgcctgtcc

SEQ ID NO: 86 - DNA, 1944 bp, synthetic construct
  source 1..1944: mol_type = other DNA
atggagatcgtgaacaaccagaaccagtgcgtgccgtacaactgcctgaacaacccggagaacgagatcctggacatcgagaggtccaactccaccgtggccaccaacatcgccctggagatctccaggctgctggcctccgccaccccgatcggcggcatcctgctgggcctgttcgacgccatctggggctccatcggcccgtcccagtgggacctgttcctggagcagatcgagctgctgatcgaccagaagatcgaggagttcgccaggaaccaggccatctccaggctggagggcatctcctccctgtacggcatctacaccgaggccttcagggagtgggaggccgacccgaccaacccggccctgaaggaggagatgaggacccagttcaacgacatgaactccatcctggtgaccgccatcccgctgttctccgtgcagaactaccaggtgccgttcctgtccgtgtacgtgcaggccgccaacctgcacctgtccgtgctgagggacgtgtccgtgttcggccaggcctggggcttcgacatcgccaccatcaactccaggtacaacgacctgaccaggctgatcccgatctacaccgactacgccgtgaggtggtacaacaccggcctggacaggctgccgaggaccggcggcctgaggaactgggccaggttcaaccagttcaggagggagctgaccatctccgtgctggacatcatctccttcttcaggaactacgactccaggctgtacccgatcccgcagctgaccagggaggtgtacaccgacccgctgatcaacttcaacccgcagctgcagtccgtggcccagctgccgaccttcaacgtgatggagtcctccgccatcaggaacccgcacctgttcgacatcctgaacaacctgaccatcttcaccgactggttctccgtgggcaggaacttctactggggcggccacagggtgatctcctccctgatcggcggcggcaacatcacctccccgatctacggcagggaggccaaccaggagccgccgaggtccttcaccttcaacggcccggtgttcaggaccctgtccaacccgaccctgaggctgctgcagcagccgtggccggccccgccgttcaacctgaggggcgtggagggcgtggagttctccaccccgaccaactccttcacctacaggggcaggggcaccgtggactccctgaccgagctgccgccggaggacaactccgtgccgccgagggagggctactcccacaggctgtgccacgccaccttcgtgcagaggtccggcaccccgttcctgaccaccggcgtggtgttctcctggacccacaggtccgccaccaacaccaacaccatcaacccggacatcatcacccagatcccgctggtgaagggcttcaggctgggcggcggcacctccgtgatcaagggcccgggcttcaccggcggcgacatcctgaggaggaacaccatcggcgagttcgtgtccctgcaggtgaacatcaactccccgatcacccagaggtacaggctgaggttcaggtacgcctcctccagggacgccaggatcaccgtggccatcggcggccagatcagggtggacatgaccctggagaagaccatggagatcggcgagtccctgacctccaggaccttctcctacaccaacttctccaacccgttctccttcagggccaacccggacatcatcaggatcgccgaggagctgccgatcaggggcggcgagctgtacatcgacaagatcgagctgatcctggccgacgccaccttcgaggaggagtacgacctggagagggcccagaaggccgtgaacgccctgttcacctccaccaaccagctgggcctgaagaccgacgtgaccgactaccacatcgaccaggtgtccaacctggtggagtgcctg

SEQ ID NO: 87 - DNA, 1983 bp, synthetic construct
  source 1..1983: mol_type = other DNA
atggagatcgtgaacaaccagaaccagtgcgtgccgtacaactgcctgaacaacccggagaacgagatcctggacatcgagaggtccaactccaccgtggccaccaacatcgccctggagatctccaggctgctggcctccgccaccccgatcggcggcatcctgctgggcctgttcgacgccatctggggctccatcggcccgtcccagtgggacctgttcctggagcagatcgagctgctgatcgaccagaagatcgaggagttcgccaggaaccaggccatctccaggctggagggcatctcctccctgtacggcatctacaccgaggccttcagggagtgggaggccgacccgaccaacccggccctgaaggaggagatgaggacccagttcaacgacatgaactccatcctggtgaccgccatcccgctgttctccgtgcagaactaccaggtgccgttcctgtccgtgtacgtgcaggccgccaacctgcacctgtccgtgctgagggacgtgtccgtgttcggccaggcctggggcttcgacatcgccaccatcaactccaggtacaacgacctgaccaggctgatcccgatctacaccgactacgccgtgaggtggtacaacaccggcctggacaggctgccgaggaccggcggcctgaggaactgggccaggttcaaccagttcaggagggagctgaccatctccgtgctggacatcatctccttcttcaggaactacgactccaggctgtacccgatcccgcagctgaccagggaggtgtacaccgacccgctgatcaacttcaacccgcagctgcagtccgtggcccagctgccgaccttcaacgtgatggagtcctccgccatcaggaacccgcacctgttcgacatcctgaacaacctgaccatcttcaccgactggttctccgtgggcaggaacttctactggggcggccacagggtgatctcctccctgatcggcggcggcaacatcacctccccgatctacggcagggaggccaaccaggagccgccgaggtccttcaccttcaacggcccggtgttcaggaccctgtccaacccgaccctgaggctgctgcagcagccgtggccggccccgccgttcaacctgaggggcgtggagggcgtggagttctccaccccgaccaactccttcacctacaggggcaggggcaccgtggactccctgaccgagctgccgccggaggacaactccgtgccgccgagggagggctactcccacaggctgtgccacgccaccttcgtgcagaggtccggcaccccgttcctgaccaccggcgtggtgttctcctggacccacaggtccgccaccctgaccaacaccatcgacccggagaggatcaaccagatcccgctggtgaagggcttcagggtgtggggcggcacctccgtgatcaccggcccgggcttcaccggcggcgacatcctgaggaggaacaccttcggcgacttcgtgtccctgcaggtgaacatcaactccccgatcacccagaggtacaggctgaggttcaggtacgcctcctccagggacgccagggtgatcgtgctgaccggcgccgcctccaccggcgtgggcggccaggtgtccgtgaacatgccgctgcagaagaccatggagatcggcgagaacctgacctccaggaccttcaggtacaccgacttctccaacccgttctccttcagggccaacccggacatcatcggcatctccgagcagccgctgttcggcgccggctccatctcctccggcgagctgtacatcgacaagatcgagatcatcctggccgacgccaccttcgaggccgagtccgacctggagagggcccagaaggccgtgaacgccctgttcacctcctccaaccagatcggcctgaagaccgacgtgaccgactaccacatcgaccaggtgtccaacctggtggactgcctgtcc

SEQ ID NO: 88 - DNA, 1944 bp, synthetic construct
  source 1..1944: mol_type = other DNA
atggaggagaacaaccagaaccagtgcatcccgtacaactgcctgtccaacccggaggaggtgctgctggacggcgagaggatctccaccggcaactcctccatcgacatctccctgtccctggtgcagttcctggtgtccaacttcgtgccgggcggcggcttcctggtgggcctgatcgacttcgtgtggggcatcgtgggcccgtcccagtgggacgccttcctggtgcagatcgagcagctgatcaacgagaggatcgccgagttcgccaggaacgccgccatcgccaacctggagggcctgggcaacaacttcaacatctacgtggaggccttcaaggagtgggaggaggacccgaacaacccggccaccaggaccagggtgatcgacaggttcaggatcctggacggcctgctggagagggacatcccgtccttcaggatctccggcttcgaggtgccgctgctgtccgtgtacgcccaggccgccaacctgcacctggccatcctgagggactccgtgatcttcggcgagaggtggggcctgaccaccatcaacgtgaacgagaactacaacaggctgatcaggcacatcgacgagtacgccgaccactgcgccaacacctacaacaggggcctgaacaacctgccgaagtccacctaccaggactggatcacctacaacaggctgaggagggacctgaccctgaccgtgctggacatcgccgccttcttcccgaactacgacaacaggaggtacccgatcccgacctcctcccagctgaccagggaggtgtacaccgacccggtgatcaacatcaccgactacagggtgggcccgtccttcgagaacatcgagaactccgccatcaggtccccgcacctgatggacttcctgaacaacctgaccatcgacaccgacctgatcaggggcgtgcactactgggccggccacagggtgacctcccacttcaccggctcctcccaggtgatcaccaccccgcagtacggcatcaccgccaacgccgagccgaggaggaccatcgccccgtccaccttcccgggcctgaacctgttctacaggaccctgtccaacccgttcttcaggaggtccgagaacatcaccccgaccctgggcatcaacgtggtgcagggcgtgggcttcatccagccgaacaacgccgaggtgctgtacaggtccaggggcaccgtggactccctgaacgagctgccgatcgacggcgagaactccctggtgggctactcccacaggctgtcccacgtgaccctgaccaggtccctgtacaacaccaacatcacctccctgccgaccttcgtgtggacccaccactccgccaccaacaccaacaccatcaacccggacatcatcacccagatcccgctggtgaagggcttcaggctgggcggcggcacctccgtgatcaagggcccgggcttcaccggcggcgacatcctgaggaggaacaccatcggcgagttcgtgtccctgcaggtgaacatcaactccccgatcacccagaggtacaggctgaggttcaggtacgcctcctccagggacgccaggatcaccgtggccatcggcggccagatcagggtggacatgaccctggagaagaccatggagatcggcgagtccctgacctccaggaccttctcctacaccaacttctccaacccgttctccttcagggccaacccggacatcatcaggatcgccgaggagctgccgatcaggggcggcgagctgtacatcgacaagatcgagctgatcctggccgacgccaccttcgaggaggagtacgacctggagagggcccagaaggccgtgaacgccctgttcacctccaccaaccagctgggcctgaagaccgacgtgaccgactaccacatcgaccaggtgtccaacctggtggagtgcctg

SEQ ID NO: 89 - DNA, 1992 bp, synthetic construct
  source 1..1992: mol_type = other DNA
atggaggagaacaaccagaaccagtgcatcccgtacaactgcctgtccaacccggaggaggtgctgctggacggcgagaggatctccaccggcaactcctccatcgacatctccctgtccctggtgcagttcctggtgtccaacttcgtgccgggcggcggcttcctggtgggcctgatcgacttcgtgtggggcatcgtgggcccgtcccagtgggacgccttcctggtgcagatcgagcagctgatcaacgagaggatcgccgagttcgccaggaacgccgccatcgccaacctggagggcctgggcaacaacttcaacatctacgtggaggccttcaaggagtgggaggaggacccgaacaacccggccaccaggaccagggtgatcgacaggttcaggatcctggacggcctgctggagagggacatcccgtccttcaggatctccggcttcgaggtgccgctgctgtccgtgtacgcccaggccgccaacctgcacctggccatcctgagggactccgtgatcttcggcgagaggtggggcctgaccaccatcaacgtgaacgagaactacaacaggctgatcaggcacatcgacgagtacgccgaccactgcgccaacacctacaacaggggcctgaacaacctgccgaagtccacctaccaggactggatcacctacaacaggctgaggagggacctgaccctgaccgtgctggacatcgccgccttcttcccgaactacgacaacaggaggtacccgatccagccggtgggcacctcctcccagctgaccagggaggtgtacaccgacccggtgatcaacatcaccgactacagggtgggcccgtccttcgagaacatcgagaactccgccatcaggtccccgcacctgatggacttcctgaacaacctgaccatcgacaccgacctgatcaggggcgtgcactactgggccggccacagggtgacctcccacttcaccggctcctcccaggtgatcaccaccccgcagtacggcatcaccgccaacgccgagccgaggaggaccatcgccccgtccaccttcccgggcctgaacctgttctacaggaccctgtccaacccgttcttcaggaggtccgagaacatcaccccgaccctgggcatcaacgtggtgcagggcgtgggcttcatccagccgaacaacgccgaggtgctgtacaggtccaggggcaccgtggactccctgaacgagctgccgatcgacggcgagaactccctggtgggctactcccacaggctgtcccacgtgaccctgaccaggtccctgtacaacaccaacatcacctccctgccgaccttcgtgtggacccaccactccgccaccctgaccaacaccatcgacccggagaggatcaaccagatcccgctggtgaagggcttcagggtgtggggcggcacctccgtgatcaccggcccgggcttcaccggcggcgacatcctgaggaggaacaccttcggcgacttcgtgtccctgcaggtgaacatcaactccccgatcacccagaggtacaggctgaggttcaggtacgcctcctccagggacgccagggtgatcgtgctgaccggcgccgcctccaccggcgtgggcggccaggtgtccgtgaacatgccgctgcagaagaccatggagatcggcgagaacctgacctccaggaccttcaggtacaccgacttctccaacccgttctccttcagggccaacccggacatcatcggcatctccgagcagccgctgttcggcgccggctccatctcctccggcgagctgtacatcgacaagatcgagatcatcctggccgacgccaccttcgaggccgagtccgacctggagagggcccagaaggccgtgaacgccctgttcacctcctccaaccagatcggcctgaagaccgacgtgaccgactaccacatcgaccaggtgtccaacctggtggactgcctgtcc

SEQ ID NO: 90 - DNA, 1953 bp, synthetic construct
  source 1..1953: mol_type = other DNA
atggaggagaacaaccagaaccagtgcatcccgtacaactgcctgtccaacccggaggaggtgctgctggacggcgagaggatctccaccggcaactcctccatcgacatctccctgtccctggtgcagttcctggtgtccaacttcgtgccgggcggcggcttcctggtgggcctgatcgacttcgtgtggggcatcgtgggcccgtcccagtgggacgccttcctggtgcagatcgagcagctgatcaacgagaggatcgccgagttcgccaggaacgccgccatcgccaacctggagggcctgggcaacaacttcaacatctacgtggaggccttcaaggagtgggaggaggacccgaacaacccggccaccaggaccagggtgatcgacaggttcaggatcctggacggcctgctggagagggacatcccgtccttcaggatctccggcttcgaggtgccgctgctgtccgtgtacgcccaggccgccaacctgcacctggccatcctgagggactccgtgatcttcggcgagaggtggggcctgaccaccatcaacgtgaacgagaactacaacaggctgatcaggcacatcgacgagtacgccgaccactgcgccaacacctacaacaggggcctgaacaacctgccgaagtccacctaccaggactggatcacctacaacaggctgaggagggacctgaccctgaccgtgctggacatcgccgccttcttcccgaactacgacaacaggaggtacccgatccagccggtgggccagctgaccagggaggtgtacaccgacccgctgatcaacttcaacccgcagctgcagtccgtggcccagctgccgaccttcaacgtgatggagtcctccgccatcaggaacccgcacctgttcgacatcctgaacaacctgaccatcttcaccgactggttctccgtgggcaggaacttctactggggcggccacagggtgatctcctccctgatcggcggcggcaacatcacctccccgatctacggcagggaggccaaccaggagccgccgaggtccttcaccttcaacggcccggtgttcaggaccctgtccaacccgaccctgaggctgctgcagcagccgtggccggccccgccgttcaacctgaggggcgtggagggcgtggagttctccaccccgaccaactccttcacctacaggggcaggggcaccgtggactccctgaccgagctgccgccggaggacaactccgtgccgccgagggagggctactcccacaggctgtgccacgccaccttcgtgcagaggtccggcaccccgttcctgaccaccggcgtggtgttctcctggacccacaggtccgccaccaacaccaacaccatcaacccggacatcatcacccagatcccgctggtgaagggcttcaggctgggcggcggcacctccgtgatcaagggcccgggcttcaccggcggcgacatcctgaggaggaacaccatcggcgagttcgtgtccctgcaggtgaacatcaactccccgatcacccagaggtacaggctgaggttcaggtacgcctcctccagggacgccaggatcaccgtggccatcggcggccagatcagggtggacatgaccctggagaagaccatggagatcggcgagtccctgacctccaggaccttctcctacaccaacttctccaacccgttctccttcagggccaacccggacatcatcaggatcgccgaggagctgccgatcaggggcggcgagctgtacatcgacaagatcgagctgatcctggccgacgccaccttcgaggaggagtacgacctggagagggcccagaaggccgtgaacgccctgttcacctccaccaaccagctgggcctgaagaccgacgtgaccgactaccacatcgaccaggtgtccaacctggtggagtgcctg

SEQ ID NO: 91 - DNA, 2103 bp, synthetic construct
  source 1..2103: mol_type = other DNA
atgaactccgtgctgaactccggcaggaccaccatctgcgacgcctacaacgtggtggcccacgacccgttctccttcgagcacaagtccctggacaccatccagaaggagtggatggagtggaagaggaccgaccactccctgtacgtggccccggtggtgggcaccgtgtcctccttcctgctgaagaaggtgggctccctgatcggcaagaggatcctgtccgagctgtggggcatcatcttcccgtccggctccaccaacctgatgcaggacatcctgagggagaccgagcagttcctgaaccagaggctgaacaccgacaccctggccagggtgaacgccgagctgatcggcctgcaggccaacatcagggagttcaaccagcaggtggacaacttcctgaacccgacccagaacccggtgccgctgtccatcacctcctccgtgaacaccatgcagcagctgttcctgaacaggctgccgcagttccagatccagggctaccagctgctgctgctgccgctgttcgcccaggccgccaacatgcacctgtccttcatcagggacgtgatcctgaacgccgacgagtggggcatctccgccgccaccctgaggacctacagggactacctgaggaactacaccagggactactccaactactgcatcaacacctaccagaccgccttcaggggcctgaacaccaggctgcacgacatgctggagttcaggacctacatgttcctgaacgtgttcgagtacgtgtccatctggtccctgttcaagtaccagtccctgatggtgtcctccggcgccaacctgtacgcctccggctccggcccgcagcagacccagtccttcaccgcccagaactggccgttcctgtactccctgttccaggtgaactccaactacatcctgtccggcatctccggcaccaggctgtccatcaccttcccgaacatcggcggcctgccgggctccaccaccacccactccctgaactccgccagggtgaactactccggcggcgtgtcctccggcctgatcggcgccaccaacctgaaccacaacttcaactgctccaccgtgctgccgccgctgtccaccccgttcgtgaggtcctggctggactccggcaccgacagggagggcgtggccacctccaccaactggcagaccgagtccttccagaccaccctgtccctgaggtgcggcgccttctccgccaggggcaactccaactacttcccggactacttcatcaggaacatctccggcgtgccgctggtgatcaggaacgaggacctgaccaggccgctgcactacaaccagatcaggaacatcgagtccccgtccggcaccccgggcggcgccagggcctacctggtgtccgtgcacaacaggaagaacaacatctacgccgccaacgagaacggcaccatgatccacctggccccggaggactacaccggcaccctgaccaacaccatcgacccggagaggatcaaccagatcccgctggtgaagggcttcagggtgtggggcggcacctccgtgatcaccggcccgggcttcaccggcggcgacatcctgaggaggaacaccttcggcgacttcgtgtccctgcaggtgaacatcaactccccgatcacccagaggtacaggctgaggttcaggtacgcctcctccagggacgccagggtgatcgtgctgaccggcgccgcctccaccggcgtgggcggccaggtgtccgtgaacatgccgctgcagaagaccatggagatcggcgagaacctgacctccaggaccttcaggtacaccgacttctccaacccgttctccttcagggccaacccggacatcatcggcatctccgagcagccgctgttcggcgccggctccatctcctccggcgagctgtacatcgacaagatcgagatcatcctggccgacgccaccttcgaggccgagtccgacctggagagggcccagaaggccgtgaacgccctgttcacctcctccaaccagatcggcctgaagaccgacgtgaccgactaccacatcgaccaggtgtccaacctggtggactgcctgtcc

SEQ ID NO: 92 - DNA, 1869 bp, synthetic construct
  source 1..1869: mol_type = other DNA
atgaactccgtgctgaactccggcaggaccaccatctgcgacgcctacaacgtggtggcccacgacccgttctccttcgagcacaagtccctggacaccatccagaaggagtggatggagtggaagaggaccgaccactccctgtacgtggccccggtggtgggcaccgtgtcctccttcctgctgaagaaggtgggctccctgatcggcaagaggatcctgtccgagctgtggggcatcatcttcccgtccggctccaccaacctgatgcaggacatcctgagggagaccgagcagttcctgaaccagaggctgaacaccgacaccctggccagggtgaacgccgagctgatcggcctgcaggccaacatcagggagttcaaccagcaggtggacaacttcctgaacccgacccagaacccggtgccgctgtccatcacctcctccgtgaacaccatgcagcagctgttcctgaacaggctgccgcagttccagatccagggctaccagctgctgctgctgccgctgttcgcccaggccgccaacatgcacctgtccttcatcagggacgtgatcctgaacgccgacgagtggggcatctccgccgccaccctgaggacctacagggactacctgaggaactacaccagggactactccaactactgcatcaacacctaccagaccgccttcaggggcctgaacaccaggctgcacgacatgctggagttcaggacctacatgttcctgaacgtgttcgagtacgtgtccatctggtccctgttcaagtaccagtcccagctgaccagggaggtgtacaccgacccgctgatcaacttcaacccgcagctgcagtccgtggcccagctgccgaccttcaacgtgatggagtcctccgccatcaggaacccgcacctgttcgacatcctgaacaacctgaccatcttcaccgactggttctccgtgggcaggaacttctactggggcggccacagggtgatctcctccctgatcggcggcggcaacatcacctccccgatctacggcagggaggccaaccaggagccgccgaggtccttcaccttcaacggcccggtgttcaggaccctgtccaacccgaccctgaggctgctgcagcagccgtggccggccccgccgttcaacctgaggggcgtggagggcgtggagttctccaccccgaccaactccttcacctacaggggcaggggcaccgtggactccctgaccgagctgccgccggaggacaactccgtgccgccgagggagggctactcccacaggctgtgccacgccaccttcgtgcagaggtccggcaccccgttcctgaccaccggcgtggtgttctcctggacccacaggtccgccaacatctacgccgccaacgagaacggcaccatgatccacctggccccggaggactacaccggcttcaccatctccccgatccacgccacccaggtgaacaaccagaccaggaccttcatctccgagaagttcggcaaccagggcgactccctgaggttcgagcagtccaacaccaccgccaggtacaccctgaggggcaacggcaactcctacaacctgtacctgagggtgtcctccatcggcaactccaccatcagggtgaccatcaacggcagggtgtacaccgtgtccaacgtgaacaccaccaccaacaacgacggcgtgaacgacaacggcgccaggttctccgacatcaacatcggcaacatcgtggcctccgacaacaccaacgtgaccctggacatcaacgtgaccctgaactccggcaccccgttcgacctgatgaacatcatgttcgtgccgaccaacctgccgccgctgtac

SEQ ID NO: 93 - DNA, 2010 bp, synthetic construct
  source 1..2010: mol_type = other DNA
atgaactccgtgctgaactccggcaggaccaccatctgcgacgcctacaacgtggtggcccacgacccgttctccttcgagcacaagtccctggacaccatccagaaggagtggatggagtggaagaggaccgaccactccctgtacgtggccccggtggtgggcaccgtgtcctccttcctgctgaagaaggtgggctccctgatcggcaagaggatcctgtccgagctgtggggcatcatcttcccgtccggctccaccaacctgatgcaggacatcctgagggagaccgagcagttcctgaaccagaggctgaacaccgacaccctggccagggtgaacgccgagctgatcggcctgcaggccaacatcagggagttcaaccagcaggtggacaacttcctgaacccgacccagaacccggtgccgctgtccatcacctcctccgtgaacaccatgcagcagctgttcctgaacaggctgccgcagttccagatccagggctaccagctgctgctgctgccgctgttcgcccaggccgccaacatgcacctgtccttcatcagggacgtgatcctgaacgccgacgagtggggcatctccgccgccaccctgaggacctacagggactacctgaggaactacaccagggactactccaactactgcatcaacacctaccagaccgccttcaggggcctgaacaccaggctgcacgacatgctggagttcaggacctacatgttcctgaacgtgttcgagtacgtgtccatctggtccctgttcaagtaccagtcccagctgaccagggaggtgtacaccgacccgctgatcaacttcaacccgcagctgcagtccgtggcccagctgccgaccttcaacgtgatggagtcctccgccatcaggaacccgcacctgttcgacatcctgaacaacctgaccatcttcaccgactggttctccgtgggcaggaacttctactggggcggccacagggtgatctcctccctgatcggcggcggcaacatcacctccccgatctacggcagggaggccaaccaggagccgccgaggtccttcaccttcaacggcccggtgttcaggaccctgtccaacccgaccctgaggctgctgcagcagccgtggccggccccgccgttcaacctgaggggcgtggagggcgtggagttctccaccccgaccaactccttcacctacaggggcaggggcaccgtggactccctgaccgagctgccgccggaggacaactccgtgccgccgagggagggctactcccacaggctgtgccacgccaccttcgtgcagaggtccggcaccccgttcctgaccaccggcgtggtgttctcctggacccacaggtccgccaccctgaccaacaccatcgacccggagaggatcaaccagatcccgctggtgaagggcttcagggtgtggggcggcacctccgtgatcaccggcccgggcttcaccggcggcgacatcctgaggaggaacaccttcggcgacttcgtgtccctgcaggtgaacatcaactccccgatcacccagaggtacaggctgaggttcaggtacgcctcctccagggacgccagggtgatcgtgctgaccggcgccgcctccaccggcgtgggcggccaggtgtccgtgaacatgccgctgcagaagaccatggagatcggcgagaacctgacctccaggaccttcaggtacaccgacttctccaacccgttctccttcagggccaacccggacatcatcggcatctccgagcagccgctgttcggcgccggctccatctcctccggcgagctgtacatcgacaagatcgagatcatcctggccgacgccaccttcgaggccgagtccgacctggagagggcccagaaggccgtgaacgccctgttcacctcctccaaccagatcggcctgaagaccgacgtgaccgactaccacatcgaccaggtgtccaacctggtggactgcctgtcc

SEQ ID NO: 94 - DNA, 1881 bp, synthetic construct
  source 1..1881: mol_type = other DNA
atggaggagaacaaccagaaccagtgcatcccgtacaactgcctgtccaacccggaggaggtgctgctggacggcgagaggatctccaccggcaactcctccatcgacatctccctgtccctggtgcagttcctggtgtccaacttcgtgccgggcggcggcttcctggtgggcctgatcgacttcgtgtggggcatcgtgggcccgtcccagtgggacgccttcctggtgcagatcgagcagctgatcaacgagaggatcgccgagttcgccaggaacgccgccatcgccaacctggagggcctgggcaacaacttcaacatctacgtggaggccttcaaggagtgggaggaggacccgaacaacccggccaccaggaccagggtgatcgacaggttcaggatcctggacggcctgctggagagggacatcccgtccttcaggatctccggcttcgaggtgccgctgctgtccgtgtacgcccaggccgccaacctgcacctggccatcctgagggactccgtgatcttcggcgagaggtggggcctgaccaccatcaacgtgaacgagaactacaacaggctgatcaggcacatcgacgagtacgccgaccactgcgccaacacctacaacaggggcctgaacaacctgccgaagtccacctaccaggactggatcacctacaacaggctgaggagggacctgaccctgaccgtgctggacatcgccgccttcttcccgaactacgacaacaggaggtacccgatccagccggtgggcctgatggtgtcctccggcgccaacctgtacgcctccggctccggcccgcagcagacccagtccttcaccgcccagaactggccgttcctgtactccctgttccaggtgaactccaactacatcctgtccggcatctccggcaccaggctgtccatcaccttcccgaacatcggcggcctgccgggctccaccaccacccactccctgaactccgccagggtgaactactccggcggcgtgtcctccggcctgatcggcgccaccaacctgaaccacaacttcaactgctccaccgtgctgccgccgctgtccaccccgttcgtgaggtcctggctggactccggcaccgacagggagggcgtggccacctccaccaactggcagaccgagtccttccagaccaccctgtccctgaggtgcggcgccttctccgccaggggcaactccaactacttcccggactacttcatcaggaacatctccggcgtgccgctggtgatcaggaacgaggacctgaccaggccgctgcactacaaccagatcaggaacatcgagtccccgtccggcaccccgggcggcgccagggcctacctggtgtccgtgcacaacaggaagaacaacatctacgccgccaacgagaacggcaccatgatccacctggccccggaggactacaccggcttcaccatctccccgatccacgccacccaggtgaacaaccagaccaggaccttcatctccgagaagttcggcaaccagggcgactccctgaggttcgagcagtccaacaccaccgccaggtacaccctgaggggcaacggcaactcctacaacctgtacctgagggtgtcctccatcggcaactccaccatcagggtgaccatcaacggcagggtgtacaccgtgtccaacgtgaacaccaccaccaacaacgacggcgtgaacgacaacggcgccaggttctccgacatcaacatcggcaacatcgtggcctccgacaacaccaacgtgaccctggacatcaacgtgaccctgaactccggcaccccgttcgacctgatgaacatcatgttcgtgccgaccaacctgccgccgctgtac

SEQ ID NO: 95 - DNA, 2085 bp, synthetic construct
  source 1..2085: mol_type = other DNA
atggaggagaacaaccagaaccagtgcatcccgtacaactgcctgtccaacccggaggaggtgctgctggacggcgagaggatctccaccggcaactcctccatcgacatctccctgtccctggtgcagttcctggtgtccaacttcgtgccgggcggcggcttcctggtgggcctgatcgacttcgtgtggggcatcgtgggcccgtcccagtgggacgccttcctggtgcagatcgagcagctgatcaacgagaggatcgccgagttcgccaggaacgccgccatcgccaacctggagggcctgggcaacaacttcaacatctacgtggaggccttcaaggagtgggaggaggacccgaacaacccggccaccaggaccagggtgatcgacaggttcaggatcctggacggcctgctggagagggacatcccgtccttcaggatctccggcttcgaggtgccgctgctgtccgtgtacgcccaggccgccaacctgcacctggccatcctgagggactccgtgatcttcggcgagaggtggggcctgaccaccatcaacgtgaacgagaactacaacaggctgatcaggcacatcgacgagtacgccgaccactgcgccaacacctacaacaggggcctgaacaacctgccgaagtccacctaccaggactggatcacctacaacaggctgaggagggacctgaccctgaccgtgctggacatcgccgccttcttcccgaactacgacaacaggaggtacccgatccagccggtgggcctgatggtgtcctccggcgccaacctgtacgcctccggctccggcccgcagcagacccagtccttcaccgcccagaactggccgttcctgtactccctgttccaggtgaactccaactacatcctgtccggcatctccggcaccaggctgtccatcaccttcccgaacatcggcggcctgccgggctccaccaccacccactccctgaactccgccagggtgaactactccggcggcgtgtcctccggcctgatcggcgccaccaacctgaaccacaacttcaactgctccaccgtgctgccgccgctgtccaccccgttcgtgaggtcctggctggactccggcaccgacagggagggcgtggccacctccaccaactggcagaccgagtccttccagaccaccctgtccctgaggtgcggcgccttctccgccaggggcaactccaactacttcccggactacttcatcaggaacatctccggcgtgccgctggtgatcaggaacgaggacctgaccaggccgctgcactacaaccagatcaggaacatcgagtccccgtccggcaccccgggcggcgccagggcctacctggtgtccgtgcacaacaggaagaacaacatctacgccgccaacgagaacggcaccatgatccacctggccccggaggactacaccggcaccctgaccaacaccatcgacccggagaggatcaaccagatcccgctggtgaagggcttcagggtgtggggcggcacctccgtgatcaccggcccgggcttcaccggcggcgacatcctgaggaggaacaccttcggcgacttcgtgtccctgcaggtgaacatcaactccccgatcacccagaggtacaggctgaggttcaggtacgcctcctccagggacgccagggtgatcgtgctgaccggcgccgcctccaccggcgtgggcggccaggtgtccgtgaacatgccgctgcagaagaccatggagatcggcgagaacctgacctccaggaccttcaggtacaccgacttctccaacccgttctccttcagggccaacccggacatcatcggcatctccgagcagccgctgttcggcgccggctccatctcctccggcgagctgtacatcgacaagatcgagatcatcctggccgacgccaccttcgaggccgagtccgacctggagagggcccagaaggccgtgaacgccctgttcacctcctccaaccagatcggcctgaagaccgacgtgaccgactaccacatcgaccaggtgtccaacctggtggactgcctgtcc

SEQ ID NO: 96 - DNA, 1788 bp, synthetic construct
  source 1..1788: mol_type = other DNA
atggaggagaacaaccagaaccagtgcatcccgtacaactgcctgtccaacccggaggaggtgctgctggacggcgagaggatctccaccggcaactcctccatcgacatctccctgtccctggtgcagttcctggtgtccaacttcgtgccgggcggcggcttcctggtgggcctgatcgacttcgtgtggggcatcgtgggcccgtcccagtgggacgccttcctggtgcagatcgagcagctgatcaacgagaggatcgccgagttcgccaggaacgccgccatcgccaacctggagggcctgggcaacaacttcaacatctacgtggaggccttcaaggagtgggaggaggacccgaacaacccggccaccaggaccagggtgatcgacaggttcaggatcctggacggcctgctggagagggacatcccgtccttcaggatctccggcttcgaggtgccgctgctgtccgtgtacgcccaggccgccaacctgcacctggccatcctgagggactccgtgatcttcggcgagaggtggggcctgaccaccatcaacgtgaacgagaactacaacaggctgatcaggcacatcgacgagtacgccgaccactgcgccaacacctacaacaggggcctgaacaacctgccgaagtccacctaccaggactggatcacctacaacaggctgaggagggacctgaccctgaccgtgctggacatcgccgccttcttcccgaactacgacaacaggaggtacccgatccagccggtgggccagctgaccagggaggtgtacaccgacccgctgatcaacttcaacccgcagctgcagtccgtggcccagctgccgaccttcaacgtgatggagtcctccgccatcaggaacccgcacctgttcgacatcctgaacaacctgaccatcttcaccgactggttctccgtgggcaggaacttctactggggcggccacagggtgatctcctccctgatcggcggcggcaacatcacctccccgatctacggcagggaggccaaccaggagccgccgaggtccttcaccttcaacggcccggtgttcaggaccctgtccaacccgaccctgaggctgctgcagcagccgtggccggccccgccgttcaacctgaggggcgtggagggcgtggagttctccaccccgaccaactccttcacctacaggggcaggggcaccgtggactccctgaccgagctgccgccggaggacaactccgtgccgccgagggagggctactcccacaggctgtgccacgccaccttcgtgcagaggtccggcaccccgttcctgaccaccggcgtggtgttctcctggacccacaggtccgccttcaccatctccccgatccacgccacccaggtgaacaaccagaccaggaccttcatctccgagaagttcggcaaccagggcgactccctgaggttcgagcagtccaacaccaccgccaggtacaccctgaggggcaacggcaactcctacaacctgtacctgagggtgtcctccatcggcaactccaccatcagggtgaccatcaacggcagggtgtacaccgtgtccaacgtgaacaccaccaccaacaacgacggcgtgaacgacaacggcgccaggttctccgacatcaacatcggcaacatcgtggcctccgacaacaccaacgtgaccctggacatcaacgtgaccctgaactccggcaccccgttcgacctgatgaacatcatgttcgtgccgaccaacctgccgccgctgtac

SEQ ID NO: 97 - protein, 664 aa, Bacillus thuringiensis
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 98 - protein, 260 aa, Bacillus thuringiensis
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVG

SEQ ID NO: 99 - protein, 196 aa, Bacillus thuringiensis
QLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSA

SEQ ID NO: 100 - protein, 208 aa, Bacillus thuringiensis
TLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLS

SEQ ID NO: 101 - protein, 690 aa, Bacillus thuringiensis
MTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNQEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTSAQLTREIYTDPIGRTNAPSGFASTNWFNNNAPSFSAIEAAIIRPPHLLDFPEQLTIYSASSRWSSTQHMIYWVGHRLNFRPIGGTLNTSTQGLTNNTSINPVTLQFTSRDVYRTESYAGTNILFTTPVNGVPWARFNFINPQNIYERGTTTYSQPYQGVGIQLFDSETELPPETTERPNYESYSHRLSHIGLIIGNTLRAPVYSWTHRSADRTNTIGPNRITQIPLVKALNLHSGVTGVRGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSD

SEQ ID NO: 102 - protein, 282 aa, Bacillus thuringiensis
MTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNQEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPIN

SEQ ID NO: 103 - protein, 213 aa, Bacillus thuringiensis
TSAQLTREIYTDPIGRTNAPSGFASTNWFNNNAPSFSAIEAAIIRPPHLLDFPEQLTIYSASSRWSSTQHMIYWVGHRLNFRPIGGTLNTSTQGLTNNTSINPVTLQFTSRDVYRTESYAGTNILFTTPVNGVPWARFNFINPQNIYERGTTTYSQPYQGVGIQLFDSETELPPETTERPNYESYSHRLSHIGLIIGNTLRAPVYSWTHRSAD

SEQ ID NO: 104 - protein, 195 aa, Bacillus thuringiensis
RTNTIGPNRITQIPLVKALNLHSGVTGVRGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSD

SEQ ID NO: 105 - protein, 657 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSD

SEQ ID NO: 106 - protein, 258 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIR

SEQ ID NO: 107 - protein, 204 aa, Bacillus thuringiensis
TVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEF

SEQ ID NO: 108 - protein, 195 aa, Bacillus thuringiensis
NNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSD

SEQ ID NO: 109 - protein, 157 aa, Bacillus thuringiensis
NNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLER

SEQ ID NO: 110 - protein, 648 aa, Bacillus thuringiensis
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIPTSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSATNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATFEEEYDLERAQKAVNALFTSTNQLGLKTDVTDYHIDQVSNLVECL

SEQ ID NO: 111 - protein, 257 aa, Bacillus thuringiensis
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIP

SEQ ID NO: 112 - protein, 196 aa, Bacillus thuringiensis
TSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSA

SEQ ID NO: 113 - protein, 195 aa, Bacillus thuringiensis
TNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATFEEEYDLERAQKAVNALFTSTNQLGLKTDVTDYHIDQVSNLVECL

SEQ ID NO: 114 - protein, 640 aa, Bacillus thuringiensis
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYARAFSDWEEDPTNPALREEMRIQFNDMNSALITAIPLFRVQNYEVALLSVYVQAANLHLSILRDISVFGERWGYDTATINNRYSDLTSLIHVYTNHCVDTYNQGLKLLEGRFLSDWIVYNRFRRQLTISVLDIVAFFPNYDIRAYPIQTATQLSREVYLDLPFINENLSPAASYPTFSAAESAIIRSPHLVDFLNNFTIYTDSLASYAYWGGHRVRSFRTGTTTNLIQSPLYGREGNTERPVTIWASSSVPIFRTLSYVTGLNNRNPVAGIEGVEFQDTTGRSIYRKSGPIDSFSELPPQDANISPTIGYSHRLCHATFLERISGPRIAGVVFSWTHRSASPTNEVSPSRITQIPWVKANTLASGASVIKGPGFTGGDILTRNSMGDLGSLRVTFTGRLPQSYYIRFRYASVANRSGSFRYSQPPSYGISFPKTMDAGEPLTSRSFALTTLFTPITFSRAQEEFDLYIQTGVYIDRIEFIPVTATLEAEYDLERAQEAVNALFTSSNQLGLKTDVTDYHIDQVSNLVDCLS

SEQ ID NO: 115 - protein, 254 aa, Bacillus thuringiensis
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYARAFSDWEEDPTNPALREEMRIQFNDMNSALITAIPLFRVQNYEVALLSVYVQAANLHLSILRDISVFGERWGYDTATINNRYSDLTSLIHVYTNHCVDTYNQGLKLLEGRFLSDWIVYNRFRRQLTISVLDIVAFFPNYDIRAY

SEQ ID NO: 116 - protein, 205 aa, Bacillus thuringiensis
PIQTATQLSREVYLDLPFINENLSPAASYPTFSAAESAIIRSPHLVDFLNNFTIYTDSLASYAYWGGHRVRSFRTGTTTNLIQSPLYGREGNTERPVTIWASSSVPIFRTLSYVTGLNNRNPVAGIEGVEFQDTTGRSIYRKSGPIDSFSELPPQDANISPTIGYSHRLCHATFLERISGPRIAGVVFSWTHRSASPTNEVSPSR

SEQ ID NO: 117 - protein, 181 aa, Bacillus thuringiensis
ITQIPWVKANTLASGASVIKGPGFTGGDILTRNSMGDLGSLRVTFTGRLPQSYYIRFRYASVANRSGSFRYSQPPSYGISFPKTMDAGEPLTSRSFALTTLFTPITFSRAQEEFDLYIQTGVYIDRIEFIPVTATLEAEYDLERAQEAVNALFTSSNQLGLKTDVTDYHIDQVSNLVDCLS

SEQ ID NO: 118 - protein, 648 aa, Bacillus thuringiensis
MKNNIQNQCVPYNCLSNPEVEILSEERSTGRLPLDISLSLTRFLLSEFVPGVGVAFGLFDLIWGFITPSEWSLFLLQIEQLIEQRIETLERNRAITTLRGLADSYEVYLEALREWEENPNNAQLREDVRIRFANTDDALITAINNFTLTSFEIPLLSVYVQAANLHLSLLRDAVSFGQGWGLDIATVNNHYNRLINLIHRYTEHCLDTYNQGLENLRGTNTRQWSRFNQFRRELTLTVLDIVALFPNYDARAYPIQTSSQLTREIYTSSVIEDSPVSANIPNGFNRAEFGVRPPHLMDFMNSLFVTAETVRSQTVWGGHLVSSRNTAGNPINFPIYGIFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEIAGSDSWRAPMFSWTHRSADRTNIINPNIITQIPAVKAHNLHSGSTVVRGPGFTGGDLLRRTNTGTFADIRVNITGPLSQRYRVRIRYASTTDLQFFTRINGTSVNQGNFQRTMNRGGNLESGNFRTAGFSTPFSFSNAQSTFTLGTQAFSNQEVYIDRIEFVPAEVTFEAESDLERAQKAVNALFTSTSQLGLKTNVTGYHIDQVSNLVACLS

SEQ ID NO: 119 - protein, 253 aa, Bacillus thuringiensis
MKNNIQNQCVPYNCLSNPEVEILSEERSTGRLPLDISLSLTRFLLSEFVPGVGVAFGLFDLIWGFITPSEWSLFLLQIEQLIEQRIETLERNRAITTLRGLADSYEVYLEALREWEENPNNAQLREDVRIRFANTDDALITAINNFTLTSFEIPLLSVYVQAANLHLSLLRDAVSFGQGWGLDIATVNNHYNRLINLIHRYTEHCLDTYNQGLENLRGTNTRQWSRFNQFRRELTLTVLDIVALFPNYDARAY

SEQ ID NO: 120 - protein, 202 aa, Bacillus thuringiensis
PIQTSSQLTREIYTSSVIEDSPVSANIPNGFNRAEFGVRPPHLMDFMNSLFVTAETVRSQTVWGGHLVSSRNTAGNPINFPIYGIFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEIAGSDSWRAPMFSWTHRSADR

SEQ ID NO: 121 - protein, 193 aa, Bacillus thuringiensis
TNIINPNIITQIPAVKAHNLHSGSTVVRGPGFTGGDLLRRTNTGTFADIRVNITGPLSQRYRVRIRYASTTDLQFFTRINGTSVNQGNFQRTMNRGGNLESGNFRTAGFSTPFSFSNAQSTFTLGTQAFSNQEVYIDRIEFVPAEVTFEAESDLERAQKAVNALFTSTSQLGLKTNVTGYHIDQVSNLVACLS

SEQ ID NO: 122 - protein, 633 aa, Bacillus thuringiensis
MNSVLNSGRTTICDAYNVVAHDPFSFEHKSLDTIQKEWMEWKRTDHSLYVAPVVGTVSSFLLKKVGSLIGKRILSELWGIIFPSGSTNLMQDILRETEQFLNQRLNTDTLARVNAELIGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSLMVSSGANLYASGSGPQQTQSFTAQNWPFLYSLFQVNSNYILSGISGTRLSITFPNIGGLPGSTTTHSLNSARVNYSGGVSSGLIGATNLNHNFNCSTVLPPLSTPFVRSWLDSGTDREGVATSTNWQTESFQTTLSLRCGAFSARGNSNYFPDYFIRNISGVPLVIRNEDLTRPLHYNQIRNIESPSGTPGGARAYLVSVHNRKNNIYAANENGTMIHLAPEDYTGFTISPIHATQVNNQTRTFISEKFGNQGDSLRFEQSNTTARYTLRGNGNSYNLYLRVSSIGNSTIRVTINGRVYTVSNVNTTTNNDGVNDNGARFSDINIGNIVASDNTNVTLDINVTLNSGTPFDLMNIMFVPTNLPPLY

SEQ ID NO: 123 - protein, 266 aa, Bacillus thuringiensis
MNSVLNSGRTTICDAYNVVAHDPFSFEHKSLDTIQKEWMEWKRTDHSLYVAPVVGTVSSFLLKKVGSLIGKRILSELWGIIFPSGSTNLMQDILRETEQFLNQRLNTDTLARVNAELIGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQS

SEQ ID NO: 124 - protein, 227 aa, Bacillus thuringiensis
LMVSSGANLYASGSGPQQTQSFTAQNWPFLYSLFQVNSNYILSGISGTRLSITFPNIGGLPGSTTTHSLNSARVNYSGGVSSGLIGATNLNHNFNCSTVLPPLSTPFVRSWLDSGTDREGVATSTNWQTESFQTTLSLRCGAFSARGNSNYFPDYFIRNISGVPLVIRNEDLTRPLHYNQIRNIESPSGTPGGARAYLVSVHNRKNNIYAANENGTMIHLAPEDYTG

SEQ ID NO: 125 - protein, 140 aa, Bacillus thuringiensis
FTISPIHATQVNNQTRTFISEKFGNQGDSLRFEQSNTTARYTLRGNGNSYNLYLRVSSIGNSTIRVTINGRVYTVSNVNTTTNNDGVNDNGARFSDINIGNIVASDNTNVTLDINVTLNSGTPFDLMNIMFVPTNLPPLY